(19) United States
(12) Patent Application Publication
(10) Pub. No.: US 2016/0340695 A1
(43) Pub. Date: Nov. 24, 2016

(54) METHOD OF PRODUCING ISOPRENE MONOMER

(71) Applicant: Ajinomoto Co., Inc., Tokyo (JP)

(72) Inventors: Yoshinori TAJIMA, Kawasaki-shi (JP); Akiko ONUKI, Kawasaki-shi (JP); Hiroaki RACHI, Kawasaki-shi (JP)

(73) Assignee: Ajinomoto Co., Inc., Tokyo (JP)

(21) Appl. No.: 15/165,362

(22) Filed: May 26, 2016

Related U.S. Application Data
(63) Continuation of application No. PCT/JP2014/081645, filed on Nov. 28, 2014.

(30) Foreign Application Priority Data
Nov. 28, 2013 (JP) 2013-246350

(51) Int. Cl. C12P 5/02; C08K 13/02; C08F 136/08; C12N 9/88; C12N 9/16

(52) U.S. Cl. CPC C12P 5/026; C12N 9/88; C12Y 402/03027; C12N 9/16; C12Y 301/03081; C08F 136/08; C08K 13/02

(57) ABSTRACT

Isoprene synthase-expressing microorganisms that exhibit improved expression of pyrophosphate phosphatase are useful for producing isoprene monomer.

### CROSS REFERENCES TO RELATED APPLICATIONS

This application is a continuation of International Patent Application No. PCT/JP2014/081645, filed on Nov. 28, 2014, and claims priority to Japanese Patent Application No. 2013-246350, filed on Nov. 28, 2013, both of which are incorporated herein by reference in their entireties.

### BACKGROUND OF THE INVENTION

Field of the Invention

The present invention relates to isoprene synthase-expressing microorganisms that exhibit improved expression of pyrophosphate phosphatase, and methods of producing an isoprene monomer using such an isoprene synthase-expressing microorganism.

Discussion of the Background

Natural rubbers are very important raw materials in the tire and rubber industries. While demands for rubbers will expand in motorization mainly in developing countries in future, increase of farm plantations is not easy due to regulation to deforestation and competition with palms. Thus, it is predicted that the increase of natural rubber yields is difficult to be anticipated and a balance of demand and supply will become tight. Synthesized polyisoprene is available as a material in place of the natural rubber, and its raw material monomer (isoprene (2-methyl-1,3-butadiene)) is obtained by extracting from a C5 fraction obtained by cracking of naphtha. However in recent years, with lightening of a field of crackers, a production amount of isoprene has tended to decrease, and its supply has been apprehended. Also in recent years, due to strong influence of variation in oil prices, establishment of a system for inexpensively producing isoprene derived from non-oil resource has been required for stably securing an isoprene monomer.

For such a request, a method of producing the isoprene monomer using a transformant obtained by integrating an isolated isoprene synthase gene derived from kudzu or poplar and its mutant into a bacterium for fermentation production has been disclosed (see Japanese Laid-Open Publication No. 2011-505841, Japanese Laid-Open Publication No. 2011-518564, International Publication WO2010/031076, and International Publication WO2014/052054, all of which are incorporated herein by reference in their entireties).

The reaction mechanism of isoprene synthase has been already demonstrated, and the isoprene synthase acts upon DMAPP (dimethylallyl pyrophosphate) as a substrate to form pyrophosphate and isoprene (see Gary M. Silver and Ray Fall, Plant Physiol., (1991) 97:1588-1591, which is incorporated herein by reference in its entirety). As an effect of pyrophosphate that is a product on an activity of isoprene synthase, it has been known that an enzyme activity of isoprene synthase derived from willow (Salix discolor L.) is decreased by 1 mM sodium pyrophosphate (see Mary C. Wildermuth and Ray Fall, Plant Physiol., (1998) 116:1111-1123, which is incorporated herein by reference in its entirety). It has been known that an intracellular concentration of pyrophosphate is kept at about 0.5 mM by a protein having a pyrophosphate phosphatase activity in wild type strains of Escherichia coli and the like widely used as fermentative production bacteria (see Kukko-Kalske E., et al., J. Bacteriol., (1989) 171:4498-4500, which is incorporated herein by reference in its entirety).

However, there is no finding for the concentrations of pyrophosphate in microorganisms that produce excessive isoprene. Therefore, it has been unknown whether pyrophosphate reduces an activity of isoprene synthase in such a microorganism. Also, it has been unknown whether pyrophosphate has an effect on an ability to produce isoprene.

### SUMMARY OF THE INVENTION

Accordingly, it is one object of the present invention to provide novel biological methods that are excellent in the production of isoprene.

These and other objects, which will become apparent during the following detailed description, have been achieved by the inventors' discovery that the ability to produce isoprene monomer is improved by improving the expression amount of pyrophosphate phosphatase in an isoprene synthase-expressing microorganism.

Namely, the present invention provides:

(1) An isoprene synthase-expressing microorganism that exhibits improved expression of pyrophosphate phosphatase.

(2) The isoprene synthase-expressing microorganism according to (1), wherein said microorganism is a microorganism transformed with an expression vector for isoprene synthase.

(3) The isoprene synthase-expressing microorganism according to (1) or (2), wherein pyrophosphate phosphatase is homologous to said microorganism.

(4) The isoprene synthase-expressing microorganism according to (3), wherein expression of the pyrophosphate phosphatase is improved by modification of a promoter region of a pyrophosphate phosphatase gene inherent to said microorganism.

(5) The isoprene synthase-expressing microorganism according to any one of (1) to (4), wherein the expression of the pyrophosphate phosphatase is improved by increased copy number of the pyrophosphate phosphatase gene on a chromosome.

(6) The isoprene synthase-expressing microorganism according to any one of (1) to (5), wherein said microorganism is a microorganism belonging to Enterobacteriaceae.

(7) The isoprene synthase-expressing microorganism according to any one of (1) to (6), wherein said microorganism has an ability to synthesize dimethylallyl diphosphate via a methylerythritol diphosphate pathway.

(8) The isoprene synthase-expressing microorganism according to (7), wherein said microorganism is a bacterium belonging to genus Escherichia.

(9) The isoprene synthase-expressing microorganism according to (8), wherein said bacterium belonging to genus Escherichia is Escherichia coli.

(10) The isoprene synthase-expressing microorganism according to any one of (1) to (7), wherein said microorganism has an ability to synthesize dimethylallyl diphosphate via a mevalonate pathway.

(11) The isoprene synthase-expressing microorganism according to any one of (1) to (7) and (10), wherein said microorganism is a bacterium belonging to genus Pantoea.

(12) The isoprene synthase-expressing microorganism according to (11), wherein said bacterium belonging to genus Pantoea is Pantoea ananatis.

(13) A method of producing an isoprene monomer, comprising producing the isoprene monomer by culturing the isoprene synthase-expressing microorganism according to any one of (1) to (12) in culture medium.

(14) A method of producing an isoprene polymer, comprising

(I) producing an isoprene monomer by the method according to (13); and

(II) polymerizing the isoprene monomer to produce the isoprene polymer.

(15) A polymer derived from an isoprene monomer produced by the method according to (13).

(16) A rubber composition comprising the polymer according to (15).

(17) A tire produced by using the rubber composition according to (16).

The isoprene synthase-expressing microorganism of the present invention is excellent in ability to produce isoprene, and also remarkably improves the glucose yield.

In the isoprene synthase-expressing microorganism of the present invention, its growth is also ameliorated with improvement of the expression of pyrophosphate phosphatase (see FIG. 13).

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows an analysis of PPA expression by SDS-PAGE. Controls in lanes 1 and 2 denote samples prepared from MG1655 Ptac-KKDyI strain. Ptac-ppa in lanes 3 and 4 denotes samples prepared from MG1655 Ptac-KKDyI Ptac-ppa strain. M denotes protein molecular weight markers;

FIG. 2 shows amounts of isoprene generated per unit weight of dry leaves from various plants;

FIG. 3 shows amounts of isoprene generated per total protein mass extracted from leaves of various plants;

FIG. 4 shows an outline of mevalonate pathway downstream and its surrounding region in chromosome fixation;

FIG. 5 shows an outline of mevalonate pathway downstream and its surrounding region controlled by a tac promoter on a chromosome;

FIG. 6 shows a map of the plasmid pAH162-Para-mvaES;

FIG. 7 shows a map of the plasmid pAH162-KKDyI-ispS(K);

FIG. 8 shows a map of pAH162-Ptac-ispS(M)-mvk(Mma);

FIG. 9 shows construction of a modified chromosome ΔampC::KKDyI-ispS(K). (A) λRed dependent substitution of ampC gene with PCR formed DNA fragment attLphi80-kan-attRphi80. (B) phi80 Int dependent integration of plasmid pAH162-KKDyI-ispS(K). (C) phi80Int/Xis dependent removal of vector portion of pAH162-KKDyI-ispS(K);

FIG. 10 shows construction of a modified chromosome ΔampC::Para-mvaES. (A) λRed dependent substitution of ampH gene with PCR formed DNA fragment attLphi80-kan-attRphi80. (B) phi80 Int dependent integration of plasmid pAH162-Para-mvaES. (C) phi80 Int/Xis dependent removal of vector portion of pAH162-Para-mvaES;

FIG. 11 shows construction of the modified genome Δcrt::KKDyI-ispS(K) of megaplasmid pEA320. (A) Structure of P. ananatis crt locus arranged in megaplasmid pEA320. (B) λRed dependent substitution of crt operon with PCR formed DNA fragment attLphi80-kan-attRphi80. (C) phi80 Int dependent integration of plasmid pAH162-Ptac-ispS(M)-mvk(Mma). (D) phi80 Int/Xis dependent removal of vector portion of pAH162-Ptac-ispS(M)-mvk(Mma);

FIG. 12 shows amounts of protein expression of pyrophosphate phosphatase in AG10265 strain. 21.7 kD denotes an assumed molecular weight of pyrophosphate phosphatase encoded by a PAJ 2344(ppa-1) gene, and 19.8 kD denotes an assumed molecular weight of pyrophosphate phosphatase encoded by a PAJ_2736 (ppa-2) gene. Lane 1: soluble protein derived from AG10265 strain, Lanes 2 to 4: soluble protein derived from AG10265 Ptac-φ10-ppa1 strain, lanes 5 to 6: soluble protein derived from AG10265 Ptac-φ10-ppa2 strain, and M: molecular weight markers.

FIG. 13 shows growth change of P. ananatis isoprene-producing bacterium in jar cultivation;

FIG. 14 shows amounts of isoprene produced by P. ananatis isoprene-producing bacterium in jar cultivation;

The amount (mg) of isoprene produced per batch is shown;

FIG. 15 shows a map of pAH162-mvaES;

FIG. 16 shows a plasmid pAH162-MCS-mvaES for chromosome fixation;

FIG. 17 shows a set of plasmid for chromosome fixation holding a mvaES gene under transcription control by P_{phoC};

FIG. 18 shows construction of an integrative vector pAH162-λattL-KmR-λattR;

FIG. 19 shows an integrative expression vector pAH162-Ptac;

FIG. 20 shows optimization of codons in chemically synthesized operon KDyI;

FIG. 21 shows integrative plasmids (A) pAH162-Tc-Ptac-KDyI and (B) pAH162-Km-Ptac-KDyI holding operon KDyI having optimized codons;

FIG. 22 shows an integrative plasmid holding a mevalonate kinase gene derived from M. paludicola;

FIG. 23 shows maps of the modified genomes: (A) ΔampC::attBₚₕᵢ₈₀, (B) ΔampH::attBₚₕᵢ₈₀ and (C) Δcrt::attBₚₕᵢ₈₀;

FIG. 24 shows maps of the modified genomes: (A) Δcrt::pAH162-P_{tac}-mvk(X) and (B) Δcrt::P_{tac}-mvk(X);

FIG. 25 shows maps of the modified genomes: (A) ΔampH::pAH162-Km-P_{tac}-KDyI, and (B) ΔampC::pAH162-Km-P_{tac}-KDyI;

FIG. 26 shows maps of the modified genomes (A) ΔampH::pAH162-Para-mvaES and (B) ΔampC::pAH162-Para-mvaES; and

FIG. 27 shows confirmation of PPA expression in SWITCH-PphoC-1(S)ΔydcI::Ptac-MG-ppa. Cont and MG-ppa denote that a sample derived from SWITCH-PphoC-1(S)ΔydcI strain and a sample derived from SWITCH-PphoC-1(S)ΔydcI::Ptac-MG-ppa strain were electrophoresed, respectively.

### DETAILED DESCRIPTION OF THE PREFERRED EMBODIMENTS

The present invention provides isoprene-expressing microorganisms that exhibit improved expression of pyrophosphate phosphatase.

Pyrophosphate phosphatase is an enzyme that hydrolyzes a pyrophosphoric acid into a bimolecular phosphoric acid. Examples of the pyrophosphate phosphatase include pyrophosphate phosphatase derived from a microorganism as a host as described below. For the pyrophosphate phosphatase, pyrophosphate phosphatase derived from a microorganism belonging to the family Enterobacteriaceae, in particular, a microorganism belonging to the family Enterobacteriaceae among microorganisms as described below, is also preferred.

Specifically, the pyrophosphate phosphatase may be a protein consisting of the amino acid sequence of SEQ ID NO:136, SEQ ID NO:137, or SEQ ID NO:138.

Further, the pyrophosphate phosphatase may be a protein that comprises an amino acid sequence having 70% or more amino acid sequence identity to the amino acid sequence of SEQ ID NO:136, SEQ ID NO:137, or SEQ ID NO:138, and has a pyrophosphate phosphatase activity. The amino acid sequence percent identity may be, for example, 75% or more, 80% or more, 85% or more, 90% or more, 95% or more, 96% or more, 97% or more, 98% or more, or 99% or more. The pyrophosphate phosphatase activity refers to an activity of hydrolyzing a pyrophosphoric acid into a bimolecular phosphoric acid.

Further, the pyrophosphate phosphatase may be a protein that comprises an amino acid sequence having a mutation of one or several amino acid residues in the amino acid sequence of SEQ ID NO:136, SEQ ID NO:137, or SEQ ID NO:138, and has a pyrophosphate phosphatase activity. Examples of the mutation of the amino acid residues may include deletion, substitution, addition, and insertion of amino acid residues. The mutation of one or several amino acid residues may be introduced into one region or multiple different regions in the amino acid sequence. The term “one or several” indicates a range in which a three-dimensional structure and an activity of the protein are not impaired greatly. In the case of the protein, the number represented by “one or several” is, for example, 1 to 50, preferably 1 to 40, more preferably 1 to 30, 1 to 20, 1 to 10, or 1 to 5.

The pyrophosphate phosphatase preferably has a pyrophosphate phosphatase activity that is 50% or more, 60% or more, 70% or more, 80% or more, 90% or more, or 95% or more of the pyrophosphate phosphatase activity of the protein consisting of the amino acid sequence of SEQ ID NO:136, SEQ ID NO:137, or SEQ ID NO:138 when measured under the same conditions (e.g., buffer, concentration, temperature, and reaction time).

The identity of the amino acid sequences can be determined, for example, using the algorithm BLAST (Pro. Natl. Acad. Sci. USA, 90, 5873 (1993) which is incorporated herein by reference in its entirety) by Karlin and Altschul, and the FASTA algorithm (Methods Enzymol., 183, 63 (1990) which is incorporated herein by reference in its entirety) by Pearson. The program referred to as BLASTP was developed based on the algorithm BLAST (see http://www.ncbi.nlm.nih.gov, which is incorporated herein by reference in its entirety). Thus, the identity of the amino acid sequences may be calculated using this program with default setting. Also, for example, a numerical value obtained by calculating similarity as a percentage at a setting of “unit size to compare=2” using the full length of a polypeptide portion encoded in ORF with the software GENETYX Ver. 7.0.9 from Genetyx Corporation employing the Lipman-Pearson method may be used as the identity of the amino acid sequences. The lowest value among the values derived from these calculations may be employed as the identity of the amino acid sequences.

In the pyrophosphate phosphatase, the mutation may be introduced into sites in a catalytic domain and sites other than the catalytic domain as long as an objective activity is retained. The positions of amino acid residues to be mutated which are capable of retaining the objective activity are understood by a person skilled in the art. Specifically, a person skilled in the art can recognize a correlation between structure and function, since a person skilled in the art can 1) compare the amino acid sequences of multiple proteins having the same type of activity, 2) clarify regions that are relatively conserved and regions that are not relatively conserved, and then 3) predict regions capable of playing a functionally important role and regions incapable of playing a functionally important role from the regions that are relatively conserved and the regions that are not relatively conserved, respectively. Therefore, a person skilled in the art can identify the positions of the amino acid residues to be mutated in the amino acid sequence of the pyrophosphate phosphatase.

When the mutation of the amino acid residue is substitution, the substitution of the amino acid residue may be conservative substitution. The term “conservative substitution” refers to substitution of a certain amino acid residue with an amino acid residue having a similar side chain. Families of amino acid residues having similar side chains are well-known in the art. Examples of such families may include amino acids having a basic side chain (e.g., lysine, arginine, histidine), amino acids having an acidic side chain (e.g., aspartic acid, glutamic acid), amino acids having a non-charged polar side chain (e.g., asparagine, glutamine, serine, threonine, tyrosine, cysteine), amino acids having a non-polar side chain (e.g., glycine, alanine, valine, leucine, isoleucine, proline, phenylalanine, methionine, tryptophan), amino acids having a branched side chain at position f3 (e.g., threonine, valine, isoleucine), amino acids having an aromatic side chain (e.g., tyrosine, phenylalanine, tryptophan, histidine), amino acids having a hydroxyl group-containing (e.g., alcoholic, phenolic) side chain (e.g., serine, threonine, tyrosine), and amino acids having a sulfur-containing side chain (e.g., cysteine, methionine). Preferably, the conservative substitution of the amino acids may be the substitution between aspartic acid and glutamic acid, the substitution among arginine, lysine and histidine, the substitution between tryptophan and phenylalanine, the substitution between phenylalanine and valine, the substitution among leucine, isoleucine and alanine, and the substitution between glycine and alanine.

The expression of pyrophosphate phosphatase in the isoprene synthase-expressing microorganism can be improved by any mode in which an amount of pyrophosphate phosphatase expressed in the isoprene synthase-expressing microorganism is increased. Pyrophosphate phosphatase, the expression of which is to be improved in the isoprene synthase-expressing microorganism is pyrophosphate phosphatase that is homologous or heterologous to isoprene synthase and/or the isoprene synthase-expressing microorganism. Pyrophosphate phosphatase homologous to the isoprene synthase-expressing microorganism may be pyrophosphate phosphatase inherent to the isoprene synthase-expressing microorganism or foreign pyrophosphate phosphatase. One or a plurality (e.g., 2 or 3) of pyrophosphate phosphatase, the expression of which is to be improved in the isoprene synthase-expressing microorganism may be available.

The expression of pyrophosphate phosphatase in the isoprene synthase-expressing microorganism may be improved, for example, by modifying a surrounding region of a pyrophosphate phosphatase gene inherent to the isoprene synthase-expressing microorganism, or by transforming the isoprene synthase-expressing microorganism with a pyrophosphate phosphatase expression vector to introduce an expression unit comprising a polynucleotide encoding pyrophosphate phosphatase into the isoprene synthase-expressing microorganism. The expression vector used in the present invention may further comprise one or more regions that allow for homologous recombination with genome of a host cell when introduced into the host cell. For example, the expression vector may be designed such that an expression unit comprising a given polynucleotide is positioned between a pair of homologous regions (e.g., homology arm homologous to a certain sequence in host genome, loxP, FRT). The expression unit refers to a unit that comprises a given polynucleotide to be expressed and a promoter (homologous promoter, heterologous promoter) operably linked thereto and allows for transcription of the polynucleotide and further production of a polypeptide encoded by the polynucleotide. The expression unit may further comprise elements such as a terminator, a ribosome binding site and a drug resistant gene.

Examples of the expression vector used in the present invention may include vectors that expresses a protein in a host. The expression vector may also be a plasmid, a viral vector, a phage or an artificial chromosome. The expression vector may further be a DNA vector or an RNA vector. The expression vector may be an integrative vector or a non-integrative vector. The integrative vector may be a vector of a type where the vector is entirely integrated into genome of a host cell. Alternatively, the integrative vector may be a vector of a type where the vector is partially (e.g., the aforementioned expression unit) integrated into genome of a host cell.

Examples of the surrounding region to be modified in the pyrophosphate phosphatase gene may include a promoter region, Shine-Dalgarno (SD) sequence, and a spacer region between RBS and an initiation codon (in particular, a sequence just upstream of the initiation codon (5′-UTR)). Examples of the modification may include one or several (e.g., 1 to 500, 1 to 300, 1 to 200 or 1 to 100) nucleotide substitutions, insertions or deletions in the surrounding region. Preferably, the modification in the surrounding region is the substitution of the promoter region and if necessary the substitution of the SD sequence. Examples of a promoter to be introduced after the substitution may include inducible promoters such as a tac promoter (Ptac), a trc promoter (Ptrc) and a lac promoter (Plac). Examples of a sequence to be introduced after the substitution of the SD sequence may include RBS of a gene 10 derived from phage T7 (Olins P. O. et al, Gene, 1988, 73, 227-235, which is incorporated herein by reference in its entirety).

In the present invention, it is desirable that a copy number of the pyrophosphate phosphatase gene on a chromosome is increased thereby enhancing an activity. It is preferable that a plurality of copies, desirably 2 copies and more preferably 3 copies are carried on the chromosome. Increase of the copy number can be accomplished by introducing a plasmid carrying the pyrophosphate phosphatase gene into a host cell. The increase of the copy number can also be accomplished by utilizing transposon or Mu phage to transfer the pyrophosphate phosphatase gene onto the genome of the host.

The isoprene synthase-expressing microorganism is a microorganism that produces isoprene synthase. Preferably, the isoprene synthase-expressing microorganism is a microorganism obtained by transforming a host cell with an isoprene synthase-expressing vector to introduce an expression unit comprising a polynucleotide encoding the isoprene synthase into the host cell. The expression unit and the expression vector are as described above. It is preferable that a plurality of copies, desirably 2 copies and more preferably 3 copies of an isoprene synthase gene are carried on the chromosome in the isoprene synthase-expressing microorganism. Such an isoprene synthase-expressing microorganism can be obtained by introducing an isoprene synthase-expressing vector into a host. Also such an isoprene synthase-expressing microorganism can be obtained by utilizing transposon or Mu phage to transfer the isoprene synthase gene onto the genome of the host. The host cell may be homologous or heterologous to the isoprene synthase, but is preferably heterologous. Examples of the isoprene synthase gene contained in the isoprene synthase-expressing vector may include isoprene synthase genes derived from kudzu (Pueraria montana var. lobata), poplar (Populus alba x Populus tremula), Mucuna (Mucuna bracteata), willow (Salix), false acacia (Robinia pseudoacacia), wisteria (Wisterria), eucalyptus (Eucalyptus globulus), and tea plant (Melaleuca alterniflora) (see, e.g., Evolution 67 (4), 1026-1040 (2013) which is incorporated herein by reference in its entirety). The isoprene synthase-expressing vector may be an integrative vector or a non-integrative vector. A gene encoding the isoprene synthase can be arranged under control of a constitutive promoter or an inducible promoter (e.g., a promoter as described below which is inversely depending on the growth promoting agent) in the expression vector. Preferably, the gene encoding the isoprene synthase can be arranged under the control of the constitutive promoter. Examples of the constitutive promoter may include a tac promoter, a lac promoter, a trp promoter, a trc promoter, a T7 promoter, a T5 promoter, a T3 promoter, and an Sp6 promoter.

In one embodiment, the isoprene synthase may be, for example, a protein as follows:

1) a full-length protein which may be derived from Kudzu (the amino acid sequence of SEQ ID NO:8);

2) a protein obtained by deleting a chloroplast localization signal from the full-length protein in 1) above (amino acid sequence obtained by deleting amino acid residues at positions 1 to 45 in the amino acid sequence of SEQ ID NO:8);

3) a full-length protein which may be derived from Poplar (the amino acid sequence of SEQ ID NO:11);

4) a protein obtained by deleting a chloroplast localization signal from the full-length protein in 3) above (amino acid sequence obtained by deleting amino acid residues at positions 1 to 37 in the amino acid sequence of SEQ ID NO: 11);

5) a full-length protein which may be derived from Mucuna (the amino acid sequence of SEQ ID NO:7); and

6) a protein obtained by deleting a chloroplast localization signal from the full-length protein in 5) above (amino acid sequence obtained by deleting amino acid residues at positions 1 to 44 in the amino acid sequence of SEQ ID NO:7).

In a preferred embodiment, the isoprene synthase may be derived from Kudzu. In another preferred embodiment, the isoprene synthase may be derived from Poplar. In still another preferred embodiment, the isoprene synthase may be derived from Mucuna.

In another embodiment, the isoprene synthase is a protein that comprises an amino acid sequence having 70% or more amino acid sequence identity to the amino acid sequence of the proteins of 1) to 6) above, and has an isoprene synthase activity. The amino acid sequence percent identity may be, for example, 75% or more, 80% or more, 85% or more, 90% or more, 95% or more, 96% or more, 97% or more, 98% or more, or 99% or more. The amino acid sequence percent identity can be determined in the aforementioned manner. The isoprene synthase activity refers to an activity of forming isoprene from dimethylallyl diphosphate (DMAPP).

In still another embodiment, the isoprene synthase is a protein that comprises an amino acid sequence having a mutation of one or several amino residues in the amino acid sequence of the protein of 1) to 6) above, and has an isoprene synthase activity. Examples of the mutation of the amino acid residues may include deletion, substitution, addition and insertion of amino acid residues. The mutation of one or several amino acid residues may be introduced into one region or multiple different regions in the amino acid sequence. The term “one or several” indicates a range in which a three-dimensional structure and an activity of the protein are not impaired greatly. In the case of the protein, the number represented by “one or several” is, for example, 1 to 100, preferably 1 to 80, more preferably 1 to 50, 1 to 30, 1 to 20, 1 to 10, or 1 to 5.

The isoprene synthase preferably has an isoprene synthase activity that is 50% or more, 60% or more, 70% or more, 80% or more, 90% or more, or 95% or more of the isoprene synthase activity of the protein of 1) to 6) above when measured under the same conditions (e.g., buffer, concentration, temperature, and reaction time). In terms of stability, it is also preferable that the isoprene synthase has a remaining activity that is 30% or more, 40% or more, 50% or more, 60% or more or 65% or more of the original activity when stored in a certain buffer (e.g., a solution of 50 mM Tris-HCl (pH 8.0), and 0.15 mM MgCl₂) at 4° C. for 48 hours.

In the isoprene synthase, the mutation may be introduced into sites in a catalytic domain and sites other than the catalytic domain as long as an objective activity is retained. The positions of amino acid residues to be mutated which are capable of retaining the objective activity are understood by a person skilled in the art. Specifically, a person skilled in the art can recognize a correlation between structure and function, since a person skilled in the art can 1) compare the amino acid sequences of multiple proteins having the same type of activity, 2) clarify regions that are relatively conserved and regions that are not relatively conserved, and then 3) predict regions capable of playing a functionally important role and regions incapable of playing a functionally important role from the regions that are relatively conserved and the regions that are not relatively conserved, respectively. Therefore, a person skilled in the art can identify the positions of the amino acid residues to be mutated in the amino acid sequence of the isoprene synthase. When an amino acid residue is mutated by substitution, the substitution of the amino acid residue may be the conservative substitution as described above.

Preferably, the isoprene synthase-expressing microorganism may be a microorganism that further expresses a mevalonate kinase in addition to the isoprene synthase. Therefore, in the isoprene synthase-expressing microorganism, a mevalonate kinase expression vector may be introduced into a host. Examples of the mevalonate kinase gene to be introduced into the host by the mevalonate kinase expression vector may include genes from microorganisms belonging to the genus Methanosarcina such as Methanosarcina mazei, the genus Methanocella such as Methanocella paludicola, the genus Corynebacterium such as Corynebacterium variabile, the genus Methanosaeta such as Methanosaeta concilii, and the genus Nitrosopumilus such as Nitrosopumilus maritimus. The mevalonate kinase expression vector may be an integrative vector or a non-integrative vector. In the expression vector, the gene encoding the mevalonate kinase may be placed under the control of the constitutive promoter as described above or an inducible promoter (e.g., a promoter as described below which is inversely dependent on the growth promoting agent). Preferably, the gene encoding the mevalonate kinase may be placed under the control of the constitutive promoter.

For the isoprene synthase-expressing microorganism (host cell) used in the present invention, a bacterium or a fungus is preferred. The bacterium may be a gram-positive bacterium or a gram-negative bacterium. For the isoprene synthase-expressing microorganism, a microorganism belonging to the family Enterobacteriaceae, in particular, a microorganism belonging to the family Enterobacteriaceae among microorganisms as described below, is also preferred.

Examples of the gram-positive bacterium may include bacteria belonging to the genera Bacillus, Listeria, Staphylococcus, Streptococcus, Enterococcus, Clostridium, Corynebacterium, and Streptomyces. Bacteria belonging to the genera Bacillus and Corynebacterium are preferable.

Examples of the bacteria belonging to the genus Bacillus may include Bacillus subtilis, Bacillus anthracis, and Bacillus cereus. Bacillus subtilis is more preferable.

Examples of the bacteria belonging to genus the Corynebacterium may include Corynebacterium glutamicum, Corynebacterium efficiens, and Corynebacterium callunae. Corynebacterium glutamicum is more preferable.

Examples of the gram-negative bacterium may include bacteria belonging to the genera Escherichia, Pantoea, Salmonella, Vivrio, Serratia, and Enterobacter. The bacteria belonging to the genera Escherichia, Pantoea and Enterobacter are preferable.

Escherichia coli is preferable as the bacteria belonging to the genus Escherichia.

Examples of the bacteria belonging to the genus Pantoea may include Pantoea ananatis, Pantoea stewartii, Pantoea agglomerans, and Pantoea citrea. Pantoea ananatis and Pantoea citrea are preferable. Strains exemplified in EP 0 952 221, which is incorporated herein by reference in its entirety, may be used as the bacteria belonging to the genus Pantoea. Examples of representative strains of the bacteria belonging to genus Pantoea may include Pantoea ananatis AJ13355 strain (FERM BP-6614) and Pantoea ananatis AJ13356 strain (FERM BP-6615), both of which are disclosed in EP 0 952 221, which is incorporated herein by reference in its entirety, and Pantoea ananatis SC17(0) strain. SC17(0) was deposited to Russian National Collection of Industrial Microorganisms (VKPM), GNII Genetika (address: Russia, 117545 Moscow, 1 Dorozhny proezd. 1) as of Sep. 21, 2005, with the deposit number of VKPM B-9246.

Examples of the bacteria belonging to the genus Enterobacter may include Enterobacter agglomerans and Enterobacter aerogenes. Enterobacter aerogenes is preferable. The bacterial strains exemplified in EP 0 952 221, which is incorporated herein by reference in its entirety, may be used as the bacteria belonging to the genus Enterobacter. Examples of representative strains of the bacteria belonging to the genus Enterobacter may include Enterobacter agglomerans ATCC12287 strain, Enterobacter aerogenes TACC13048 strain, Enterobacter aerogenes NBRC12010 strain (Biotechnol. Bioeng., 2007 Mar. 27; 98(2): 340-348, which is incorporated herein by reference inits entirety), and Enterobacter aerogenes AJ110637 (FERM BP-10955). The Enterobacter aerogenes AJ110637 strain was deposited to International Patent Organism Depositary (IPOD), National Institute of Advanced Industrial Science and Technology (AIST) (Chuo No. 6, Higashi 1-1-1, Tsukuba City, Ibaraki Pref., JP, Postal code 305-8566) as of Aug. 22, 2007, with the deposit number of FERM P-21348 and was transferred to the international deposition based on Budapest Treaty on Mar. 13, 2008, and the receipt number FERM BP-10955 was given thereto.

Examples of the fungus may include microorganisms belonging to the genera Saccharomyces, Schizosaccharomyces, Yarrowia, Trichoderma, Aspergillus, Fusarium, and Mucor. The microorganisms belonging to the genera Saccharomyces, Schizosaccharomyces, Yarrowia, or Trichoderma are preferable.

Examples of the microorganisms belonging to the genus Saccharomyces may include Saccharomyces carlsbergensis, Saccharomyces cerevisiae, Saccharomyces diastaticus, Saccharomyces douglasii, Saccharomyces kluyveri, Saccharomyces norbensis, and Saccharomyces oviformis. Saccharomyces cerevisiae is preferable.

Schizosaccharomyces pombe is preferable as a microorganism belonging to the genus Schizosaccharomyces.

Yarrowia lypolytica is preferable as a microorganism belonging to the genus Yarrowia.

Examples of the microorganisms belonging to the genus Trichoderma may include Trichoderma harzianum, Trichoderma koningii, Trichoderma longibrachiatum, Trichoderma reesei, and Trichoderma viride. Trichoderma reesei is preferable.

In the isoprene synthase-expressing microorganism of the present invention, the pathway to synthesize dimethylallyl diphosphate (DMAPP) that is the substrate of the isoprene synthase may be further enhanced. For such an enhancement, an expression vector that expresses an isopentenyl-diphosphate delta isomerase having an ability to convert isopentenyl diphosphate (IPP) into dimethylallyl diphosphate (DMAPP) may be introduced into the isoprene synthase-expressing microorganism of the present invention. An expression vector that expresses one or more enzymes involved in the mevalonate pathway and/or methylerythritol phosphate pathway associated with formation of IPP and/or DMAPP may also be introduced into the isoprene synthase-expressing microorganism of the present invention. The expression vector for such an enzyme may be an integrative vector or a non-integrative vector. The expression vector for such an enzyme may further express at a time or separately a plurality of enzymes (e.g., one, two, three or four or more) involved in the mevalonate pathway and/or the methylerythritol phosphate pathway, and may be, for example, an expression vector for polycistronic mRNA. The origin of one or more enzymes involved in the mevalonate pathway and/or the methylerythritol phosphate pathway may be homologous or heterologous to the host. When the origin of the enzyme involved in the mevalonate pathway and/or the methylerythritol phosphate pathway is heterologous to the host, for example, the host may be a bacterium as described above (e.g., Escherichia coli) and the enzyme involved in the mevalonate pathway may be derived from a fungus (e.g., Saccharomyces cerevisiae). In addition, when the host inherently produces the enzyme involved in the methylerythritol phosphate pathway, an expression vector to be introduced into the host may express an enzyme involved in the mevalonate pathway.

Examples of isopentenyl-diphosphate delta isomerase (EC: 5.3.3.2) may include Idi1p (ACCESSION ID NP_015208), AT3G02780 (ACCESSION ID NP_186927), AT5G16440 (ACCESSION ID NP_197148) and Idi (ACCESSION ID NP_417365). In the expression vector, a gene encoding an isopentenyl-diphosphate delta isomerase may be placed under the control of a promoter as described below which is inversely dependent on a growth promoting agent.

Examples of the enzymes involved in the mevalonate (MVA) pathway may include mevalonate kinase (EC: 2.7.1.36; example 1, Erg12p, ACCESSION ID NP_013935; example 2, AT5G27450, ACCESSION ID NP_001190411), phosphomevalonate kinase (EC: 2.7.4.2; example 1, Erg8p, ACCESSION ID NP_013947; example 2, AT1G31910, ACCESSION ID NP_001185124), diphosphomevalonate decarboxylase (EC: 4.1.1.33; example 1, Mvd1p, ACCESSION ID NP_014441; example 2, AT2G38700, ACCESSION ID NP_181404; example 3, AT3G54250, ACCESSION ID NP_566995), acetyl-CoA-C-acetyltransferase (EC: 2.3.1.9; example 1, Erg10p, ACCESSION ID NP_015297; example 2, AT5G47720, ACCESSION ID NP_001032028; example 3, AT5G48230, ACCESSION ID NP_568694), hydroxymethylglutaryl-CoA synthase (EC: 2.3.3.10; example 1, Erg13p, ACCESSION ID NP_013580; example 2, AT4G11820, ACCESSION ID NP_192919; example 3, MvaS, ACCESSION ID AAG02438), hydroxymethylglutaryl-CoA reductase (EC: 1.1.1.34; example 1, Hmg2p, ACCESSION ID NP_013555; example 2, Hmg1p, ACCESSION ID NP_013636; example 3, AT1G76490, ACCESSION ID NP_177775; example 4, AT2G17370, ACCESSION ID NP_179329, EC: 1.1.1.88, example, MvaA, ACCESSION ID P13702), and acetyl-CoA-C-acetyltransferase/hydroxymethylglutaryl-CoA reductase (EC: 2.3.1.9/1.1.1.34, example, MvaE, ACCESSION ID AAG02439). In the expression vector, a gene encoding one or more enzymes involved in the mevalonate (MVA) pathway (e.g., phosphomevalonate kinase, diphosphomevalonate decarboxylase, acetyl-CoA-C-acetyltransferase/hydroxymethylglutaryl-CoA reductase, hydroxymethylglutaryl-CoA synthase) may be placed under the control of a promoter as described below which is inversely dependent on the growth promoting agent.

Examples of the enzymes involved in the methylerythritol phosphate (MEP) pathway may include 1-deoxy-D-xylulose-5-phosphate synthase (EC: 2.2.1.7, example 1, Dxs, ACCESSION ID NP_414954; example 2, AT3G21500, ACCESSION ID NP_566686; example 3, AT4G15560, ACCESSION ID NP_193291; example 4, AT5G11380, ACCESSION ID NP_001078570), 1-deoxy-D-xylulose-5-phosphate reductoisomerase (EC: 1.1.1.267; example 1, Dxr, ACCESSION ID NP_414715; example 2, AT5G62790, ACCESSION ID NP_001190600), 4-diphosphocytidyl-2-C-methyl-D-erythritol synthase (EC: 2.7.7.60; example 1, IspD, ACCESSION ID NP_417227; example 2, AT2G02500, ACCESSION ID NP_565286), 4-diphosphocytidyl-2-C-methyl-D-erythritol kinase (EC: 2.7.1.148; example 1, IspE, ACCESSION ID NP_415726; example 2, AT2G26930, ACCESSION ID NP_180261), 2-C-methyl-D-erythritol-2,4-cyclodiphosphate synthase (EC: 4.6.1.12; example 1, IspF, ACCESSION ID NP_417226; example 2, AT1G63970, ACCESSION ID NP_564819), 1-hydroxy-2-methyl-2-(E)-butenyl-4-diphosphate synthase (EC: 1.17.7.1; example 1, IspG, ACCESSION ID NP_417010; example 2, AT5G60600, ACCESSION ID NP_001119467), and 4-hydroxy-3-methyl-2-butenyl diphosphate reductase (EC: 1.17.1.2; example 1, IspH, ACCESSION ID NP_414570; example 2, AT4G34350, ACCESSION ID NP_567965). In the expression vector, a gene encoding one or more enzymes involved in the methylerythritol phosphate (MEP) pathway may be placed under the control of a promoter as described below which is inversely dependent on the growth promoting agent.

Transformation of the host cell by the expression vector in which the gene is incorporated can be carried out using known methods. Examples of such a method may include a competent cell method using a microbial cell treated with calcium and an electroporation method. The gene may be introduced by infecting the microbial cell with a phage vector rather than the plasmid vector.

Further, a gene encoding the enzyme involved in the mevalonate pathway or the methylerythritol phosphate pathway that synthesizes dimethylallyl diphosphate that is the substrate of the isoprene synthase may also be introduced into the isoprene synthase-expressing microorganism of the present invention. Examples of such an enzyme may include 1-deoxy-D-xylose-5-phosphate synthase that converts a pyruvate and D-glycelaldehyde-3-phosphate into 1-deoxy-D-xylose-5-phosphate, and isopentyl diphosphate isomerase that converts isopentenyl diphosphate into dimethylallyl diphosphate. In the expression vector, a gene encoding the enzyme involved in the mevalonate pathway or the methylerythritol phosphate pathway that synthesizes dimethylallyl diphosphate may be placed under the control of the constitutive promoters as described above or an inducible promoter (e.g., a promoter as described below, which is inversely dependent on the growth promoting agent).

DMAPP (dimethylallyl diphosphate) that is a substrate of isoprene synthesis has been known to be a precursor of peptide glycan and an electron acceptor, such as menaquinone and the like, and to be essential for growth of microorganisms (Fujisaki et al., J. Biochem., 1986; 99: 1137-1146, which is incorporated herein by reference in its entirety). In view of efficient production of isoprene, the isoprene synthase-expressing microorganism of the present invention may be used in the method of producing isoprene, in which a step 1) corresponding to a growth phase of a microorganism (culturing an isoprene-expressing microorganism in the presence of a growth promoting agent at a sufficient concentration to grow the isoprene-expressing microorganism) and a step 3) corresponding to a formation phase of the isoprene (culturing the isoprene-expressing microorganism to form an isoprene monomer) are separated. The method may also comprise a step 2) corresponding to an induction phase of isoprene production for transferring the growth phase of the microorganism to the formation phase of the isoprene (decreasing the sufficient concentration of the growth promoting agent to induce production of the isoprene monomer by the isoprene-expressing microorganism).

In the method of the present invention, the growth promoting agent can refer to a factor essential for the growth of a microorganism or a factor having an activity of promoting the growth of the microorganism, which can be consumed by the microorganism, the consumption of which causes a reduction of its amount in the culture medium, consequently loss or reduction of the growth of the microorganism. For example, when the growth promoting agent in a certain amount is used, a microorganism continues to grow until the growth promoting agent in that amount is consumed, but once the growth promoting agent is entirely consumed, the microorganism cannot grow or the growth rate can decrease. Therefore, the degree of the growth of the microorganism can be regulated by the growth promoting agent. Examples of such a growth promoting agent may include substances such as oxygen (gas); minerals such as ions of iron, magnesium, potassium and calcium; phosphorus compounds such as monophosphoric acid, diphosphoric acid and polyphosphoric acid, or salt thereof; nitrogen compounds (gas) such as ammonia, nitrate, nitrite, and urea; sulfur compounds such as ammonium sulfate and thiosulfuric acid; and nutrients such as vitamins (e.g., vitamin A, vitamin D, vitamin E, vitamin K, vitamin B1, vitamin B2, vitamin B6, vitamin B12, niacin, pantothenic acid, biotin, ascorbic acid), and amino acids (e.g., alanine, arginine, asparagine, aspartic acid, cysteine, glutamine, glutamic acid, glycine, histidine, leucine, isoleucine, lysine, methionine, phenylalanine, proline, serine, threonine, tryptophan, tyrosine, valine, selenocysteine). One growth promoting agent may be used or two or more growth promoting agents may be used in combination in the method of the present invention.

When the growth promoting agent is used, the isoprene-expressing microorganism of the present invention may have an ability to grow depending on the growth promoting agent and an ability to form isoprene depending on a promoter which is inversely dependent on the growth promoting agent. An isoprene-producing microorganism can grow in the presence of the growth promoting agent at concentration sufficient for the growth of the isoprene-producing microorganism. Here, the “sufficient concentration” can refer to that the growth promoting agent is used at concentration which is effective for the growth of the isoprene-producing microorganism. The expression “ability to produce an(the) isoprene depending on a promoter which is inversely depending on the growth promoting agent” can mean that the isoprene cannot be produced or a producing efficiency of the isoprene is low in the presence of the growth promoting agent at relatively high concentration whereas the isoprene can be produced or the producing efficiency of the isoprene is high in the presence of the growth promoting agent at relatively low concentration or in the absence of the growth promoting agent. Therefore, the isoprene-producing microorganism used in the present invention can grow well but cannot produce the isoprene or exhibits low producing efficiency of the isoprene in the presence of the growth promoting agent at sufficient concentration. The isoprene-producing microorganism cannot grow well but can produce the isoprene and exhibits high producing efficiency of the isoprene in the presence of the growth promoting agent at insufficient concentration or in the absence of the growth promoting agent.

In such an isoprene-producing microorganism, a gene encoding the above-described enzyme can be present under the control of a promoter which is inversely dependent on the growth promoting agent. The expression “promoter which is inversely dependent on the growth promoting agent” can mean a promoter not having at all or having low transcription activity in the presence of the growth promoting agent at relatively high concentration but having some or high transcription activity in the presence of the growth promoting agent at relatively low concentration or in the absence of the growth promoting agent. Therefore, the promoter which is inversely dependent on the growth promoting agent can suppress the expression of the gene encoding the above-described enzyme in the presence of the growth promoting agent at a concentration sufficient for the growth of the isoprene-producing microorganism whereas it can promote the expression of the gene encoding the above-described enzyme in the presence of the growth promoting agent at the concentration insufficient for the growth of the isoprene-producing microorganism or in the absence of the growth promoting agent. Preferably, the isoprene-producing microorganism is a microorganism transformed with an expression vector comprising the gene encoding the above-described enzyme under the control of the promoter which is inversely dependent on the growth promoting agent.

For example, when the growth promoting agent is oxygen, a microaerobically inducible promoter can be utilized. The microaerobically inducible promoter can refer to a promoter that can promote the expression of a downstream gene under a microaerophilic condition. In general, the saturated concentration of dissolved oxygen is 7.22 ppm (under the air condition: 760 mmHg, 33° C., 20.9% oxygen and saturated water vapor). The microaerophilic condition can refer to a condition where a (dissolved) oxygen concentration is 0.35 ppm or less. The (dissolved) oxygen concentration under the microaerophilic condition may be 0.30 ppm or less, 0.25 ppm or less, 0.20 ppm or less, 0.15 ppm or less, 0.10 ppm or less, or 0.05 ppm or less. Examples of the microaerobically inducible promoter may include a promoter of the gene encoding a D- or L-lactate dehydrogenase (e.g., lld, ldhA), a promoter of the gene encoding an alcohol dehydrogenase (e.g., adhE), a promoter of the gene encoding a pyruvate formate lyase (e.g., pflB), and a promoter of the gene encoding an α-acetolactate decarboxylase (e.g., budA).

When the growth promoting agent is a phosphorus compound, a phosphorus deficiency-inducible promoter can be utilized. The expression “phosphorus deficiency-inducible promoter” can refer to a promoter that can promote the expression of a downstream gene at low concentration of phosphorus compound. The low concentration of phosphorus compound can refer to a condition where a (free) phosphorus concentration is 100 mg/L or less. The expression “phosphorus” is synonymous to the expression “phosphorus compound”, and they can be used in exchangeable manner. The (free) phosphorus concentration under a phosphorus deficient condition may be 50 mg/L or less, 10 mg/L or less, 5 mg/L or less, 1 mg/L or less, 0.1 mg/L or less, or 0.01 mg/L or less. Examples of the phosphorus deficiency-inducible promoter may include a promoter of the gene encoding alkali phosphatase (e.g., phoA), a promoter of the gene encoding an acid phosphatase (e.g., phoC), a promoter of the gene encoding a sensor histidine kinase (phoR), a promoter of the gene encoding a response regulator (e.g., phoB), and a promoter of the gene encoding a phosphorus uptake carrier (e.g., pstS).

When the growth promoting agent is an amino acid, an amino acid deficiency-inducible promoter can be utilized. The amino acid deficiency-inducible promoter can refer to a promoter that can promote the expression of a downstream gene at low concentration of an amino acid. The low concentration of the amino acid can refer to a condition where a concentration of a (free) amino acid or a salt thereof is 100 mg/L or less. The concentration of the (free) amino acid or a salt thereof under the amino acid deficient condition may be 50 mg/L or less, 10 mg/L or less, 5 mg/L or less, 1 mg/L or less, 0.1 mg or less or 0.01 mg/L or less. Examples of the amino acid deficiency-inducible promoter may include a promoter of the gene encoding a tryptophan leader peptide (e.g., trpL) and a promoter of the gene encoding an N-acetylglutamate synthase (e.g., ArgA).

### Method of Producing Isoprene Monomer and Isoprene Polymer.

The present invention provides a method of producing an isoprene monomer. The method of producing an isoprene monomer of the present invention includes culturing an isoprene synthase-expressing microorganism in a culture medium so as to form an isoprene monomer.

The method of producing the isoprene monomer of the present invention can be performed by culturing the isoprene synthase-expressing microorganism of the present invention. Dimethylallyl diphosphate that is a raw material of the isoprene monomer is efficiently supplied from a carbon source in a culture medium by the isoprene synthase-expressing microorganism of the present invention. The isoprene synthase-expressing microorganism of the present invention produces the isoprene monomer mainly as an outgas from the carbon source in the culture medium. Thus, the isoprene monomer is recovered by collecting gas produced from the transformant. Dimethylallyl diphosphate that is the substrate of the isoprene synthase is synthesized from the carbon source in the culture medium via the mevalonate pathway or the methylerythritol phosphate pathway in the host cells.

The culture medium for culturing the isoprene synthase-expressing microorganism of the present invention preferably contains the carbon source to be converted into isoprene. The carbon source may include carbohydrates such as monosaccharides, disaccharides, oligosaccharides and polysaccharides; invert sugars obtained by hydrolyzing sucrose; glycerol; compounds having one carbon atom (hereinafter referred to as a C1 compound) such as methanol, formaldehyde, formate, carbon monoxide and carbon dioxide; oils such as corn oil, palm oil and soybean oil; acetate; animal fats; animal oils; fatty acids such as saturated fatty acids and unsaturated fatty acids; lipids; phospholipids; glycerolipids; glycerine fatty acid esters such as monoglyceride, diglyceride and triglyceride; polypeptides such as microbial proteins and plant proteins; renewable carbon sources such as hydrolyzed biomass carbon sources; yeast extracts, or combinations thereof. For a nitrogen source, inorganic ammonium salts such as ammonium sulfate, ammonium chloride and ammonium phosphate, organic nitrogen such as hydrolyzed soybeans, ammonia gas, ammonia water, and the like can be used. It is desirable to include required substances such as vitamin B1 and L-homoserine, or yeast extract and the like in an appropriate amount as an organic trace nutrient source. In addition thereto, potassium phosphate, magnesium sulfate, iron ion, manganese ion, and the like may be added in small amounts if necessary. The culture medium used in the present invention may be a natural medium or a synthesized medium as long as the culture medium contains a carbon source, a nitrogen source, inorganic ions, and optionally other organic trace ingredients.

Examples of the monosaccharides may include triose such as ketotriose (dihydroxyacetone) and aldotriose (glyceraldehyde); tetrose such as ketotetrose (erythrulose) and aldotetrose (erythrose, threose); pentose such as ketopentose (ribulose, xylulose), aldopentose (ribose, arabinose, xylose, lyxose) and deoxysaccharide (deoxyribose); hexose such as ketohexose (psychose, fructose, sorbose, tagatose), aldohexose (allose, altrose, glucose, mannose, gulose, idose, galactose, tallose), and deoxysaccharide (fucose, fucrose, rhamnose); and heptose such as sedoheptulose. C6 sugars such as fructose, mannose, galactose and glucose; and C5 sugars such as xylose and arabinose are preferable.

Examples of the disaccharides may include sucrose, lactose, maltose, trehalose, turanose, and cellobiose. Sucrose and lactose are preferable.

Examples of the oligosaccharides may include trisaccharides such as raffinose, melezitose and maltotriose; tetrasaccharides such as acarbose and stachyose; and other oligosaccharides such as fructooligosaccharide (FOS), galactooligosaccharide (GOS) and mannan-oligosaccharide (MOS).

Examples of the polysaccharides may include glycogen, starch (amylose, amylopectin), cellulose, dextrin, and glucan (β1,3-glucan). Starch and cellulose are preferable.

Examples of the microbial protein may include polypeptides obtainable from a yeast or bacterium. Examples of the plant protein may include polypeptides obtainable from soybean, corn, canola, Jatropha, palm, peanut, sunflower, coconut, mustard, cotton seed, palm kernel oil, olive, safflower, sesame and linseed.

Examples of the lipid may include substances containing one or more saturated or unsaturated fatty acids of C4 or more.

The oil is preferably the lipid that contains one or more saturated or unsaturated fatty acids of C4 or more and is liquid at room temperature, and examples of the oil may include lipids obtainable from soybean, corn, canola, Jatropha, palm, peanut, sunflower, coconut, mustard, cotton seed, Palm kernel oil, olive, safflower, sesame, linseed, oily microbial cells, Chinese tallow tree, and a combination of two or more thereof.

Examples of the fatty acid may include compounds represented by a formula RCOOH (“R” represents a hydrocarbon group).

The unsaturated fatty acid is a compound having at least one double bond between two carbon atoms in “R”, and examples of the unsaturated fatty acid may include oleic acid, vaccenic acid, linoleic acid, palmitelaidic acid and arachidonic acid.

The saturated fatty acid is a compound where the “R” is a saturated aliphatic group, and examples of the saturated fatty acid may include docosanoic acid, eicosanoic acid, octadecanoic acid, hexadecanoic acid, tetradecanoic acid, and dodecanoic acid.

Among them, those containing one or more C2 to C22 fatty acids are preferable as the fatty acid, and those containing C12 fatty acid, C14 fatty acid, C16 fatty acid, C18 fatty acid, C20 fatty acid and C22 fatty acid are more preferable.

The carbon source may include salts and derivatives of these fatty acids and salts of these derivatives. Examples of the salt may include lithium salts, potassium salts and sodium salts.

Examples of the carbon source may also include combinations of carbohydrate such as glucose with the lipid(s), the oil(s), the fats, the fatty acid(s) and glycerin fatty acid(s) ester(s).

Examples of the renewable carbon source may include hydrolyzed biomass carbon sources.

Examples of the biomass carbon source may include cellulose-based substrates such as waste materials of woods, papers and pulps, leafy plants, and fruit pulps; and partial plants such as stalks, grain particles, roots and tubers.

Examples of the plants to be used as the biomass carbon source may include corn, wheat, rye, sorghum, triticale, rice, millet, barley, cassava, legumes such as peas, potato, sweet potato, banana, sugar cane, and tapioca.

When the renewable carbon source such as biomass is added to the culture medium, the carbon source is preferably pretreated. Examples of the pretreatment may include an enzymatic pretreatment, a chemical pretreatment, and a combination of the enzymatic pretreatment and the chemical pretreatment.

It is preferred that the renewable carbon source is entirely or partially hydrolyzed before being added to the culture medium.

Examples of the carbon source may also include the yeast extract and a combination of the yeast extract with the other carbon source such as glucose. The combination of the yeast extract with the C1 compound such as carbon dioxide and methanol is preferable.

In the method of culturing the transformant according to the present invention, it is preferable the cell is cultured in a standard medium containing saline and nutrients.

The culture medium is not particularly limited, and examples of the culture medium may include ready-made general media that are commercially available such as Luria Bertani (LB) broth, Sabouraud dextrose (SD) broth, and yeast medium (YM) broth. The medium suitable for the cultivation of the specific host can be selected appropriately for the use.

It is desirable to include appropriate minerals, salts, supplemental elements, buffers, and ingredients known for those skilled in the art to be suitable for the cultivation and to facilitate the production of isoprene in addition to the appropriate carbon source in the cell medium.

A culture condition for the isoprene synthase-expressing microorganism of the present invention is not particularly limited as long as the isoprene formation ability by the isoprene synthase-expressing microorganism can be improved as a result of enhancement in the expression of the pyrophosphate phosphatase, and a standard cell culture condition can be used.

The culture temperature is preferably 20 to 37° C., the gas composition is preferably about 6 to about 84% of CO₂ concentration, and the pH value is preferably about 5 to about 9.

It is preferable that the culturing is performed under an aerobic, oxygen-free, or anaerobic condition depending on a nature of the host cell.

Examples of methods of culturing the transformant include a method using a known fermentation method such as a batch cultivation method, a feeding cultivation method or a continuous cultivation method.

In the batch cultivation method, a medium composition is added at start of the fermentation, the host cell is inoculated in the medium composition and the transformant is cultured while pH and an oxygen concentration are controlled.

In the cultivation of the transformant by the batch cultivation method, the growth of the transformant starts from a mild induction phase, passes through a logarithmic growth phase and finally goes to a stationary phase in which a growth speed is reduced or stopped. Isoprene is produced by the transformant in the logarithmic growth phase and the stationary phase.

In the feeding cultivation method, in addition to the above batch method, the carbon source is gradually added according to the progress of a fermentation process. The feeding cultivation method is effective when an amount of the carbon source is to be restricted in the medium because metabolism of the transformant tends to be reduced due to catabolite suppression. The feed cultivation can be performed using a restricted amount or an excessive amount of the carbon source such as glucose.

In the continuous cultivation method, a certain amount of the medium is continuously supplied to a bioreactor at a constant rate while the same amount of the medium is removed. In the continuous cultivation method, the culture can be kept constantly at high concentration and the transformant in the culture medium is generally in the logarithmic growth phase.

The nutrition can be supplemented by entirely or partly exchanging the medium appropriately, and accumulation of metabolic byproducts that potentially have adverse effects on the growth of the transformant, and the accumulation of dead cells can be prevented.

Examples of the promoter possessed by the expression vector to be introduced into the isoprene synthase-expressing microorganism of the present invention may include the promoters as described above. When the expression vector to be introduced into the isoprene synthase-expressing microorganism of the present invention has the inducible promoter such as a lac promoter, the expression of protein may be induced by, for example, adding IPTG (isopropyl-β-thiogalactopyranoside) into the culture medium.

Examples of the method of evaluating the amount of isoprene monomer produced by culturing the isoprene synthase-expressing microorganism of the present invention may include a method in which a gas phase is collected by a headspace method and this gas phase is analyzed by gas chromatography.

In detail, the isoprene monomer in a headspace which is obtained by culturing the transformant in a sealed vial with shaking the culture medium is analyzed by standard gas chromatography. Then, an area calculated by a curve measured by gas chromatography is converted into the amount of the isoprene monomer produced with the transformant using a standard curve.

Examples of the method of collecting the isoprene monomer obtained by culturing the isoprene synthase-expressing microorganism of the present invention may include gas stripping, fractional distillation, or dissociation of the isoprene monomer adsorbed to a solid phase by heat or vacuum, or extraction with a solvent.

In the gas stripping, isoprene gas is continuously removed from the outgas. Such removal of the isoprene gas can be performed by various methods. Examples of the removal may include adsorption to the solid phase, separation into a liquid phase, and a method in which the isoprene gas is directly condensed.

The isoprene monomer can be collected by a single step or multiple steps. When the isoprene monomer is collected by the single step, the isoprene monomer is converted into the liquid phase simultaneously with separating the isoprene monomer from the outgas. The isoprene monomer can also be directly condensed from the outgas to make the liquid phase. When the isoprene monomer is collected by the multiple stages, the isoprene monomer is separated from off-gas and subsequently converted into the liquid phase. For example, the isoprene monomer is adsorbed to the solid phase, and extracted from the solid phase with the solvent.

Exemplary methods of collecting the isoprene monomer may comprise further purifying the isoprene monomer. Examples of the purification may include separation from a liquid phase extract by distillation and various chromatographic methods.

The present invention provides further a method of producing an isoprene polymer. The method of producing the isoprene polymer according to the present invention comprises the following (I) and (II):

(I) producing an isoprene monomer by the method of the present invention; and

(II) polymerizing the isoprene monomer to form an isoprene polymer.

The step (I) can be performed in the same manner as in the method of producing the isoprene monomer according to the present invention described above. The polymerization of the isoprene monomer in the step (II) can be performed by any method such as addition polymerization known in the art (e.g., synthesis methods in organic chemistry).

The rubber composition of the present invention comprises a polymer derived from isoprene produced by a method for producing isoprene according to the present invention. The polymer derived from isoprene may be a homopolymer (i.e., isoprene polymer) or a heteropolymer comprising isoprene and one or more monomer units other than the isoprene (e.g., a copolymer such as a block copolymer). Preferably, the polymer derived from isoprene is a homopolymer (i.e., isoprene polymer) produced by a method for producing isoprene polymer according to the present invention. The rubber composition of the present invention may further comprise one or more polymers other than the above polymer, one or more rubber components, and/or other components. The rubber composition of the present invention can be manufactured using a polymer derived from isoprene. For example, the rubber composition of the present invention can be prepared by mixing a polymer derived from isoprene with one or more polymers other than the above polymer, one or more rubber components, and/or other components such as a reinforcing filler, a crosslinking agent, a vulcanization accelerator and an antioxidant.

The tire of the present invention is manufactured using the rubber composition of the present invention. The rubber composition of the present invention may be applied to any portion of the tire without limitation, which may be selected as appropriate depending on the application thereof. For example, the rubber composition of the present invention may be used in a tread, a base tread, a sidewall, a side reinforcing rubber and a bead filler of a tire. The tire can be manufactured by a conventional method. For example, a carcass layer, a belt layer, a tread layer, which are composed of unvulcanized rubber, and other members used for the production of usual tires may be successively laminated on a tire molding drum, then the drum may be withdrawn to obtain a green tire. Thereafter, the green tire may be heated and vulcanized in accordance with an ordinary method, to thereby obtain a desired tire (e.g., a pneumatic tire).

Other features of the invention will become apparent in the course of the following descriptions of exemplary embodiments which are given for illustration of the invention and are not intended to be limiting thereof.

### EXAMPLES

### Example 1

### Enhancement of ppa Gene Expression in MG1655 Ptac-KKDyI Strain

A Strain in which a Promoter Inherent to an Endogenous Ppa Gene (Pyrophosphate phosphatase gene) was substituted with another strong promoter to augment the expression of the endogenous ppa gene in E. coli strain was made by the following procedure.

First, competent cells of MG1655 Ptac-KKDyI strain (s Reference Example 7-4; this strain is a transformant of E. coli) for electroporation were prepared as follows. Cells of MG1655 Ptac-KKDyI strain were cultured with shaking in 5 mL of LB medium at 37° C. overnight. Subsequently, 50 λL, of the resulting cultured medium was inoculated to new 5 mL LB medium and cultured with shaking at 37° C. until absorbance at OD600 became around 0.6. Then, the microbial cells were collected, washed three times with ice-cooled 10% glycerol, and finally suspended in 0.5 mL of 10% glycerol to use as the competent cells.

Next, pKD46 was introduced into the competent cells of MG1655 Ptac-KKDyI strain by electroporation. The electroporation was carried out under the condition of an electric field intensity of 18 kV/cm, a condenser volume of 25 μF, and a resistance value of 200Ω using GENE PULSER II (supplied from BioRad). Subsequently, 1 mL of SOC medium (20 g/L of bacto tryptone, 5 g/L of yeast extract, 0.5 g/L of NaCl, 10 g/L of glucose) was added to the microbial cells having pKD46 introduced by the electroporation, the cells were cultured with shaking at 30° C. for 2 hours, and then applied onto LB agar medium containing 100 mg/L of ampicillin. After culturing at 30° C. overnight, a grown colony was refined on the same agar medium to obtain a strain MG1655 Ptac-KKDI/pKD46.

Competent cells of the obtained strain MG1655 Ptac-KKDI/pKD46 for the electroporation were prepared as follows. Cells of the strain MG1655 Ptac-KKDI/pKD46 were cultured with shaking in 5 mL of LB medium containing 100 mg/L of ampicillin at 30° C. overnight. Subsequently, 50 μL of the resulting cultured medium was inoculated to 5 mL of LB medium containing 100 mg/L of ampicillin, and the cells were cultured with shaking at 30° C. until absorbance at OD600 became around 0.6. Subsequently the microbial cells were collected, washed three times with ice-cooled 10% glycerol, and then finally suspended in 0.3 mL of 10% glycerol to use as the competent cells.

Next, a gene fragment for substituting a promoter region of the ppa gene on the chromosome was prepared. A nucleotide sequence of the ppa gene and its promoter region are available from existing database (NCBI Reference Sequences NC_000913.2, ppa gene locus tag: b4225, Range: 4447145 . . . 4447675, complement). Substitution of the promoter region of the ppa gene was carried out by a λ-red method. A genomic fragment having λattL-Tet-λattR-Ptac was used as a template for PCR. This includes a tac promoter (Ptac), a tetracycline resistant drug marker (Tet) and λattL and λattR that are attachment sites of λ phage. These nucleotide sequences are shown in SEQ ID NO:1. A PCR was carried out using primers consisting of the nucleotide sequences of SEQ ID NO:2 and SEQ ID NO:3. LA-Taq polymerase sold by TaKaRa Bio was utilized as DNA polymerase, and the reaction was carried out under a condition of 92° C. for one minute, 40 cycles (92° C. for 10 seconds, 54° C. for 20 seconds and 72° C. for 2 minutes) and 72° C. for 5 minutes. A gene fragment where sequences of upstream 60 bp and downstream 60 bp of the promoter region of the ppa gene had been added to each outer side of λattL-Tet-λattR-Ptac, respectively was amplified by PCR above. This gene fragment was purified using Wizard PCR Prep DNA Purification System (supplied from Promega). Hereinafter, the resulting gene fragment was designated as Tet-Ptac-ppa.

Next, Tet-Ptac-ppa was introduced into the competent cells of the strain MG1655 Ptac-KKDYI/pKD46 by the electroporation. The electroporation was carried out under the condition of the electric field intensity of 18 kV/cm, the condenser volume of 25 μF, and the resistance value of 200Ω using GENE PULSER II (supplied from BioRad). Subsequently, 1 mL of SOC medium was added to the competent cells, which were then cultured with shaking at 30° C. for 2 hours, and then applied onto LB agar medium containing 25 mg/L of tetracycline. After culturing at 37° C. overnight, a grown colony was refined using the same agar medium. Subsequently, colony PCR was carried out using primers consisting of the nucleotide sequences of SEQ ID NO:4 and SEQ ID NO:5 to confirm that the promoter region of the ppa gene was substituted with the tac promoter. The strain where the promoter region of the ppa gene had been substituted with the tac promoter was designated as a strain MG1655 Ptac-KKDyI Ptac-ppa.

### Example 2

### Analysis of PPA Expression in MG1655 Ptac-KKDyI Ptac-Ppa Strain

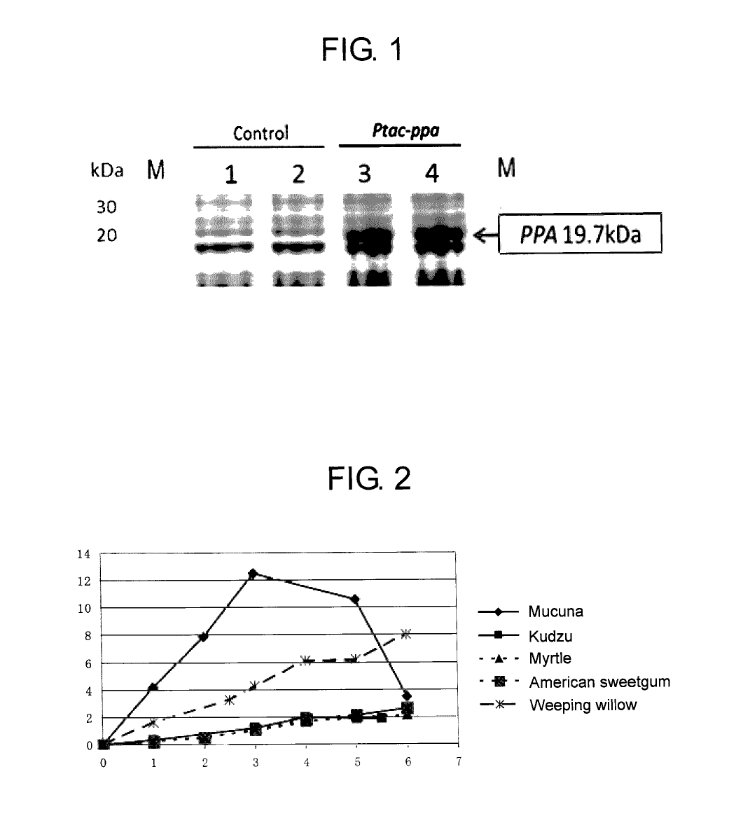

The expression amount of a protein of the pyrophosphate phosphatase (PPA) in MG1655 Ptac-KKDyI Ptac-ppa strain was confirmed by SDS-PAGE. Cells of MG1655 Ptac-KKDyI strain and MG1655 Ptac-KKDyI Ptac-ppa strain were cultured with shaking in 5 mL of LB medium at 37° C. overnight. The microbial cells after being collected were washed three times with ice-cooled 50 mM Tris buffer (Tris-HCl, pH 8.0), and disrupted using a sonicator (Bio-ruptor: ON for 30 seconds and OFF for 30 seconds for 20 minutes). The disrupted cell solution was centrifuged at 15,000 rpm for 10 minutes to remove cell debris. The resulting supernatant fraction was used as a soluble protein fraction. The soluble protein fraction was quantified by Bradford method, and 5 μg of the soluble protein was electrophoresed on SDS-PAGE (NuPAGE: SDS-PAGE Gel System supplied from Invitrogen). Subsequently, CBB staining and decoloration were carried out according to standard methods. A photograph of a gel showing bands around a PPA protein mass was shown in FIG. 1. As a result, increase of the expression amount of a protein presumed to be PPA was confirmed in MG1655 Ptac-KKDyI Ptac-ppa strain (FIG. 1). The expression amount of the protein presumed to be PPA in MG1655 Ptac-KKDyI Ptac-ppa strain was estimated to be about 2 to 5 folds larger than that of the original bacterial strain (control) from density of the bands on SDS-PAGE after the electrophoresis.

### Example 3

### Construction of MG1655 Ptac-KKDyI Ptac-Ppa/pSTV28-Ptac-ispSK/pMW-Para-mvaES Strain

Competent cells of MG1655 Ptac-KKDyI Ptac-ppa strain for electroporation were prepared as follows. Cells of MG1655 Ptac-KKDyI Ptac-ppa strain were cultured with shaking in 5 mL of LB medium at 37° C. overnight. Subsequently, 50 μL of the resulting cultured medium was inoculated to new 5 mL LB medium and cultured with shaking at 37° C. until absorbance at OD600 became around 0.6. Then, the microbial cells were collected, washed three times with ice-cooled 10% glycerol, and finally suspended in 0.5 mL of 10% glycerol to use as the competent cells.

An isoprene synthase-expressing plasmid derived from kudzu, pSTV28-Ptac-ispSK (see Reference Example 3-5) was introduced into the competent cells of MG1655 Ptac-KKDyI Ptac-ppa strain by the electroporation under the above condition. Subsequently, 1 mL of SOC medium was added to the competent cells, which were then cultured at 30° C. for 2 hours, and then applied onto LB agar medium containing 60 mg/mL of chloramphenicol. After culturing at 37° C. overnight, a grown colony was refined in the same agar medium to obtain MG1655 Ptac-KKDyI Ptac-ppa/pSTV28-Ptac-ispSK strain having introduced pSTV28-Ptac-ispSK.

Subsequently, pMW-Para-mvaES-Ttrp (see Reference Example 7-3) was introduced into MG1655 Ptac-KKDyI Ptac-ppa/pSTV28-Ptac-ispSK strain. As with above, competent cells of MG1655 Ptac-KKDyI Ptac-ppa/pSTV28-Ptac-ispSK strain were prepared, and then pMW-Para-mvaES-Ttrp was introduced by the electroporation under the above condition. Subsequently, 1 mL of SOC medium was added to the competent cells, which were then cultured with shaking at 30° C. for 2 hours, and then applied onto LB agar medium containing 60 mg/mL of chloramphenicol and 100 mg/L of kanamycin. After culturing at 37° C. overnight, a grown colony was refined in the same agar medium to obtain MG1655 Ptac-KKDyI Ptac-ppa/pSTV28-Ptac-ispSK/pMW-Para-mvaES-Ttrp strain having introduced pMW-Para-mvaES-Ttrp. Hereinafter, MG1655 Ptac-KKDyI Ptac-ppa/pSTV28-Ptac-ispSK/pMW-Para-mvaES-Ttrp strain where the expression of the ppa gene was enhanced is described as a ppa expression-enhanced strain.

### Example 4

### Evaluation of Jar Cultivation of Ppa Expression-Enhanced Strain

Jar cultivation of the ppa expression-enhanced strain and a control strain (MG1655 Ptac-KKDyI/pSTV28-Ptac-ispSK/pMW-Para-mvaES-Ttrp) was evaluated. Cells were applied onto the LB agar medium containing 60 mg/mL of chloramphenicol and 100 mg/L of kanamycin, and cultured at 34° C. for 16 hours. Subsequently, 0.3 L of glucose medium described in Table 1 was placed in a 1 L volume fermenter, and the microbial cells sufficiently grown on one plate were inoculated thereto and cultured. A culture condition was pH 7.0 (controlled with ammonia gas), 30° C., ventilation of 150 mL/minute, and stirring such that an oxygen concentration in the medium was 5% or higher. After absorbance at OD600 reached around 20, L-arabinose at final concentration of 20 mM was added to the medium, and the cultivation was carried out for 45 hours. During the cultivation, a glucose solution prepared at 500 g/L was appropriately added such that a glucose concentration in the medium was kept at 10 g/L or higher. Evolved gas was collected in a 1 L gas bag with time, and a concentration of isoprene gas contained in the evolved gas was measured. An analysis condition for the isoprene gas was described below. An analysis condition for gas chromatography is the same as described in Reference Example 4-3.

TABLE 1
Composition of glucose medium

| | Final concentration | |
|---|---|---|
| Group A | | |
| Glucose | 80 | g/L |
| MgSO₄•7aq | 2.0 | g/L |
| Group B | | |
| (NH₄)₂SO₄ | 2.0 | g/L |
| KH₂PO₄ | 2.0 | g/L |
| FeSO₄•7aq | 20 | mg/L |
| MnSO₄•5aq | 20 | mg/L |
| Yeast Extract | 4.0 | g/L |

After preparing 0.15 L of Group A and 0.15 L of Group B, they were heated and sterilized at 115° C. for 10 minutes. After cooling, Group A and Group B were mixed, and 60 mg/mL of chloramphenicol and 100 mg/L of kanamycin were added thereto to use as the medium.

The amount of isoprene per jar (mg/B) and a glucose consumption rate (%) after the cultivation for 45 hours in the control strain and the ppa expression-enhanced strain are described in Table 2. Both the amount of isoprene produced per jar (mg/B) and the glucose consumption rate (%) could be confirmed to be higher in the ppa expression-enhanced strain than in the control strain.

TABLE 2

| | Amount of isoprene produced | Glucose consumption |
|---|---|---|
| Strain name | per jar (mg/B) | rate (%) |
| Control strain | 436 | 2.87 |
| ppa Expression- | 657 | 4.59 |
| enhanced strain | | |

### Reference Example 1

### Evaluation of Ability to Produce Isoprene in Plants

### 1-1) Measurement of Amount of Isoprene Formed Per Unit Weight of Dry Leaves

First, an amount of isoprene formed per 1 g of dry leaves in the plant was measured for evaluating an ability to produce isoprene in plants. Mucuna (Mucuna bracteata), Weeping willow (Salix babylonica), American sweetgum (Liquidambar styraciflua), Myrtle (Myrtus communis), and Kudzu (Pueraria lobata) were used as the plants.

In the measurement of an amount of formed isoprene, a gas replaceable desiccator (trade name: Vacuum Desiccator, manufactured by AS ONE Corporation) was housed in an incubator (trade name: Growth Chamber MLR-351H, manufactured by SANYO), and the incubator was set to a high temperature induction condition (an illuminance of 100 μmol E/m²/s at 40° C.) while a fan for stirring the gas, which was provided in the gas replaceable desiccator, was driven to stir an atmosphere in space in the gas replaceable desiccator. After the temperature of the atmosphere in the gas replaceable desiccator reached 40° C., a plant body of Mucuna planted in a planter was housed therein and kept for 3 hours in a state where the gas replaceable desiccator was sealed. Then, a gas component released from Mucuna was aspirated from the space in the gas replaceable desiccator by an aspiration pump through a silicon tube, an adsorption tube and a gas collection tube. Thereby, water vapor (water content) contained in the gas component released from Mucuna was adsorbed and separated in the adsorption tube, the gas component from which the water vapor had been separated was led to the gas collection tube, and the gas component was collected in the gas collection tube. Subsequently, isoprene contained in the gas component collected in the gas collection tube was quantitatively analyzed using gas chromatograph (trade name: GC-FID6890, manufactured by Agilent).

For the weight of dry leaves, a leaf area of a fresh individual leaf, and a dry weight when the fresh individual leaf is dried by a dryer at 80° C. for 8 hours establish a very good positive correlation. Thus, a formula for converting from the leaf area to the dry weight was derived, and the dry weight was estimated from the entire leaf area from the plant body of Mucuna used for the measurement of an amount of formed isoprene.

The amount of formed isoprene per 1 g of the dry leaf was obtained by dividing the amount of formed isoprene from the entire plant body of Mucuna by the estimated weight of the entire plant body.

As a result, it was demonstrated that Mucuna was excellent in amount of formed isoprene per unit weight of the dry leaf (FIG. 2).

### 1-2) Measurement of Amount of Formed Isoprene Per Amount of Total Protein

Then, the amount of formed isoprene per amount of total protein extracted from leaves of various plants was measured. Mucuna (samples 1 and 2), Weeping willow, American sweetgum, Myrtle, and Kudzu were used as the plants.

For extraction of the protein, a buffer solution (50 mM Tris-HCl, 20 mM MgCl, 5% glycerol, 0.02% Triton-X100, pH 8.0) was made, and 10% Polyclar AT, 20 mM DTT, protease complete tablet (one tablet/50 mL), and 1 mM benzamidine HCl (final concentrations, each) were added just before the use, and was used as a protein extraction buffer. 50 mL of the protein extraction buffer was added to 5 g of the sample, then the mixture was ground well in a cold mortar on ice and filtrated though doubly overlapped Miracloth. A filtrate was centrifuged at 12,000 G for 20 minutes and 40,000 G for 40 minutes to obtain a supernatant, and the supernatant was used as a crude extract.

Subsequently, this crude extract was fractionated with ammonium sulfate. Proteins precipitated in a range of 40% to 55% of final concentrations of ammonium sulfate were centrifuged at 40,000 G for 40 minutes, and an obtained pellet was re-dissolved in the protein extraction buffer to obtain an ammonium sulfate fraction.

A total (ammonium sulfate fraction) protein mass was calculated by measuring the ammonium sulfate fraction using Bradford assay. A Bradford reagent was reacted with the standard protein, bovine serum albumin, and absorbance at a wavelength of 595 nm was measured using a spectrophotometer. A standard curve for the protein was made using the obtained absorbance values. The absorbance at a wavelength of 595 nm was also measured in the ammonium sulfate fraction diluted to 50 times, and the amount of the total (ammonium sulfate fraction) protein was estimated from the standard curve for the standard protein.

In the measurement of the amount of formed isoprene, 100 μL of the crude extract or 100 μL of a crude enzyme solution boiled at 100° C. was placed in a 4 mL glass vial, and then 2 μL of a 0.5 M MgCl₂ solution and 5 μL of a 0.2 M DMAPP solution were added thereto. The vial was tightly closed with a screw cap with a septum, and then the vial was gently vortexed and set in an incubator at 40° C. After 0.5, 1 and 2 hours, 0.5 to 2 mL of a gas layer in a headspace was sampled by a gas-tight syringe, and the amount of formed isoprene was measured using gas chromatograph (trade name: GC-FID6890, manufactured by Agilent). The amount of formed isoprene using the crude enzyme after 0.5, 1 and 2 hours was calculated by subtracting a measured value in the case of using the crude enzyme solution boiled at 100° C. from a measured value in the case of using the crude enzyme. An enzymatic activity per 1 mg of the total protein (specific activity) was calculated from the amount of the formed isoprene per one hour. The amount of formed isoprene was measured with keeping the amount of DMAPP that was the substrate of the isoprene synthase constant.

As a result, it was demonstrated that Mucuna was excellent in amount of formed isoprene per amount of total protein (FIG. 3, Table 3). As described above, it was shown that Mucuna was excellent in ability to produce isoprene.

TABLE 3
Amount of formed isoprene per amount of total protein (index numbers relative to case of Kudzu)

| | | | | | Specific activity index (Value from |
|---|---|---|---|---|---|
| | 0 hour* | 0.5 hour* | 1 hour* | 2 hours* | Kudzu was set to 1) |
| Mucuna 1 | 0 | 16.947 | 61.895 | 160.632 | 16.87842808 |
| Mucuna 2 | 0 | 0 | 183.587 | 449.514 | 47.23274141 |
| American sweetgum | 0 | 0 | 22.063 | 46.132 | 4.847325838 |
| Weeping willow | 0 | 0 | 9.756 | 24.39 | 2.562782389 |
| Myrtle | 0 | 0 | 0 | 27.451 | 2.884417358 |
| Kudzu | 0 | 0 | 6.662 | 9.517 | 1 |

*Unit is μg isoprene/mg protein

### Reference Example 2

### Cloning of Isoprene Synthase Gene Derived from Mucuna

### 2-1) Evaluation of Sampling Time

Isoprene gas released from leaves of Mucuna illuminated with light for 1, 2, 3, and 5 hours at temperature of 40° C. was sampled and the amount of produced isoprene was quantified by gas chromatography described later, and production of 4, 8, 12, and 10 μg of isoprene/g DW leaf was confirmed. Thus, it was confirmed that an optimal light illumination time was 3 hours.

### 2-2) Extraction of Total RNA Lysis Solution

A total RNA was extracted from leaves of Mucuna with total RNA lysis solution according to the following procedures.

(1) The leaves of Mucuna illuminated with light for 3 hours at temperature of 40° C. were sampled.

(2) 100 mg of leaf tissue was pulverized in a mortar with rapidly freezing the leaf tissue with liquid nitrogen, then the leaf tissue together with the liquid nitrogen was dispensed in an RNA-free 2 mL Eppendorf tube, and the liquid nitrogen was gasified.

(3) To this Eppendorf tube, 450 μL of a dissolution buffer RLT (containing 2-mercaptoethanol) attached to RNeasy Plant Kit (manufactured by Qiagen), and mixed vigorously with Vortex to obtain a leaf tissue lysate.

(4) This leaf tissue lysate was applied to QIAshredder spin column attached to RNeasy Plant Kit, and centrifuged at 15,000 rpm for 2 minutes.

(5) A supernatant alone of a column eluate was transferred to a new RNA-free 2 mL Eppendorf tube, then special grade ethanol in a half volume of the supernatant was added to the supernatant, and the obtained solution was mixed by pipetting to obtain about 650 μL of a solution.

(6) This solution was applied to RNeasy spin column attached to RNeasy Plant Kit, centrifuged at 10,000 rpm for 15 seconds, and a filtrate was discarded.

(7) 700 μL of RW1 buffer attached to RNeasy Plant Kit was added to this RNeasy spin column, centrifuged at 10,000 rpm for 15 seconds, and a filtrate was discarded.

(8) 500 μL of BPE buffer attached to RNeasy Plant Kit was added to this RNeasy spin column, centrifuged at 10,000 rpm for 15 seconds, and a filtrate was discarded.

(9) 500 μL of BPE buffer was again added to this RNeasy spin column, centrifuged at 10,000 rpm for 2 minutes, and a filtrate was discarded.

(10) This RNeasy spin column was set to a 2 mL collective tube attached to RNeasy Plant Kit, centrifuged at 15,000 rpm for one minute, and a filtrate was discarded.

(11) This RNeasy spin column was set to a 1.5 mL collective tube attached to RNeasy Plant Kit.

(12) RNA-free distilled water attached to RNeasy Plant Kit was directly added to a membrane of this RNeasy spin column using a Pipetman, centrifuged at 10,000 rpm for one minute, and total RNA was collected. This step was repeated twice to obtain about 100 μg of total RNA.

2-3) Analysis of Nucleotide Sequence of Isoprene Synthase Gene Derived from Mucuna

Quality of RNA in the extracted total RNA solution was checked using nano-chips for RNA provided by BioAnalyzer (Agilent Technologies, Inc.), and it was confirmed that the solution was not contaminated with genomic DNA and RNA was not decomposed in the solution.

This total RNA was converted into a double strand using reverse transcriptase, and then fragmented using a nebulizer. Nucleotide sequences of 198,179 fragments having a poly A sequence at a 3′ end were analyzed using 454 titanium FLX high performance sequencer (manufactured by Roche Applied Science). Overlapped sequences in the obtained fragment sequences were aligned to obtain 13,485 contig sequences. BLAST search was performed for these contig sequences, and 6 contig sequences having the homology (identity of nucleotide sequences) to registered and known isoprene synthase gene sequences from Kudzu and Poplar were extracted. These sequences were further analyzed in detail, and 3 sequences in these 6 contig sequences were found to be derived from the same gene. Thus, a partial sequence of the isoprene synthase gene derived from Mucuna was obtained. 5′ RACE was performed based on this partial sequence to obtain a full length nucleotide sequence of the isoprene synthase cDNA derived from Mucuna, which was represented by SEQ ID NO:6.

### Reference Example 3

### Preparation of Expression Plasmid for Isoprene Synthase Derived from various plants

3-1) Chemical Synthesis of Isoprene Synthase Derived from Pueraria montana Var. Lobata (Kudzu)

The nucleotide sequence and the amino acid sequence of the isoprene synthase cDNA derived from Pueraria montana var. lobata were already known (ACCESSION: AAQ84170: P. montana var. lobata isoprene synthase (IspS)). The amino acid sequence of the IspS protein derived from P. montana and the nucleotide sequence of its cDNA are represented by SEQ ID NO:8 and SEQ ID NO:9, respectively. The IspS gene was optimized for codon usage frequency in E. coli in order to efficiently express the IspS gene in E. coli, and further designed to cut off the chloroplast localization signal. The designed gene was designated as IspSK. A nucleotide sequence of IspSK is represented by SEQ ID NO:10. The IspSK gene was chemically synthesized, then cloned into pUC57 (manufactured by GenScript), and the resulting plasmid was designated as pUC5-IspSK.

3-2) Chemical Synthesis of Isoprene Synthase Derived from Populus alba x Populus Tremula (Poplar)

The nucleotide sequence of the isoprene synthase cDNA and the amino acid sequence of the isoprene synthase derived from P. alba x P. tremula were already known (ACCESSION: CAC35696: P. alba x P. tremula (Poplar) isoprene synthase). The amino acid sequence of the IspS protein derived from P. alba x P. tremula and the nucleotide sequence of its cDNA are represented by SEQ ID NO:11 and SEQ ID NO:12, respectively. An IspS gene that was optimized for the codon usage frequency in E. coli in the same manner as above and in which the chloroplast localization signal was cut off was designed and designated as IspSP. A nucleotide sequence of IspSP is represented by SEQ ID NO:13. The IspSP gene was chemically synthesized, then cloned into pUC57 (manufactured by GenScript), and the resulting plasmid was designated as pUC57-IspSP.

3-3) Chemical Synthesis of Isoprene Synthase Derived from Mucuna bracteata (Mucuna) Based on the nucleotide sequence of the isoprene synthase cDNA derived from Mucuna bracteata, an IspS gene that was optimized for the codon usage frequency in E. coli was designed in the same manner as above. One in which the chloroplast localization signal had been conferred was designated as IspSM (L), and one in which the chloroplast localization signal had been cut off was designated as IspSM. Nucleotide sequences for IspSM (L) and IspSM are represented by SEQ ID NO:14 and SEQ ID NO:15, respectively. The IspSM gene and the IspSM (L) gene were chemically synthesized, then cloned into pUC57 (manufactured by GenScript), and the resulting plasmids were designated as pUC57-IspSM and pUC57-IspSM (L).
3-4) Construction of Expression Plasmid, pSTV28-Ptac-Ttrp

An expression plasmid pSTV28-Ptac-Ttrp for expressing IspS derived from various plants in E. coli was constructed. First, a DNA fragment comprising a tac promoter (synonym: Ptac) region (deBoer, et al., (1983) Proc. Natl. Acad. Sci. U.S.A., 80, 21-25, which is incorporated herein by reference in its entirety) and a terminator region of tryptophan operon (synonym: Ttrp) derived from E. coli (Wu et al., (1978) Proc. Natl. Acad. Sci. U.S.A., 75, 442-5446, which is incorporated herein by reference in its entirety) and having a KpnI site at a 5′ terminus and a BamHI site at a 3′ end was synthesized chemically (the nucleotide sequence of Ptac-Ttrp is represented by SEQ ID NO:16). The resulting Ptac-Ttrp DNA fragment was digested with KpnI and BamHI, and ligated to pSTV28 (manufactured by Takara Bio Inc.) similarly digested with KpnI and BarnHI by a ligation reaction with DNA ligase. The resulting plasmid was designated as pSTV28-Ptac-Ttrp (its nucleotide sequence is represented by SEQ ID NO:17). This plasmid can amplify the expression of the IspS gene by cloning the IspS gene downstream of Ptac.

3-5) Construction of Plasmid for Expressing IspS Gene Derived from Various Plants

Plasmids for expressing the IspSK gene, the IspSP gene, the IspSM gene and the IspSM (L) gene in E. coli were constructed by the following procedure. PCR was performed with Prime Star polymerase (manufactured by Takara Bio Inc.) using synthesized oligonucleotides consisting of the nucleotide sequences of SEQ ID NOs:18 and 19 as primers with pUC57-IspSK as a template, synthesized oligonucleotides consisting of the nucleotide sequences of SEQ ID NOs:20 and 21 as primers with pUC57-IspSP as a template, synthesized oligonucleotides consisting of the nucleotide sequences of SEQ ID NOs:22 and 23 as primers with pUC57-IspSM as a template, or further synthesized oligonucleotides consisting of the nucleotide sequences of SEQ ID NOs:24 and 25 as primers with pUC57-IspSM (L) as a template. A reaction solution was prepared according to a composition attached to the kit, and a reaction at 98° C. for 10 seconds, 54° C. for 20 seconds and 68° C. for 120 seconds was performed in 40 cycles. As a result, a PCR product containing the IspSK gene, the IspSP gene, the IspSM gene or the IspSM (L) gene was obtained. Likewise, PCR was performed with Prime Star polymerase (manufactured by Takara Bio Inc.) using synthesized oligonucleotides consisting of the nucleotide sequences of SEQ ID NOs:26 and 27 as primers with pSTV28-Ptac-Ttrp as a template. A reaction solution was prepared according to a composition attached to the kit, and a reaction at 98° C. for 10 seconds, 54° C. for 20 seconds and 68° C. for 210 seconds was performed in 40 cycles. As a result, a PCR product containing pSTV28-Ptac-Ttrp was obtained. Subsequently, the purified IspSK gene, IspSP gene, IspSM gene, and IspSM (L) gene fragments were ligated to the PCR product for pSTV28-Ptac-Ttrp using In-Fusion HD Cloning Kit (manufactured by Clontech). The resulting plasmids for expressing the IspSK gene, the IspSP gene, IspSM gene and IspSM (L) gene were designated as pSTV28-Ptac-IspSK, pSTV28-Ptac-IspSP, pSTV28-Ptac-IspSM, and pSTV28-Ptac-IspSM (L), respectively.

TABLE 4
Primer sequences used for construction
of plasmids for expressing IspS genes
derived from various plants

| Subject for | Sequence | |
|---|---|---|
| amplification | name | Sequence (5′-) |
| IspSK | Ptac- | GATAACAATTTCACA |
| | IspS(K)F | CAATAATTTTGTTTA |
| | | ACTTTAAGAAGGAGA |
| | | TATAATGTGTGCGAC |
| | | CTCTTCTCAATTTAC |
| | | TCAG |
| | | (SEQ ID NO: 18) |
| IspSK | IspS(K)R- | ACGGCCAGTGAATTC |
| | MCSR | TTAGACATACATCAG |
| | | CTGGTTAATCGG |
| | | (SEQ ID NO: 19) |
| IspSP | Ptac- | GATAACAATTTCACA |
| | IspS(P)F | CAATAATTTTGTTTA |
| | | ACTTTAAGAAGGAGA |
| | | TATAATGTGCTCTGT |
| | | TTCTACCGAGAACGT |
| | | TTCC |
| | | (SEQ ID NO: 20) |
| IspSP | IspS(P)R- | ACGGCCAGTGAATTC |
| | MCSR | TTAACGTTCGAACGG |
| | | CAGAATCGGTTCG |
| | | (SEQ ID NO: 21) |
| IspSM | Ptac- | GATAACAATTTCACA |
| | IspS(M)F | CAATAATTTTGTTTA |
| | | ACTTTAAGAAGGAGA |
| | | TATAATGTCCGCCGT |
| | | TTCAAGCCA |
| | | (SEQ ID NO: 22) |
| IspSM | IspS(M)R- | ACGGCCAGTGAATTC |
| | MCSR | TTAGTTAATCGGGAA |
| | | CGGGT |
| | | (SEQ ID NO: 23) |
| IspSM(L) | Ptac- | GATAACAATTTCACA |
| | IspS(M(L)) | CAATAATTTTGTTTA |
| | F | ACTTTAAGAAGGAGA |
| | | TATAATGGCTACCAA |
| | | CCCGTCCTGTCTGTC |
| | | AACC |
| | | (SEQ ID NO: 24) |
| IspSM(L) | IspS(M(L)) | ACGGCCAGTGAATTC |
| | R-MCSR | TCAGTTAATCGGGAA |
| | | CGGGT |
| | | (SEQ ID NO: 25) |
| pSTV28-Ptac- | pSTV28-F | GTGTGAAATTGTTAT |
| Ttrp | | CCGCTCACAATTCC |
| | | (SEQ ID NO: 26) |
| pSTV28-Ptac- | pSTV28-R | GAATTCACTGGCCGT |
| Ttrp | | CGTTTTACAACG |
| | | (SEQ ID NO: 27) |

### Reference Example 4

### Measurement of Enzymatic Activity of Isoprene Synthase Derived from Various Plants Using Crude Enzyme Extract Derived from E. coli

4-1) Construction of E. coli MG1655 Strain Having Ability to Produce Isoprene

Competent cells of E. coli MG1655 strain (ATCC 700926) were prepared, and then pSTV28-Ptac-Ttrp, pSTV28-Ptac-IspSK, pSTV28-Ptac-IspSP, pSTV28-Ptac-IspSM, or further pSTV28-Ptac-IspSM (L) was introduced therein by an electroporation method. A suspension of the cells was evenly applied onto an LB plate containing 60 mg/L of chloramphenicol, and cultured at 37° C. for 18 hours. Subsequently, transformants that were resistant to chloramphenicol were obtained from the resulting plate. A strain in which pSTV28-Ptac-Ttrp, pSTV28-Ptac-IspSK, pSTV28-Ptac-IspSP, pSTV28-Ptac-IspSM, or further pSTV28-Ptac-IspSM (L) was introduced into E. coli MG1655 strain were designated as MG1655/pSTV28-Ptac-Ttrp, MG1655/pSTV28-Ptac-IspSK, MG1655/pSTV28-Ptac-IspSP, MG1655/pSTV28-Ptac-IspSM, or further MG1655/pSTV28-Ptac-IspSM (L) strain, respectively.

### 4-2) Method of Preparing Crude Enzyme Extract

Microbial cells of MG1655/pSTV28-Ptac-Ttrp, MG1655/pSTV28-Ptac-IspSK, MG1655/pSTV28-Ptac-IspSP, MG1655/pSTV28-Ptac-IspSM, or MG1655/pSTV28-Ptac-IspSM (L) strain were evenly applied onto the LB plate containing 60 mg/L of chloramphenicol, and cultured at 37° C. for 18 hours. The microbial cells corresponding to ⅙ of the resulting plate were inoculated to a Sakaguchi flask in which 20 mL of LB containing 60 mg/L of chloramphenicol had been added, and cultured at 37° C. for 6 hours. The microbial cells from the culture medium were centrifuged at 5000 rpm at 4° C. for 5 minutes, and washed twice with ice-cold isoprene synthase buffer (50 mM Tris-HCl, pH 8.0, 20 mM MgCl₂, 5% glycerol). The washed microbial cells were suspended in 1.8 mL of the same buffer. About 0.9 mL of beads for disruption (YBG01, diameter 0.1 mm) and 0.9 mL of the microbial cell suspension were placed in a 2 mL tube specific for a multibead shocker, and the microbial cells were disrupted using the multibead shocker manufactured by Yasui Kikai Corporation at 2500 rpm at 4° C. for 3 cycles of ON for 30 seconds/OFF for 30 seconds. After the disruption, the tube was centrifuged at 20,000 g at 4° C. for 20 minutes, and a supernatant was used as a crude enzyme extract.

### 4-3) Measurement of Isoprene Synthase Activity

The crude enzyme extract from MG1655/pSTV28-Ptac-Ttrp, MG1655/pSTV28-Ptac-IspSK, MG1655/pSTV28-Ptac-IspSP, MG1655/pSTV28-Ptac-IspSM, or MG1655/pSTV28-Ptac-IspSM (L) strain (containing 2 mg as amount of total protein) together with the isoprene buffer in a total volume of 0.5 mL was placed in a headspace vial (22 mL CLEAR CRIMP TOP VIAL (cat #B0104236) manufactured by Perkin Elmer), then 0.025 mL of a 0.5 M MgCl₂ solution and 0.01 mL of a 0.2 M DMAPP (manufactured by Cayman, catalog No. 63180) solution were added thereto, and the mixture was lightly vortexed. Then immediately, the vial was tightly sealed with a cap with a butyl rubber septum for the headspace vial (CRIMPS (cat #B0104240) manufactured by Perkin Elmer), and kept at 37° C. for 2 hours.

After completion of the reaction, a concentration of isoprene in the headspace of the vial was measured by gas chromatography. An analysis condition for the gas chromatography will be described below.

Headspace sampler (manufactured by Perkin Elmer, Turbo Matrix 40)
Temperature for keeping vial warm: 40° C.
Time period for keeping vial warm: 30 minutes
Pressurization time: 3.0 minutes
Injection time: 0.02 minute
Needle temperature: 70° C.
Transfer temperature: 80° C.
Carrier gas pressure (high purity helium): 124 kPa
Gas chromatography (manufactured by Shimadzu Corporation, GC-2010 Plus AF)
Column (Rxi (registered trademark)-1 ms: length 30 m, internal diameter 0.53 mm, liquid
phase film thickness 1.5 μm, cat #13370)
Column temperature: 37° C.

### Pressure: 24.8 kPa

Column flow: 5 mL/minute
Influx method: Split 1:0 (actually measured 1:18)
Transfer flow: 90 mL
GC injection volume: 1.8 mL (transfer flow×injection time)
Injection volume of sample into column: 0.1 mL
Inlet temperature: 250° C.
Detector: FID (hydrogen 40 mL/minute, air 400 mL/minute, makeup gas helium 30 mL/minute)
Detector temperature: 250° C.

### Preparation of Isoprene Standard Sample

A reagent isoprene (specific gravity 0.681) was diluted to 10, 100, 1000, 10000, and 100000 times with cold methanol to prepare standard solutions for addition. Subsequently, 1 μL of each standard solution for addition was added to a headspace vial in which 1 mL of water had been added, and used as a standard sample.

The amount of formed isoprene after the reaction of each microbial strain for 2 hours is described in Table 5.

TABLE 5
Amount of formed isoprene after reaction for 2 hours

| Name of microbial strain | Amount of formed isoprene (mg/L) |
|---|---|
| MG1655/pSTV28-Ptac-Ttrp | 0.10 ± 0.01 |
| MG1655/pSTV28-Ptac-IspSK | 0.45 ± 0.02 |
| MG1655/pSTV28-Ptac-IspSM | 28.93 ± 6.04 |
| MG1655/pSTV28-Ptac-IspSM (L) | 5.06 ± 0.13 |
| MG1655/pSTV28-Ptac-IspSP | 0.10 ± 0.01 |

From the result in Table 5, the amount of formed isoprene was larger in order of MG1655/pSTV28-Ptac-IspSM, MG1655/pSTV28-Ptac-IspSM (L) and MG1655/pSTV28-Ptac-IspSK strains, and was almost equal in MG1655/pSTV28-Ptac-IspSP and MG1655/pSTV28-Ptac-Ttrp strains. From the above result, the crude enzyme extract from the strain introduced with the isoprene synthase derived from Mucuna exhibited the highest activity to form isoprene.

### Reference Example 5

### Effects of Introduction of Isoprene Synthase Derived from Various Plants on E. coli MG1655 Strain

From the result of the crude enzymatic activity in Reference Example 4, the highest activity was confirmed in the isoprene synthase derived from Mucuna that deleted the chloroplast localization signal. Thus, an ability to produce isoprene from glucose was compared in all isoprene synthase-introduced strains in which the chloroplast localization signal had been deleted. Microbial cells of MG1655/pSTV28-Ptac-Ttrp, MG1655/pSTV28-Ptac-IspSK, MG1655/pSTV28-Ptac-IspSP, or MG1655/pSTV28-Ptac-IspSM strain were evenly applied onto the LB plate containing 60 mg/L of chloramphenicol, and cultured at 37° C. for 18 hours. One loopful of the microbial cells from the resulting plate was inoculated to 1 mL of M9 glucose medium in a headspace vial. The vial was tightly sealed with the cap with the butyl rubber septum for the headspace vial (CRIMPS (cat #B0104240) manufactured by Perkin Elmer), and the microbial cells were cultured at 30° C. for 24 hours using a reciprocal shaking cultivation apparatus (120 rpm). A composition of the M9 glucose medium is as described in Table 6.

TABLE 6
Composition of M9 glucose medium

| | |
|---|---|
| Glucose | 1.0 g/L |
| Na₂HPO₄ | 6.0 g/L |
| KH₂PO₄ | 3.0 g/L |
| NaCl | 0.5 g/L |
| NH₄Cl | 1.0 g/L |
| 1M MgSO₄ (autoclaved) | 1.0 mL |
| 1M CaCl₂ (autoclaved) | 0.1 mL |

Further, chloramphenicol was added at a final concentration of 60 mg/L. The volume was adjusted to 1 L and the medium was then sterilized by filtration.

After completion of the cultivation, the concentration of isoprene in the headspace in the vial was measured by the gas chromatography. An OD value was also measured at 600 nm using a spectrophotometer (HITACHI U-2900). The concentration of isoprene and the OD value in each microbial strain at the time of completing the cultivation are described in Table 7.

TABLE 7
OD value, and amount (μg/L) of isoprene produced by
MG1655/pSTV28-Ptac-Ttrp, MG1655/pSTV28-Ptac-IspSK,
MG1655/pSTV28-Ptac-IspSP and MG1655/pSTV28-Ptac-IspSM
strains at the time of completing cultivation

| | | Amount (μg/L) of formed |
|---|---|---|
| Name of microbial strain | OD value | isoprene |
| MG1655/pSTV28-Ptac-Ttrp | 1.68 ± 0.04 | ND |
| MG1655/pSTV28-Ptac-IspSK | 1.60 ± 0.09 | 43 ± 6 |
| MG1655/pSTV28-Ptac-IspSM | 1.45 ± 0.03 | 56 ± 7 |
| MG1655/pSTV28-Ptac-IspSP | 1.59 ± 0.07 | 26 ± 3 |

From the results in Table 7, it was found that the amount of produced isoprene was larger in order of MG1655/pSTV28-Ptac-IspSM, MG1655/pSTV28-Ptac-IspSK, MG1655/pSTV28-Ptac-IspSP and MG1655/pSTV28-Ptac-Ttrp strains. From the above results, the strain introduced with the isoprene synthase derived from Mucuna exhibited the highest activity to produce isoprene in the wild strains.

### Reference Example 6

### Effects of Introduction of Isoprene Synthase Derived from Various Plants on E. coli MG1655 Strain in which MEP (Methylerythritol) Pathway is Enhanced

6-1) Construction of Plasmid for Expressing Dxs Gene (pMW219-Dxs)

It was already reported that the amount of formed isoprene was enhanced (Appl. Microbiol. Biotechnol., (2011) 90, 1915-1922, which is incorporated herein by reference in its entirety), when the expression of a dxs (1-deoxy-D-xylulose-5-phosphate synthase) gene that constitutes the MEP pathway was enhanced in E. coli strain in which the isoprene synthase was introduced. Thus, it was confirmed whether an ability to produce isoprene was also different due to an origin of the isoprene synthase in the strain in which the expression of the dxs gene was enhanced. The entire genomic nucleotide sequence of E. coli K-12 strain was already shown (GenBank Accession No. U00096) (Science, (1997) 277, 1453-1474, which is incorporated herein by reference in its entirety). pMW219 (manufactured by Nippon Gene Co., Ltd.) was used for amplifying the gene. This plasmid can increase an expression level of an objective gene when isopropyl-β-thiogalactopyranoside (IPTG) is added by introducing the objective gene into a multicloning site. Synthesized oligonucleotides were synthesized from the nucleotide sequences of SEQ ID NOs:28 and 29 based on the nucleotide sequence of the dxs gene in the genomic nucleotide sequence of E. coli. Subsequently, PCR was performed with Prime Star polymerase (manufactured by Takara Bio Inc.) using the synthesized oligonucleotides consisting of the nucleotide sequences of SEQ ID NOs:28 and 29 as the primers with MR1655 strain genomic DNA as the template. A reaction solution was prepared according to the composition attached to the kit, and a reaction at 98° C. for 10 seconds, 54° C. for 20 seconds and 68° C. for 120 seconds was performed in 40 cycles. As a result, a PCR product containing the dxs gene was obtained. Likewise, PCR was performed with Prime Star polymerase (manufactured by Takara Bio Inc.) using the synthesized oligonucleotides consisting of the nucleotide sequences of SEQ ID NOs:30 and 31 as the primers with pMW219 as the template. A reaction solution was prepared according to the composition attached to the kit, and a reaction at 98° C. for 10 seconds, 54° C. for 20 seconds and 68° C. for 240 seconds was performed in 40 cycles. As a result, a PCR product containing pMW219 was obtained. Subsequently, the purified dxs gene fragment was ligated to the PCR product of pMW219 using In-Fusion HD Cloning Kit (manufactured by Clontech). The resulting plasmid for expressing the dxs gene was designated as pMW219-dxs.

TABLE 8
Primer sequences used for construction
of plasmid for expressing dxs gene

| Sequence name | Sequence (5′-) |
|---|---|
| dxs-F | CAGGAAACAGCTATGAGTTTTGATA |
| | TTGCCAAATACCCGAC |
| | (SEQ ID NO: 28) |
| dxs-R | GCTGCCACTCCTGCTATACTCGTCA |
| | TAC (SEQ ID NO: 29) |
| pMW219-F | CATAGCTGTTTCCTGTGTGAAATTG |
| | TTATC (SEQ ID NO: 30) |
| pMW219-R | AGCAGGAGTGGCAGCGAATTCGAGC |
| | TCGGTACCCGGGGAT |
| | (SEQ ID NO: 31) |

6-2) Introduction of pMW219-Dxs into E. coli MG1655 Strain Having Ability to Produce Isoprene

Competent cells of MG1655/pSTV28-Ptac-Ttrp, MG1655/pSTV28-Ptac-IspSK, MG1655/pSTV28-Ptac-IspSM, or further MG1655/pSTV28-Ptac-IspSP strain were prepared, and pMW219-dxs was introduced therein by an electroporation method. The cells were evenly applied onto the LB plate containing 60 mg/L of chloramphenicol and 50 mg/L of kanamycin hydrochloride, and the cells were cultured at 37° C. for 18 hours. Transformants that were resistant to chloramphenicol and kanamycin were obtained from the resulting LB plates. Strains in which pMW219-dxs had been introduced into MG1655/pSTV28-Ptac-Ttrp, MG1655/pSTV28-Ptac-IspSK, MG1655/pSTV28-Ptac-IspSM, or further MG1655/pSTV28-Ptac-IspSP strain were designated as MG1655/pSTV28-Ptac-Ttrp/pMW219-dxs, MG1655/pSTV28-Ptac-IspSK/pMW219-dxs, MG1655/pSTV28-Ptac-IspSM/pMW219-dxs, or further MG1655/pSTV28-Ptac-IspSP/pMW219-dxs strain, respectively.

6-3) Effects of Introduction of Isoprene Synthase Derived from Various Plants on E. coli MG1655 Strain in which Expression of DXS is Enhanced

MG1655/pSTV28-Ptac-Ttrp/pMW219-dxs, MG1655/pSTV28-Ptac-IspSK/pMW219-dxs, MG1655/pSTV28-Ptac-IspSM/pMW219-dxs, or further MG1655/pSTV28-Ptac-IspSP/pMW219-dxs strain were evenly applied onto the LB plate containing 60 mg/L of chloramphenicol and 50 mg/L of kanamycin hydrochloride, and were cultured at 37° C. for 18 hours. Subsequently, the cultivation in the headspace vial was evaluated as described in Reference Example 5. The amount (μg/L) of produced isoprene and the OD value upon completion of the cultivation are described in Table 9.

TABLE 9
Amount (μg/L) of produced isoprene and OD value when the cultivation
was completed in various strains having enhanced isoprene synthase which
are prepared from E. coli MG1655 strain having enhanced DXS as host

| | | Amount |
|---|---|---|
| | | (μg/L) |
| | | of produced |
| Name of microbial strain | OD value | isoprene |
| MG1655/pSTV28-Ptac-Ttrp/pMW219-dxs | 1.46 ± 0.04 | ND |
| MG1655/pSTV28-Ptac-IspSK/pMW219-dxs | 1.13 ± 0.02 | 101 ± 28 |
| MG1655/pSTV28-Ptac-IspSM/pMW219-dxs | 1.76 ± 0.06 | 126 ± 23 |
| MG1655/pSTV28-Ptac-IspSP/pMW219-dxs | 2.21 ± 0.12 | 42 ± 17 |

From the results in Table 9, the amount of produced isoprene was larger in order of MG1655/pSTV28-Ptac-IspSM/pMW219-dxs, MG1655/pSTV28-Ptac-IspSK/pMW219-dxs, MG1655/pSTV28-Ptac-IspSP/pMW219-dxs and MG1655/pSTV28-Ptac-Ttrp/pMW219-dxs strains. From the above results, the strain introduced with the isoprene synthase derived from Mucuna also exhibited the highest ability to produce isoprene in the MEP pathway-enhanced strains.

### Reference Example 7

### Effects of Introduction of Isoprene Synthase Derived from Various Plants on E. coli MG1655 Strain in which MVA (Mevalonate) Pathway is Introduced

7-1) Cloning Gene Downstream of Mevalonate Pathway which is Derived from Yeast

A downstream region of the mevalonate pathway was obtained from Saccharomyces cerevisiae (WO2009076676, Saccharomyces Genome database http://www.yeastgenome.org/# Nucleic Acids Res., January 2012; 40: D700-D705, which is incorporated herein by reference in its entirety). An ERG12 gene encoding mevalonate kinase, an ERG8 gene encoding phosphomevalonate kinase, an ERG19 gene encoding diphosphomevalonate decarboxylase, and an IDI1 gene encoding isopentenyl-diphosphate delta isomerase were amplified by PCR with genomic DNA of S. cerevisiae as the template using the primer shown below (Table 10). Prime Star Max Premix sold by Takara Bio Inc. was used for a PCR enzyme, and the reaction was performed at 98° C. for 2 minutes and for 30 cycles of 98° C. for 10 seconds, 55° C. for 5 seconds and 72° C. for 5 seconds/kb. Cloning and construction of an expression vector were performed by introducing the PCR fragment into the pSTV28-Ptac-Ttrp vector (SEQ ID NO:17) treated with the restriction enzyme SmaI by an in-fusion cloning method. E. coli DH5α was transformed with the expression vector, clones having assumed sequence length from each gene were selected, a plasmid was extracted according to standard methods, and its sequence was confirmed. The nucleotide sequences of these amplified genes and the amino acid sequences of the enzymes encoded by these genes are available on Saccharomyces Genome database http://www.yeastgenome.org/#.

TABLE 10
Primer sequences used for cloning
of genes downstream of mevalonate pathway

| Amplified | Sequence | |
|---|---|---|
| gene | name | Sequence (5′-) |
| ERG12 | MVK-IFS_5742-33-1 | ACACAAGGAGACTCC |
| | | CATGTCATTACCGTT |
| | | CTTAACTTCT |
| | | (SEQ ID NO: 32) |
| ERG12 | MVK-IFA_5742-33-2 | GGAACTGGCGGCTCC |
| | | CGGGTTATTATGAAG |
| | | TCCATGGTAAATTCG |
| | | T |
| | | (SEQ ID NO: 33) |
| ERG8 | PMK-IFS_5742-33-3 | ACACAAGGAGACTCC |
| | | CATGTCAGAGTTGAG |
| | | AGCCTTCA |
| | | (SEQ ID NO: 34) |
| ERG8 | PMK-IFA_5742-33-4 | GGAACTGGCGGCTCC |
| | | CGGGTTATTATTTAT |
| | | CAAGATAAGTTTCCG |
| | | G |
| | | (SEQ ID NO: 35) |
| ERG19 | MVD-IFS_5742-33-5 | ACACAAGGAGACTCC |
| | | CATGACCGTTTACAC |
| | | AGCATCC |
| | | (SEQ ID NO: 36) |
| ERG19 | MVD-IFA_5742-33-6 | GGAACTGGCGGCTCC |
| | | CGGGTTATTATTCCT |
| | | TTGGTAGACCAGTCT |
| | | T |
| | | (SEQ ID NO: 37) |
| IDI1 | yIDI-IFS_5742-33-7 | ACACAAGGAGACTCC |
| | | CATGCCCCATGGTGC |
| | | AGTATC |
| | | (SEQ ID NO: 38) |
| IDI1 | yIDI-IFA_5742-33-8 | GGAACTGGCGGCTCC |
| | | CGGGTTATTATAGCA |
| | | TTCTATGAATTTGCC |
| | | TGTC |
| | | (SEQ ID NO: 39) |

### 7-2) Construction of Artificial Operon Downstream of Mevalonate Pathway

A sequence in which the gene encoding the mevalonate kinase and the gene encoding the phosphomevalonate kinase were arranged in straight was constructed by the in-fusion cloning method. The ERG12 gene encoding the mevalonate kinase and the ERG8 gene encoding the phosphomevalonate kinase were amplified by PCR with genomic DNA from Saccharomyces cerevisiae as the template using the primers shown in Table 11. KOD plus sold by Toyobo was used for the PCR enzyme, and the reaction was performed at 94° C. for 2 minutes and for 30 cycles of 94° C. for 15 seconds, 45° C. for 30 seconds and 68° C. for 1 minute/kb. The cloning and the construction of an expression vector were performed by inserting the PCR fragment into pUC118 vector treated with the restriction enzyme SmaI by the in-fusion cloning method. E. coli JM109 was transformed with the expression vector, clones having assumed sequence length of each gene were selected, a plasmid was extracted according to standard methods, and its sequence was confirmed. The produced plasmid was designated as pUC-mvk-pmk. The nucleotide sequence of pUC-mvk-pmk is represented by SEQ ID NO:40.

TABLE 11
Primer sequences used for ligating
mevalonate kinase and phosphomevalonate kinase

| Amplified | Sequence | |
|---|---|---|
| gene | name | Sequence (5′-) |
| ERG12 | KKS1-6038-2-1 | TCGAGCTCGGTACCC |
| | | ATGTCATTACCGTTC |
| | | TTAACTTCT |
| | | (SEQ ID NO: 41) |
| ERG12 | KKA1-6038-2-2 | TTAAGGGTGCAGGCC |
| | | TATCGCAAATTAGCT |
| | | TATGAAGTCCATGGT |
| | | AAATTCGT |
| | | (SEQ ID NO: 42) |
| ERG8 | KKS2-6083-2-3 | GGCCTGCACCCTTAA |
| | | GGAGGAAAAAAACAT |
| | | GTCAGAGTTGAGAGC |
| | | CTTCA |
| | | (SEQ ID NO: 43) |
| ERG8 | KKA2-6083-2-4 | CTCTAGAGGATCCCC |
| | | TTATTTATCAAGATA |
| | | AGTTTCCGG |
| | | (SEQ ID NO: 44) |

A sequence in which a gene encoding diphosphomevalonate decarboxylase and a gene encoding isopentenyl-diphosphate delta isomerase were arranged in straight was constructed by the in-fusion cloning method. The ERG19 gene encoding the diphosphomevalonate decarboxylase and the IDI1 gene encoding the isopentenyl-diphosphate delta isomerase were amplified by PCR with genomic DNA of Saccharomyces cerevisiae as the template using the primers shown in Table 12. KOD plus sold by Toyobo was used for the PCR enzyme, and the reaction was performed at 94° C. for 2 minutes and for 30 cycles of 94° C. for 15 seconds, 45° C. for 30 seconds and 68° C. for 1 minute/kb, and then at 68° C. for 10 minutes. The cloning and the construction of an expression vector were performed by inserting the PCR fragment into TWV228 vector treated with the restriction enzyme SmaI by the in-fusion cloning method. E. coli DH5α was transformed with the expression vector, clones having assumed sequence length of each gene were selected, a plasmid was extracted according to standard methods, and its sequence was confirmed. The produced plasmid was designated as pTWV-dmd-yidi. The nucleotide sequence of pTWV-dmd-yidi is represented by SEQ ID NO:45.

TABLE 12
Primer sequences used for ligating
diphosphomevalonate decarboxylase and
isopentenyl-diphosphate delta isomerase

| Amplified | Sequence | |
|---|---|---|
| gene | name | Sequence (5′-) |
| ERG19 | DyIS1- | TCGAGCTCGGTACCC |
| | 6083-2-5 | ATGACCGTTTACACA |
| | | GCATCC |
| | | (SEQ ID NO: 46) |
| ERG19 | DyIA1- | TTTTTTTACCTCCTA |
| | 6083-2-6 | AGGGCGATGCAGCGA |
| | | ATTGATCTTATTCCT |
| | | TTGGTAGACCAGTCT |
| | | T |
| | | (SEQ ID NO: 47) |
| IDI1 | DyIS2- | TAGGAGGTAAAAAAA |
| | 6083-2-7 | AATGACTGCCGACAA |
| | | CAATAGTATGCCCCA |
| | | TGGTGCAGTATC |
| | | (SEQ ID NO: 48) |
| IDI1 | DyIA2- | CTCTAGAGGATCCCC |
| | 6083-2-8 | TTATAGCATTCTATG |
| | | AATTTGCCTGTC |
| | | (SEQ ID NO: 49) |

A sequence in which the gene encoding the mevalonate kinase, the gene encoding the phosphomevalonate kinase, the gene encoding the diphosphomevalonate decarboxylase and the gene encoding the isopentenyl-diphosphate delta isomerase were arranged in straight was constructed by the in-fusion cloning method. An expression vector in which these four enzyme genes were arranged in straight was constructed by amplifying the gene encoding the mevalonate kinase and the gene encoding the phosphomevalonate kinase by PCR with pUC-mvk-pmk as the template using the primers shown in Table 13 and amplifying the gene encoding the diphosphomevalonate decarboxylase and the gene encoding the isopentenyl-diphosphate delta isomerase by PCR with pTWV-dmd-yidi as the template using the primers shown in Table 13, followed by cloning the amplified products into pTrcHis2B vector by the in-fusion cloning method. Prime Star HS DNA polymerase sold by Takara Bio Inc. was used for the PCR enzyme, and the reaction was carried out at 98° C. for 2 minutes followed by in 30 cycles of 98° C. for 10 seconds, 52° C. for 5 seconds and 72° C. for 1 minute/kb, and then at 72° C. for 10 minutes. The PCR fragment was inserted into pTrcHis2B vector treated with the restriction enzymes NcoI and PstI to construct the expression vector. E. coli JM109 was transformed with the expression vector, clones having an objective sequence length were selected, a plasmid was extracted according to standard methods, and its sequence was confirmed. The constructed expression vector was designated as pTrc-KKDyI (β). The nucleotide sequence of pTrc-KKDyI (f3) is represented by SEQ ID NO:50.

TABLE 13
Primer sequences used for amplifying
genes for constructing pTrc-KKDyI (β)

| Template | Sequence | |
|---|---|---|
| plasmid | name | Sequence (5′-) |
| pUC-mvk-pmk | KKDS2_6038- | GAGGAATAAACCATGTCA |
| | 3-2 | TTACCGTTCTTAACTTCT |
| | | (SEQ ID NO: 51) |
| pUC-mvk-pmk | KKMyIA_6038- | AAGGGCGAATTCTGCATG |
| | 2-9 | CAGCTACCTTAAGTTATT |
| | | TATCAAGATAAGTTTCCG |
| | | G (SEQ ID NO: 52) |
| pTWV-dmd- | KMS_6038- | GCAGAATTCGCCCTTAAG |
| yidi | 6-1 | GAGGAAAAAAAAATGACC |
| | | GTTTACACAGCATCC |
| | | (SEQ ID NO: 53) |
| pTWV-dmd- | KDyIA_6038- | CCATATGGTACCAGCTGC |
| yidi | 3-3 | AGTTATAGCATTCTATGA |
| | | ATTTGCCTGTC |
| | | (SEQ ID NO: 54) |

### 7-3) Fixation of Downstream Region of Mevalonate Pathway on Chromosome

The sequence in which the gene encoding the mevalonate kinase, the gene encoding the phosphomevalonate kinase, the gene encoding the diphosphomevalonate decarboxylase and the gene encoding the isopentenyl-diphosphate delta isomerase were arranged in straight was expressed on a chromosome. A glucose isomerase promoter was used for the expression of the gene, and a transcription termination region of aspA gene in E. coli was used for the termination of the transcription (WO2010031062, which is incorporated herein by reference in its entirety). A translocation site of Tn7 was used as a chromosomal site to be fixed (Mol Gen Genet., 1981; 183 (2): 380-7, which is incorporated herein by reference in its entirety). A cat gene was used as a drug marker after the fixation of the chromosome. A Tn7 downstream region in the chromosome region to be fixed was amplified by PCR with genomic DNA of E. coli as the template using the primers shown in Table 14. Prime Star HS DNA polymerase sold by Takara Bio Inc. was used for the PCR enzyme, and the reaction was carried out at 98° C. for 2 minutes followed by in 30 cycles of 98° C. for 10 seconds, 52° C. for 5 seconds and 72° C. for 1 minute/kb, and then at 72° C. for 10 minutes. A cat gene region containing a λ phage attachment site was amplified by PCR with pMW118-attL-Cm-attR plasmid as the template using the primers shown in Table 14 (WO2010-027022). Prime Star HS DNA polymerase sold by Takara Bio Inc. was used for the PCR enzyme, and the reaction was carried out at 95° C. for 3 minutes followed by in 2 cycles of 95° C. for 1 minute, 34° C. for 30 seconds and 72° C. for 40 seconds, 2 cycles of 95° C. for 30 seconds, 50° C. for 30 seconds and 72° C. for 40 seconds, and then at 72° C. for 5 minutes. A sequence downstream of the mevalonate pathway to which a promoter and a transcription termination region had been added (hereinafter abbreviated as KKDyI) was amplified with pTrc-KKDyI (β) as the template using the primers shown in Table 14. Prime Star HS DNA polymerase sold by Takara Bio Inc. was used for the PCR enzyme, and the reaction was carried out at 98° C. for 2 minutes followed by in 30 cycles of 98° C. for 10 seconds, 52° C. for 5 seconds and 72° C. for 1 minute/kb, and then at 72° C. for 10 minutes. A vector was constructed using these PCR products and pMW219 treated with the restriction enzyme SmaI by the in-fusion cloning method. E. coli JM109 was transformed with the expression vector, clones having an objective sequence length were selected, a plasmid was extracted according to standard methods, and its sequence was confirmed. The resulting plasmid was designated as pMW219-KKDyI-TaspA. The nucleotide sequence of pMW219-KKDyI-TaspA is represented by SEQ ID NO:55.

Subsequently, a Tn7 upstream region in the chromosome region to be fixed was amplified by PCR with the genomic DNA of E. coli as the template using the primers shown in Table 14. Prime Star HS DNA polymerase sold by Takara Bio Inc. was used for the PCR enzyme, and the reaction was carried out at 98° C. for 2 minutes followed by in 30 cycles of 98° C. for 10 seconds, 52° C. for 5 seconds and 72° C. for 1 minute/kb, and then at 72° C. for 10 minutes. A vector was constructed using the PCR product and pMW219-KKDyI-TaspA treated with the restriction enzyme SalI by the in-fusion cloning method. E. coli JM109 was transformed with the expression vector, clones having an objective sequence length were selected, a plasmid was extracted according to standard methods, and its sequence was confirmed. The resulting plasmid was designated as pMW-Tn7-Pgi-KKDyI-TaspA-Tn7. The sequence of the constructed plasmid is represented by SEQ ID NO:56.

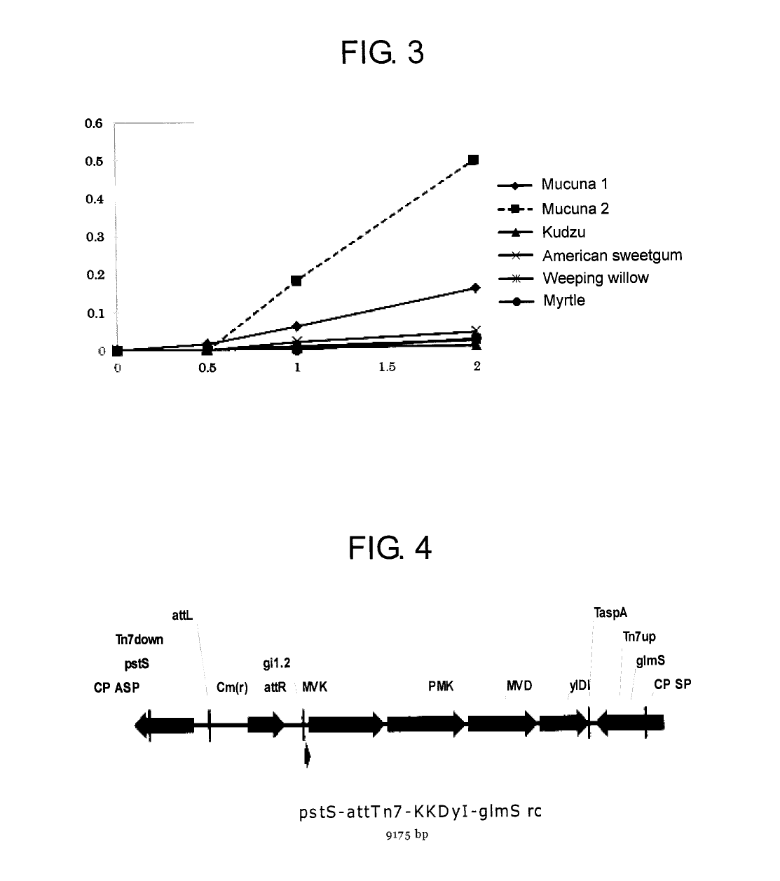

Subsequently, a chromosome having a region including the chloramphenicol resistance gene, the glucose isomerase promoter, the operon downstream of the mevalonate pathway, and the aspA gene transcription termination region was fixed using λ-Red method. A fragment for chromosome fixation was prepared by extracting the plasmid pMW-Tn7-Pgi-KKDyI-TaspA-Tn7 and then treating it with the restriction enzymes PvuI and SalI followed by purifying it. E. coli MG1655 containing a plasmid pKD46 having a temperature-sensitive replication capacity (hereinafter referred to as MG1655/pKD46) was used for the electroporation. The plasmid pKD46 (Proc. Natl. Acad. Sci. USA, 2000, vol. 97, No. 12, p 6640-6645, which is incorporated herein by reference in its entirety) contains a DNA fragment of total 2154 nucleotides (GenBank/EMBL Accession No. J02459, 31088th to 33241st) of λ phage containing λ Red system genes (λ, β, exo genes) controlled by an arabinose-inducible ParaB Promoter. After the electroporation, a colony that had acquired the resistance to chloramphenicol was obtained, subsequently genomic DNA was extracted, and it was confirmed by PCR using the primers shown in Table 16 that the objective region was fixed on the chromosome. Further, the sequence of the objective region was confirmed by confirming the sequence of the PCR fragment. The nucleotide sequence of the mevalonate pathway downstream and its proximal region fixed on the chromosome is represented by SEQ ID NO:57, and its construction outline is shown in FIG. 4. The resulting mutant was designated as MG1655 cat-Pgi-KKDyI.

The drug marker in MG1655 cat-Pgi-KKDyI was removed by the following procedure. Competent cells of MG1655 cat-Pgi-KKDyI were made, and then pMW-int-xis was introduced therein. pMW-int-xis is a plasmid containing a gene encoding integrase (Int) of the λ phage and a gene encoding excisionase (Xis) of the λ phage and having the temperature-sensitive replication capacity (WO2007/037460, JP Publication No. 2005-058827, both of which are incorporated herein by reference in their entireties).

The chloramphenicol-resistant gene located in a region sandwiched with attL and attR that are the attachment site of the λ phage is dropped off from the chromosome by introducing pMW-int-xis. As a result, it is known that the host loses the resistance to chloramphenicol. And, a chloramphenicol-sensitive strain was obtained from the resulting colony, and subsequently cultured on the LB medium at 42° C. for 6 hours. The cultured microbial cells were applied onto the LB plate medium to allow colonies to appear. A colony that had lost the resistance to ampicillin was selected from these colonies to remove the drug resistance. The mutant obtained as above was designated as MG1655 Pgi-KKDyI.

TABLE 14
Primers for making PCR fragments used for
construction of pMW219-KKDyI-TaspA

| Template | Amplified | Sequence | |
|---|---|---|---|
| DNA | region | name | Sequence (5′-) |
| E. coli | Tn7 | Tn7dS_6038- | TCGAGCTCGGTACCC |
| genome | down- | 7-1 | TGTTTTTCCACTCTT |
| | stream | | CGTTCACTTT |
| | | | (SEQ ID NO: 58) |
| E. coli | Tn7 | Tn7dA_6038- | AGGCTTCATTTTAAT |
| genome | down- | 7-2 | CAAACATCCTGCCAA |
| | stream | | CTC |
| | | | (SEQ ID NO: 59) |
| pMW-attL- | attL- | Tn7dattLcmS_ | ATTAAAATGAAGCCT |
| Cm-attR | cat- | 6038-7-4 | GCTTTTTTAT |
| | attR | | (SEQ ID NO: 60) |
| pMW-attL- | attL- | PgiattRcmA_ | GGCATCGTCAAGGGC |
| Cm-attR | cat- | 6038-7-5 | CGCTCAAGTTAGTAT |
| | attR | | AA |
| | | | (SEQ ID NO: 61) |
| pTrc- | KKDyI | gi1.2-MVK- | GCCCTTGACGATGCC |
| KKDyI(β) | | S_6038-7-6 | ACATCCTGAGCAAAT |
| | | | AATTCAACCACTAAT |
| | | | TGTGAGCGGATAACA |
| | | | CAAGGAGGAAACAGC |
| | | | TATGTCATTACCGTT |
| | | | CTTAACTTC |
| | | | (SEQ ID NO: 62) |
| pTrc- | KKDyI | pMW-TaspA- | CTCTAGAGGATCCCC |
| KKDyI(β) | | yIDIA_6038- | GGCCCCAAGAAAAAA |
| | | 7-7 | GGCACGTCATCTGAC |
| | | | GTGCCTTTTTTATTT |
| | | | GTAGACGCGTTGTTA |
| | | | TAGCATTCTATGAAT |
| | | | TTGCCT |
| | | | (SEQ ID NO: 63) |

TABLE 15
Primers for making PCR fragments used for
construction of pMW-Tn7-Pgi-KKDyI-TaspA-Tn7

| Template | Amplified | | |
|---|---|---|---|
| DNA | region | Sequence name | Sequence (5′-) |
| E. coli | Tn7 | Tn7upSv02_6038- | ATCCTCTAGAGTCGA |
| genome | upstream | 24-1 | AAGAAAAATGCCCCG |
| | | | CTTACG |
| | | | (SEQ ID NO: 64) |
| E. coli | Tn7 | Tn7upAv02_6038- | ATGCCTGCAGGTCGA |
| genome | upstream | 24-2 | CTGTCACAGTCTGGC |
| | | | GAAACCG |
| | | | (SEQ ID NO: 65) |

TABLE 16
PCR primers for confirming chromosome
fixation of mevalonate pathway downstream

| Sequence name | Sequence (5′-) |
|---|---|
| Tn7v02-F_6038-22-5 | ACGAACTGCTGTCGAAGGTT |
| | (SEQ ID NO: 66) |
| Tn7v02-R_6038-22-6 | GGTGTACGCCAGGTTGTTCT |
| | (SEQ ID NO: 67) |

### 7-4) Substitution of Promoter Downstream of Mevalonate Pathway on Chromosome

The promoter of the operon downstream of the mevalonate pathway on the chromosome was substituted by the λ-red method. A genomic fragment having attL-Tet-attR-Ptac was used as the template for PCR. This is one in which the tac promoter, and attL and attR that are the attachment sites for a tetracycline resistant drug marker and the λ phage are aligned. This sequence is represented by SEQ ID NO:68. A PCR fragment was prepared using the promoter shown in Table 17. LA-Taq polymerase sold by Takara Bio Inc. was used for the PCR enzyme, and the reaction was carried out at 92° C. for 1 minute, then for 40 cycles of 92° C. for 10 seconds, 50° C. for 20 seconds and 72° C. for 1 minute/kb, and further at 72° C. for 7 minutes. The PCR product was purified. MG1655 Pgi-KKDyI containing the plasmid pKD46 (hereinafter referred to as MG1655 Pgi-KKDyI/pKD46) having the temperature-sensitive replication capacity was used for the electroporation. The plasmid pKD46 (Proc. Natl. Acad. Sci. USA, 2000, vol. 97, No. 12, p 6640-6645, which is incorporated herein by reference in its entirety) contains a DNA fragment of total 2154 nucleotides (GenBank/EMBL Accession No. J02459, 31088th to 33241st) of λ phage containing λ Red system genes (λ, β, exo genes) controlled by an arabinose-inducible ParaB Promoter. The plasmid pKD46 is required for incorporating the PCR product into MG1655 Pgi-KKDyI.

Competent cells for the electroporation were prepared as follows. MG1655 Pgi-KKDyI/pKD46 cultured in the LB medium containing 100 mg/L of ampicillin at 30° C. overnight were diluted to 100 times with 5 mL of LB medium containing ampicillin and L-arabinose (1 mM). The resulting cells in diluted suspension were grown until OD600 reached about 0.6 with ventilating at 30° C., and subsequently washed three times with ice-cold 10% glycerol solution to use for the electroporation. The electroporation was performed using 50 μL of the competent cells and about 100 ng of the PCR product. The cells after the electroporation in 1 mL of SOC medium (Molecular Cloning: Laboratory Manuals, 2nd Edition, Sambrook, J. et al., Cold Spring Harbor Laboratory Press (1989), which is incorporated herein by reference in its entirety) were cultured at 37° C. for one hour, and subjected to a plate culture on LB agar medium at 37° C. to select a chloramphenicol-resistant transformant. Subsequently, in order to remove the pKD46 plasmid, the transformant was subcultured on the LB agar medium containing tetracycline at 37° C. The ampicillin resistance was examined in the obtained colonies, and an ampicillin-resistant strain having no pKD46 was obtained. A mutant containing the tac promoter substitution that could be distinguished by the tetracycline-resistant gene was obtained. The obtained mutant was designated as MG1655 tet-Ptac-KKDyI.

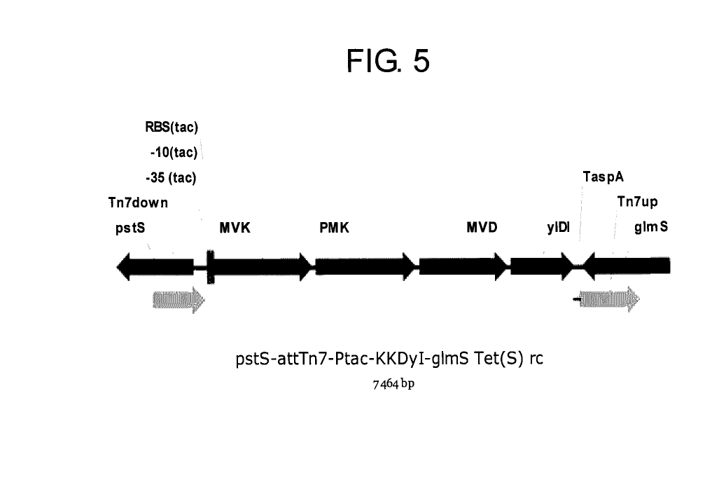

The antibiotic marker was removed by the following procedure. Competent cells of MG1655 tet-Ptac-KKDyI were made, and then pMW-int-xis was introduced therein. pMW-int-xis is a plasmid containing the genes encoding integrase (Int) and excisionase (Xis) of the λ phage and having the temperature-sensitive replication capacity (WO2007/037460, JP Publication No. 2005-058827, both of which are incorporated herein by reference in their entireties). The tetracycline-resistant gene located in a region sandwiched with attL and attR that are the attachment site of the λ phage is dropped off from the chromosome by introducing pMW-int-xis. As a result, it is known that the host loses the resistance to tetracycline. Thus, a tetracycline-sensitive strain was obtained from the resulting colonies. Cells of this strain were cultured on the LB medium at 42° C. for 6 hours, and the cultured cells were applied onto the LB plate medium to allow colonies to appear. A clone that had lost the resistance to ampicillin was selected to remove the drug resistance. The resulting mutant was designated as MG1655 Ptac-KKDyI. The nucleotide sequence of the mevalonate pathway downstream and its proximal region controlled by the tac promoter on the chromosome is represented by SEQ ID NO:69, and its outline is shown in FIG. 5.

TABLE 17
Primers for making PCR
fragments for promoter substitution

| Sequence name | Sequence (5′-) |
|---|---|
| APtacKKDyIv03_6038-36-5 | gataaagtcttcagtctgatttaaat |
| | aagcgttgatattcagtcaattactg |
| | aagcctgcttttttatac |
| | (SEQ ID NO: 70) |
| SPtacKKDyIv02_6038-36-3 | tcaccaaaaataataacctttcccgg |
| | tgcagaagttaagaacggtaatgaCA |
| | Tggcagtctccttgtgtga |
| | (SEQ ID NO: 71) |

7-5) Introduction of Isoprene Synthase Derived from Various Plants into MG1655 Ptac-KKDyI Strain

Competent cells of MG1655 Ptac-KKDyI strain were prepared, and then pSTV28-Ptac-Ttrp, pSTV28-Ptac-IspSK, pSTV28-Ptac-IspSM, or further pSTV28-Ptac-SP was introduced therein. The cells were evenly applied onto the LB plate containing 60 mg/L of chloramphenicol, and the cells were cultured at 37° C. for 18 hours. Transformants that exhibited the chloramphenicol resistance were obtained from the resulting plate. A strain in which pSTV28-Ptac-Ttrp, pSTV28-Ptac-IspSK, pSTV28-Ptac-IspSM, or pSTV28-Ptac-IspSP had been introduced into MG1655 Ptac-KKDyI strain was designated as MG1655 Ptac-KKDyI/pSTV28-Ptac-Ttrp, MG1655 Ptac-KKDyI/pSTV28-Ptac-IspSK, MG1655 Ptac-KKDyI/pSTV28-Ptac-IspSM, or MG1655 Ptac-KKDyI/pSTV28-Ptac-IspSP, respectively.

7-6) Effects of Introduction of Isoprene Synthase Derived from Various Plants on MG1655 Strain in which MVA Pathway is Enhanced

Microbial cells of MG1655 Ptac-KKDyI/pSTV28-Ptac-Ttrp, MG1655 Ptac-KKDyI/pSTV28-Ptac-IspSK, MG1655 Ptac-KKDyI/pSTV28-Ptac-IspSM, or further MG1655 Ptac-KKDyI/pSTV28-Ptac-IspSP strain were evenly applied onto the LB plate containing 60 mg/L of chloramphenicol, and the cells were cultured at 37° C. for 18 hours. One loopful of the microbial cells from the resulting LB plate was inoculated to 1 mL of M9 glucose (containing mevalonic acid) medium in a headspace vial (22 mL CLEAR CRIMP TOP VIAL (cat #B0104236) manufactured by Perkin Elmer), and subsequently the cultivation was evaluated according to the method described in Reference Example 2. A composition of the M9 glucose (containing mevalonic acid) medium is described in Table 18. The amount of produced isoprene and the OD value upon completion of the cultivation are described in Table 19.

TABLE 18
Composition of M9 glucose (containing mevalonic acid) medium

| | |
|---|---|
| Glucose | 2.0 g/L |
| Na₂HPO₄ | 6.0 g/L |
| KH₂PO₄ | 3.0 g/L |
| NaCl | 0.5 g/L |
| NH₄Cl | 1.0 g/L |
| Mevalonic acid (manufactured by ADEKA) | 1.0 g/L |
| 1M MgSO₄ (autoclaved) | 1.0 mL |
| 1M CaCl₂ (autoclaved) | 0.1 mL |

Chloramphenicol was added at a final concentration of 60 mg/L.

A total volume was adjusted to 1 L, and the medium was sterilized by filtration.

TABLE 19
Amount (mg/L) of produced isoprene and OD value when cultivation of
MG1655 Ptac-KKDyI/pSTV28-Ptac-Ttrp, MG1655 Ptac-KKDyI/
pSTV28-Ptac-IspSK, MG1655 Ptac-KKDyI/pSTV28-Ptac-IspSM, or
further MG1655 Ptac-KKDyI/pSTV28-Ptac-IspSP was completed

| | | Amount |
|---|---|---|
| | | (mg/L) |
| | | of produced |
| Name of microbial strain | OD value | isoprene |
| MG1655 Ptac-KKDyI/pSTV28-Ptac-Ttrp | 2.08 ± 0.07 | 0.07 ± 0.01 |
| MG1655 Ptac-KKDyI/ | 2.48 ± 0.13 | 30.96 ± 3.04 |
| pSTV28-Ptac-IspSK | | |
| MG1655 Ptac-KKDyI/ | 2.48 ± 0.09 | 57.13 ± 15.00 |
| pSTV28-Ptac-IspSM | | |
| MG1655 Ptac-KKDyI/pSTV28-Ptac-IspSP | 1.95 ± 0.09 | 0.52 ± 0.01 |

From the results in Table 19, the amount of produced isoprene was larger in order of MG1655 Ptac-KKDyI/pSTV28-Ptac-IspSM, MG1655 Ptac-KKDyI/pSTV28-Ptac-IspSK, MG1655 Ptac-KKDyI/pSTV28-Ptac-IspSP, and MG1655 Ptac-KKDyI/pSTV28-Ptac-Ttrp strains. From the above results, the strain introduced with the isoprene synthase derived from Mucuna also exhibited the highest ability to produce isoprene in the strains introduced with the MVA pathway.

### Example 5

### Construction of P. ananatis AG10265

(5-1) Construction of pMW-Para-mvaES-Ttrp
(5-1-1) Chemical Synthesis of mvaE Gene Derived from Enterococcus faecalis

The nucleotide sequence and amino acid sequence of mvaE derived from Enterococcus faecalis and encoding acetyl-CoA acetyltransferase and hydroxymethlglutaryl-CoA reductase have been already known (ACCESSION number of nucleotide sequence: AF290092.1 (1479 . . . 3890), ACCESSION number of amino acid sequence: AAG02439) (J. Bacteriol. 182(15), 4319-4327(2000), which is incorporated herein by reference in its entirety). The amino acid sequence of the mvaE protein derived from Enterococcus faecalis and the nucleotide sequence of its gene are shown in SEQ ID NO:72 and SEQ ID NO:73, respectively. In order to efficiently express the mvaE gene in E. coli, a mvaE gene in which codon usage was optimized for E. coli was designed and designated as EFmvaE. Its nucleotide sequence is shown in SEQ ID NO:74. This mvaE gene was chemically synthesized, cloned into pUC57 (supplied from GenScript), and the resulting plasmid was designated as pUC57-EFmvaE.

(5-1-2) Chemical Synthesis of mvaS Gene Derived from Enterococcus faecalis

The nucleotide sequence and amino acid sequence of mvaS derived from Enterococcus faecalis and encoding hydroxymethylglutaryl-CoA synthase have been already known (ACCESSION number of nucleotide sequence: AF290092.1, complement(142 . . . 1293), ACCESSION number of amino acid sequence: AAG02438) (J. Bacteriol. 182(15), 4319-4327 (2000), which is incorporated herein by reference in its entirety). The amino acid sequence of the mvaS protein derived from Enterococcus faecalis and the nucleotide sequence of its gene are shown in SEQ ID NO:75 and SEQ ID NO:76, respectively. In order to efficiently express the mvaS gene in E. coli, a mvaS gene in which codon usage was optimized for E. coli was designed and designated as EFmvaS. Its nucleotide sequence is shown in SEQ ID NO:77. This mvaS gene was chemically synthesized, cloned into pUC57 (supplied from GenScript), and the resulting plasmid was designated as pUC57-EFmvaS.

(5-1-3) Construction of Arabinose-Inducible mvaES Expression Vector

An arabinose-inducible expression vector for a gene upstream of a mevalonate pathway was constructed by the following procedure. A PCR fragment comprising Para composed of araC and araBAD promoter sequences derived from E. coli was obtained by PCR using the plasmid pKD46 as a template and using synthesized oligonucleotides represented by SEQ ID NO:78 and SEQ ID NO:79 as primers. A PCR fragment comprising the EFmvaE gene was obtained by PCR using the plasmid pUC57-EFmvaE as the template and using synthesized oligonucleotides represented by SEQ ID NO:80 and SEQ ID NO:81 as the primers. A PCR fragment comprising the EFmvaS gene was obtained by PCR using the plasmid pUC57-EFmvaS as the template and using synthesized oligonucleotides represented by SEQ ID NO:82 and SEQ ID NO:83 as the primers. A PCR fragment comprising a Ttrp sequence was obtained by PCR using the plasmid pSTV-Ptac-Ttrp as the template and using synthesized oligonucleotides represented by SEQ ID NO:84 and SEQ ID NO:85 as the primers. PrimeStar polymerase (supplied from TaKaRa Bio) was used for PCR for obtaining these 4 PCR fragments. A reaction solution was prepared according to a composition attached to the kit, and the reaction by a cycle of 98° C. for 10 seconds, 55° C. for 5 seconds and 72° C. for one minute/kb was repeated 30 times. Using the purified PCR product comprising Para and the purified PCR product comprising the EFmvaE gene as the template, PCR was carried out using the synthesized oligonucleotides represented by SEQ ID NO:78 and SEQ ID NO:81 as the primers. Using the purified PCR product comprising the EFmvaS gene and the purified PCR product comprising Ttrp as the template, PCR was carried out using the synthesized oligonucleotides represented by SEQ ID NO:82 and SEQ ID NO:85 as the primers. As a result, the PCR product comprising Para and the EFmvaE gene and the PCR product comprising the EFmvaS gene and Ttrp were obtained. A plasmid pMW219 (supplied from Nippon Gene) was digested with SmaI according to the standard method. After digestion with SmaI, pMW219, the purified PCR product comprising Para and the EFmvaE gene and the purified PCR product comprising the EFmvaS gene and Ttrp were ligated using In-Fusion HD Cloning Kit (supplied from Clontech). The resulting plasmid was designated as pMW-Para-mvaES-Ttrp.

(5-2) Construction of pTrc-KKDyI-ispS(K)

First, an expression vector comprising a sequence where mevalonate kinase, phosphomevalonate kinase, diphosphomevalonate decarboxylase and isopentenyl diphosphate delta isomerase were aligned in line was constructed by In-fusion cloning. Sequences of mevalonate kinase and phosphomevalonate kinase were amplified by PCR using pUC-mvk-pmk (see Reference Example 7-2) (SEQ ID NO:40) as the template and using primers consisting of the nucleotide sequences of SEQ ID NOs:86 to 89. Sequences of diphosphomevalonate decarboxylase and isopentenyl diphosphate delta isomerase were amplified by PCR using pTWV-dmd-yidi (see Reference Example 7-2) ((SEQ ID NO:45) as the template and using primers consisting of the nucleotide sequences of SEQ ID NOs:86 to 89. Subsequently, the expression vector where these four enzyme genes were aligned in line was constructed by cloning them into pTrcHis2B vector using the in-fusion cloning method. PrimeStar HS DNA polymerase sold by TaKaRa Bio was used for PCR, and the reaction was carried out under the condition of 98° C. for 2 minutes, 30 cycles (98° C. for 10 seconds, 52° C. for 5 seconds and 72° C. for one minute/kb) and 72° C. for 10 minutes. The resulting PCR fragment was inserted into the pTrcHis2B vector digested with restriction enzymes NcoI and PstI using the in-fusion cloning method to construct the expression vector. E. coli JM109 was transformed with this expression vector, a clone having an objective length was selected, subsequently the plasmid was extracted according to the standard methods, and its sequence was confirmed. The constructed expression vector was designated as pTrc-KKDyI(α). A nucleotide sequence of pTrc-KKDyI(α) is shown in SEQ ID NO:90.

Then, a plasmid pTrc-KKDyI-ispS(K) where IspS(K) was added to the obtained expression vector pTrc-KKDyI(α) (SEQ ID NO:90) was constructed by the following procedure. pTrc-KKDyI(α) was digested with the restriction enzyme PstI (supplied from TaKaRa Bio) to obtain pTrc-KKDyI(α)/PstI. Using pUC57-ispSK as the template, PCR was carried out using pTrcKKDyIkSS_6083-10-1 (SEQ ID NO:91) and pTrcKKDyIkSA_6083-10-2 (SEQ ID NO:92) as the primers and using PrimeStar polymerase (supplied from TaKaRa Bio). A reaction solution was prepared according to the composition attached to the kit, and the reaction of a cycle of 98° C. for 10 seconds, 54° C. for 20 seconds and 68° C. for 120 seconds was repeated 30 times. As a result, a PCR product comprising an IspSK gene was obtained. Subsequently, the purified IspSK gene fragment and pTrc-KKDyI(α)/PstI were ligated using In-Fusion HD Cloning Kit (supplied from Clontech). The resulting plasmid was designated as pTrc-KKDyI-ispS(K) (SEQ ID NO:93).

(5-3) Construction of pSTV-Ptac-ispS-Mmamvk
(5-3-1) Chemical Synthesis of Mevalonate Kinase Derived from Methanosarcina mazei

The nucleotide sequence and amino acid sequence of mevalonate kinase derived from Methanosarcina mazei Go1 have been already known (ACCESSION number of nucleotide sequence: NC_003901.1 (2101873 . . . 2102778, LOCUS TAG MM_1762, ACCESSION number of amino acid sequence: NP_633786.1)). The amino acid sequence of the MVK protein derived from Methanosarcina mazei and the nucleotide sequence of its gene are shown in SEQ ID NO:94 and SEQ ID NO:95, respectively. In order to efficiently express the MVK gene in E. coli, the MVK gene in which the codon usage was optimized for E. coli was designed and designated as Mmamvk. A nucleotide sequence of Mmamvk is shown in SEQ ID NO:96. The Mmamvk gene was chemically synthesized, cloned into pUC57 (supplied from GenScript), and the resulting plasmid was designated as pUC57-Mmamvk.

(5-3-2) Construction of Plasmid for Expressing Isoprene Synthase Gene Derived from Pueraria montana Var. Lobata (Kudzu) and MVK Gene

A plasmid for expressing the IspSK gene and the Mmamvk gene in E. coli was constructed by the following procedure. Using pUC57-IspSK as the template, PCR was carried out using synthesized oligonucleotide consisting of the nucleotide sequences of SEQ ID NO:97 and SEQ ID NO:98 as the primers and using PrimeStar polymerase (supplied from TaKaRa Bio). A reaction solution was prepared according to the composition attached to the kit, and the reaction of a cycle of 98° C. for 10 seconds, 54° C. for 20 seconds and 68° C. for 120 seconds was repeated 40 times. As a result, a PCR product comprising the IspSK gene was obtained. Likewise, using pSTV28-Ptac-Ttrp as the template, PCR was carried out using synthesized oligonucleotide consisting of the nucleotide sequences of SEQ ID NO:99 and SEQ ID NO:100 as the primers and using PrimeStar polymerase (supplied from TaKaRa Bio). A reaction solution was prepared according to the composition attached to the kit, and the reaction of a cycle of 98° C. for 10 seconds, 54° C. for 20 seconds and 68° C. for 210 seconds was repeated 40 times. As a result, a PCR product comprising pSTV28-Ptac-Ttrp was obtained. Subsequently, the IspSK gene fragment and the PCR product comprising pSTV28-Ptac-Ttrp were ligated using In-Fusion HD Cloning Kit (supplied from Clontech). The resulting plasmid for expressing the IspSK gene was designated as pSTV28-Ptac-IspSK. Then, using pUC57-Mmamvk as the template, PCR was carried out using synthesized oligonucleotide consisting of the nucleotide sequences of SEQ ID NO:101 and SEQ ID NO:102 as the primers and using PrimeStar polymerase (supplied from TaKaRa Bio). A reaction solution was prepared according to the composition attached to the kit, and the reaction of a cycle of 98° C. for 10 seconds, 55° C. for 5 seconds and 72° C. for one minute/kb was repeated 30 times. As a result, a PCR product comprising the Mmamvk gene was obtained. Likewise, using pSTV28-Ptac-IspSK as the template, PCR was carried out using synthesized oligonucleotide consisting of the nucleotide sequences of SEQ ID NO:103 and SEQ ID NO:104 as the primers and using PrimeStar polymerase (supplied from TaKaRa Bio). A reaction solution was prepared according to the composition attached to the kit, and the reaction of a cycle of 98° C. for 10 seconds, 55° C. for 5 seconds and 72° C. for one minute/kb was repeated 30 times. As a result, a PCR product comprising pSTV28-Ptac-IspSK was obtained. Subsequently, the purified Mmamvk gene and the PCR product comprising pSTV28-Ptac-IspSK were ligated using In-Fusion HD Cloning Kit (supplied from Clontech). The resulting plasmid for expressing the IspSK gene and the Mmamvk gene was designated as pSTV28-Ptac-IspSK-Mmamvk.

(5-4) Construction of pMW-Ptac-Mclmvk-Ttrp

Next, a plasmid for expressing MVK (PMW-Ptac-mvk-Ttrp) was constructed.

Using pUC57-Mclmvk as the template, PCR was carried out using synthesized oligonucleotides consisting of the nucleotide sequences of Mcl_mvk_N (SEQ ID NO:105) and Mcl_mvk_C (SEQ ID NO:106) as the primers and using PrimeStar polymerase (supplied from TaKaRa Bio). A reaction solution was prepared according to the composition attached to the kit, and the reaction of a cycle of 98° C. for 10 seconds, 55° C. for 5 seconds and 72° C. for one minute/kb was repeated 30 times. As a result, a PCR product comprising the Mclmvk gene was obtained. Likewise, using pMW219-Ptac-Ttrp (see WO2013069634A1) as the template, PCR was carried out using synthesized oligonucleotides consisting of the nucleotide sequences of PtTt219f (SEQ ID NO:107) and PtTt219r (SEQ ID NO:108) as the primers and using PrimeStar polymerase (supplied from TaKaRa Bio). A reaction solution was prepared according to the composition attached to the kit, and the reaction of a cycle of 98° C. for 10 seconds, 55° C. for 5 seconds and 72° C. for one minute/kb was repeated 30 times. As a result, a PCR product comprising pMW219-Ptac-Ttrp was obtained. Subsequently, the purified Mclmvk gene and the PCR product comprising pMW219-Ptac-Ttrp were ligated using In-Fusion HD Cloning kit (supplied from Clontech). The resulting plasmid for expressing the Mclmvk gene was designated as pMW-Ptac-Mclmvk-Ttrp.

### (5-5) Integrative Conditional Replication Plasmid Having Upstream Gene and Downstream Gene of Mevalonate Pathway

In order to construct an integrative plasmid possessing an upstream gene and a downstream gene of the mevalonate pathway, a vector pAH162-λattL-TcR-λattR (Minaeva N I et al., BMC Biotechnol. 2008; 8:63, which is incorporated herein by reference in its entirety) was used.

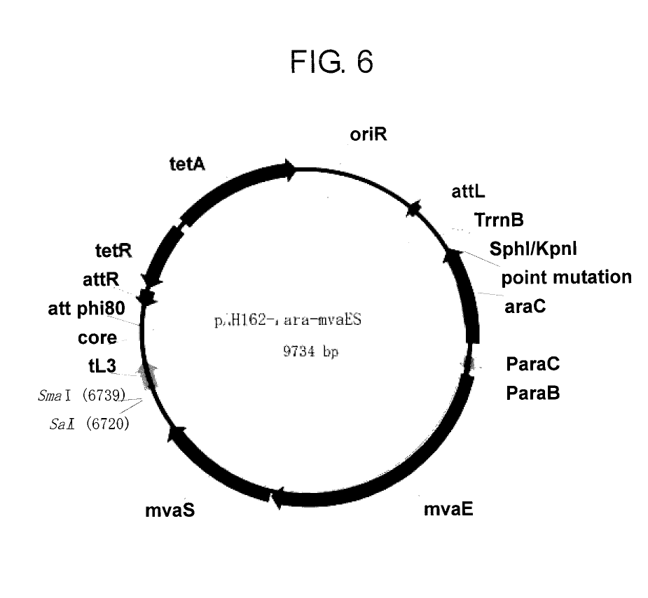

A KpnI-SalI fragment of pMW-Para-mvaES-Ttrp was cloned into a recognition site for SphI-SalI in pAH162-λattL-TcR-λattR. As a result, a plasmid pAH162-Para-mvaES possessing an operon mvaES derived from E. faecalis under the control of the Para promoter and a repressor gene AraC from E. coli was constructed (FIG. 6).

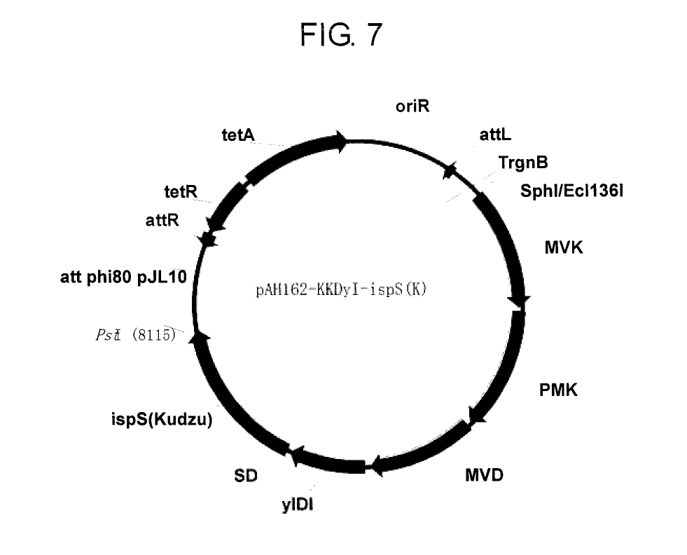

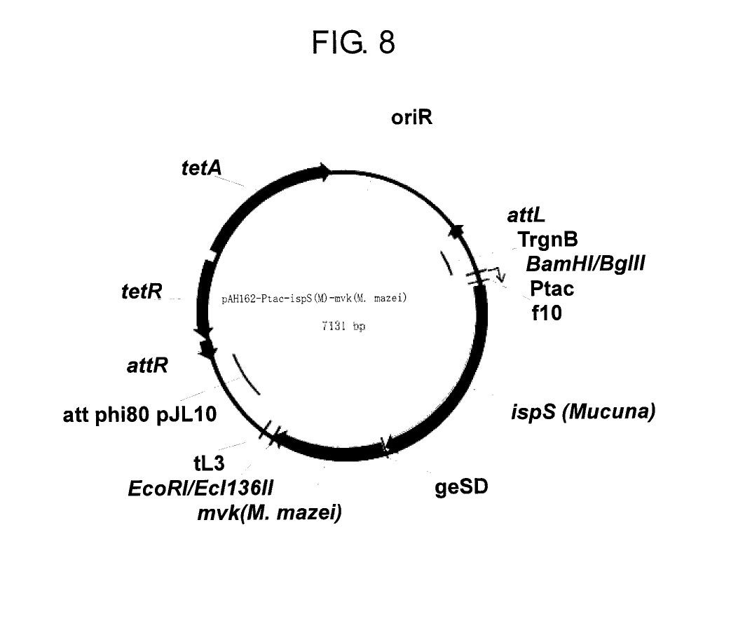

An Ec1136II-SalI fragment of a plasmid pTrc-KKDyI-ispS(K) comprising a mevalonate kinase gene, a phosphomevalonate kinase gene, a diphosphomevalonate decarboxylase gene and an IPP isomerase gene derived from S. cerevisiae and a coding portion of the ispS gene derived from kudzu was subcloned into a SphI-SalI site of pAH162-λattL-TcR-λattR, and the resulting plasmid was designated as pAH162-KKDyI-ispS(K) (FIG. 7).

A BglII-EcoRI fragment of pSTV28-Ptac-ispS-Mmamvk comprising the ispS gene (Mucuna) and the mvk gene (M. mazei) under the control of Ptac was subcloned into a recognition site for BamHI-Ec1136II in the integrative vector pAH162-λattL-TcR-λattR. The resulting plasmid pAH162-Ptac-ispS(M)-mvk(Mma) is shown in FIG. 8.

(5-6) Construction of P. ananatis SC17(0) Derivative Possessing attB Site of Phi80 Phage at Different Site in Genome

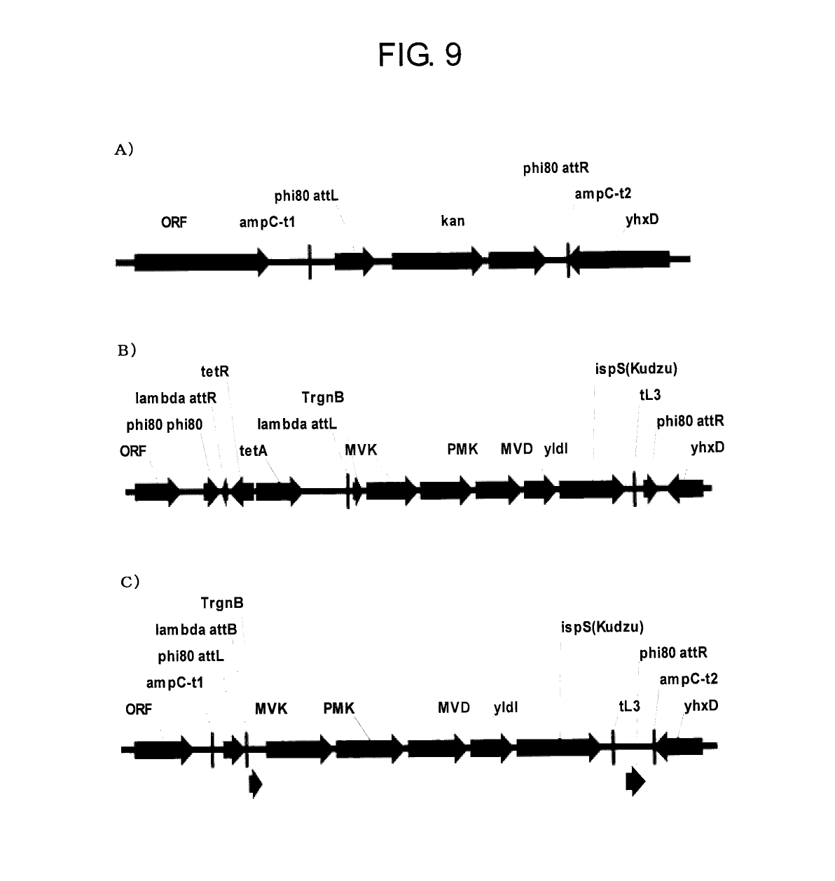

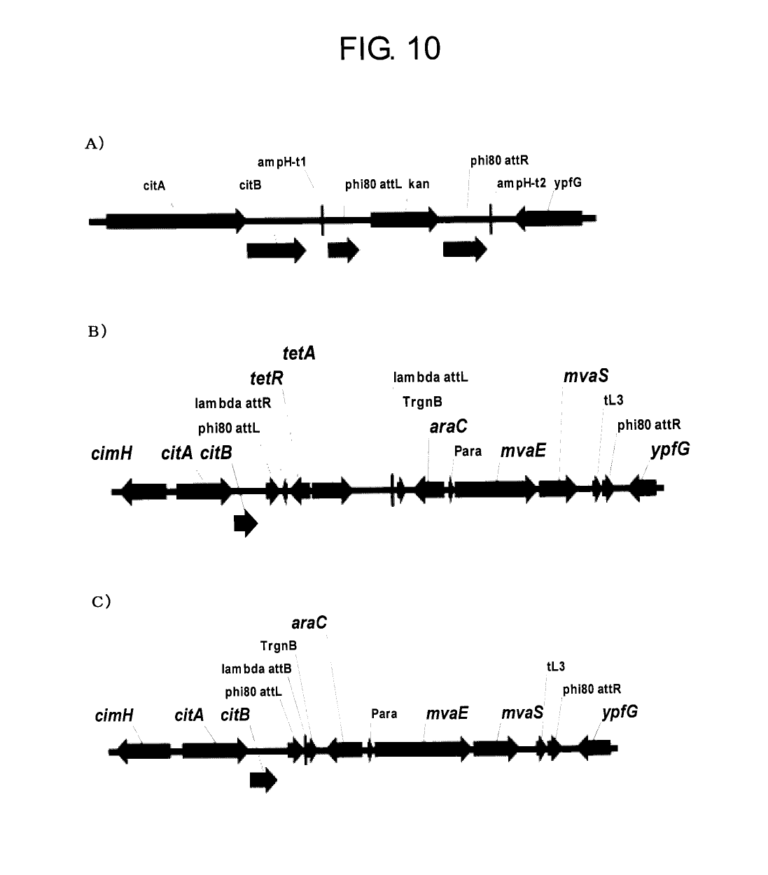

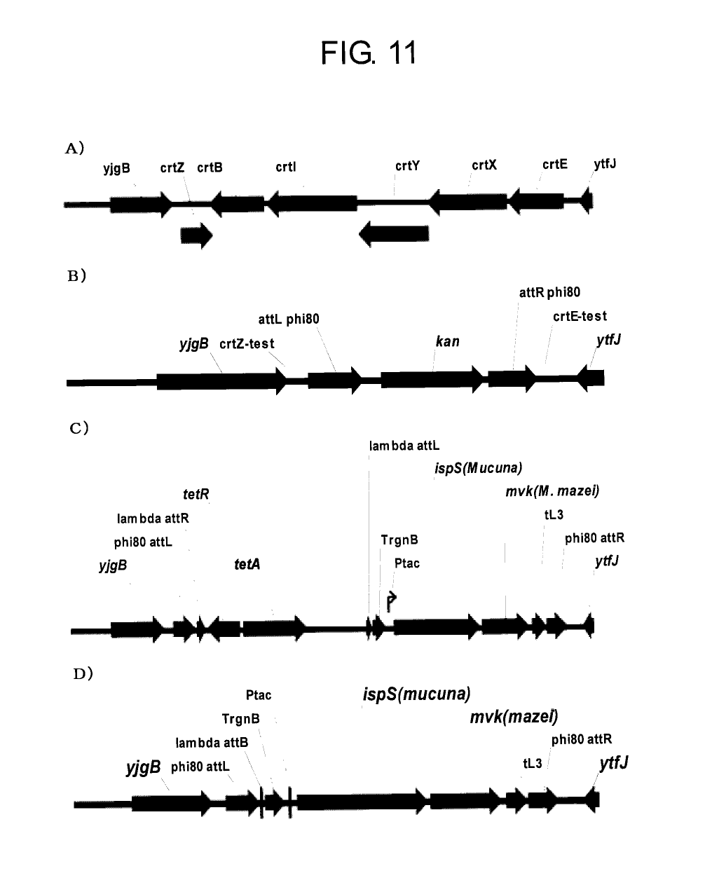

A P. ananatis SC17(0) derivative possessing an attB site of phi80 phage that substituted an ampC gene, an ampH gene or a crt operon was constructed (annotated complete genome sequence of P. ananatis AJ13355 is available as PRJDA162073 or GenBank accession number AP01232.1 and AP012033.1). In order to obtain these strains, kited dependent integration of a PCR-amplified DNA fragment possessing attLphi80-kan-attRphi80 flanking to a 40 bp region homologous to a target site in the genome was carried out according to the previously reported technique (Katashkina J I et al., BMC Mol Biol. 2009; 10:34, which is incorporated herein by reference in its entirety). After the electroporation, cells were cultured on L-agar containing 50 mg/L of kanamycin. DNA fragments used for the substitution of the ampC gene and the ampH gene and the crt operon with attLphi80-kan-attRphi80 were amplified by the reaction using oligonucleotides 1 and 2, 3 and 4, and 5 and 6 (Table 20), respectively. A plasmid PMWattphi (Minaeva N I et al., BMC Biotechnol. 2008; 8:63, which is incorporated herein by reference in its entirety) was used as the template in these reactions. The resulting integrants were designated as SC17(0)ΔampC::attLphi80-kan-attRphi80, SC17(0)ΔampH::attLphi80-kan-attRphi80, and SC17(0)Δcrt::attLphi80-kan-attRphi80. Oligonucleotides 7 and 8, 9 and 10, and 11 and 12 (Table 20) were used for PCR validation of SC17(0)ΔampC::attLphi80-kan-attRphi80, SC17(0)ΔampH::attLphi80-kan-attRphi80, and SC17(0)Δcrt::attLphi80-kan-attRphi80, respectively. Maps of the resulting modified genomes ΔampC::attLphi80-kan-attRphi80, ΔampH::attLphi80-kan-attRphi80 and Δcrt:attLphi80-kan-attRphi80 are shown in FIG. 9A, FIG. 10A, and FIG. 11A, respectively.

Curing of the constructed strains from a kanamycin resistant marker was carried out using a helper plasmid pAH129-cat according to the previously reported technique (Andreeva I G et al., FEMS Microbiol Lett. 2011; 318(1):55-60, which is incorporated herein by reference in its entirety). Oligonucleotides 7 and 8, 9 and 10, and 11 and 12 (Table 20) were used for PCR validation of the resulting strains SC17(0)ΔampC::attBphi80, C17(0)ΔampH::attBphi80, and SC17(0)Δcrt::attBphi80, respectively.

### (5-7) Construction of ISP3-S Strain

The plasmid pAH162-KKDyI-ispS(K) described in (5-5) was integrated in SC17(0)ΔampC::attBphi80 described in (5-6) using a helper plasmid pAH123-cat according to the previously reported technique (Andreeva I G et al., FEMS Microbiol Lett. 2011; 318(1):55-60, which is incorporated herein by reference in its entirety). An oligonucleotide pair 13-7 and 14-8 (Table 20) were used for PCR validation of the resulting integrant. The resulting SC17(0)ΔampC::pAH162-KKDyI-ispS(K) was cured from a vector portion of pAH162-KKDyI-ispS(K) using a helper plasmid pMWintxis-cat possessing an int gene and a xis gene of λ phage according to the previously reported technique (Katashkina J I et al., BMC Mol Biol. 2009; 10:34, which is incorporated herein by reference in its entirety). As a result, a strain SC17(0)ΔampC:: KKDyI-ispS(K) was obtained. Oligonucleotides 7 and 15 (Table 20) were used for PCR validation of the kanamycin sensitive derivative. The construction of SC17(0)ΔampC:: KKDyI-ispS(K) is shown in FIG. 9.

Genomic DNA isolated from the above strain SC17(0)ΔampH::attLphi80-kan-attRphi80 using GeneElute Bacterial genome DNA Kit (Sigma) was electroporated into SC17(0)ΔampH::KKDyI-ispS(K) according to the method of chromosome electroporation previously reported (Katashkina J I et al., BMC Mol Biol. 2009; 10:34, which is incorporated herein by reference in its entirety). Transfer of the mutation ΔampH::attLphi80-kan-attRphi80 was confirmed by PCR using the primers 9 and 10.

The final strain was cured from the kanamycin resistant marker using phi80 Int/Xis dependent technique (Andreeva I G et al., FEMS Microbiol Lett. 2011; 318(1):55-60, which is incorporated herein by reference in its entirety). The modified genome ΔampH::KKDyI-ispS(K) was verified in obtained KmS integrants by PCR using the primers 7 and 15, and subsequently a strain SC17(0)ΔampC::KKDyI-ispS(K) ΔampH::attBphi80 was selected.

The above plasmid pAH162-Para-mvaES was integrated into SC17(0)ΔampC::KKDyhispS(K) ΔampH::attBphi80 using the helper plasmid pAH123-cat (Andreeva I G et al., FEMS Microbiol Lett. 2011; 318(1):55-60, which is incorporated herein by reference in its entirety). Oligonucleotides 13 and 9 and oligonucleotides 14 and 10 (Table 20) were used for validation of the resulting integrant by PCR. This integrant was cured from a vector portion of pAH162-Para-mvaES using phage λ Int/Xis-dependent technique (Katashkina J I et al., BMC Mol Biol. 2009; 10:34, which is incorporated herein by reference in its entirety). Removal of the vector from the chromosome was confirmed by PCR using the primers 9 and 16. As a result, SC17(0)ΔampC::KKDyI-ispS(K)ΔampH::Para-mvaES containing no marker was obtained. A construct of the modified chromosome ΔampH::Para-mvaES is shown in FIG. 10.

The plasmid pAH162-Ptac-ispS(K)-mvk(Mma) described in (5-5) was integrated into genome of SC17(0)Δcrt::attBphi80 using the protocol described in the previous report (Andreeva I G et al., FEMS Microbiol Lett. 2011; 318(1): 55-60, which is incorporated herein by reference in its entirety). The integration of the plasmid was confirmed by the polymerase chain reaction using the primers 11-13 and 12-14.

The modified chromosome SC17(0)Δcrt::pAH162-Ptac-ispS(K)-mvk(Mma) thus constructed was transferred into strain SC17(0)ΔampC::KKDyI-ispS(K)ΔampH::Para-mvaES using the electroporation by genomic DNA in the previous report (Katashkina J I et al., BMC Mol Biol. 2009; 10:34, which is incorporated herein by reference in its entirety). The resulting integrant was cured from a vector portion of pAH162-Ptac-ispS(K)-mvk(Mma) using phage λ Int/Xis-dependent technique (Katashkina J I et al., BMC Mol Biol. 2009; 10:34, which is incorporated herein by reference in its entirety). The structure of a final construct Δcrt::Ptac-ispS(K)-mvk(Mma) (FIG. 11) was confirmed by PCR using the primers 11 and 17.

An integrative expression cassette introduced into this final strain was verified by PCR, and it was found that some of a 5′ portion of KKDyI operon comprising the MVK, PMK, MVD and y1dI genes derived from S. cerevisiae were unexpectedly rearranged. In order to repair this cassette, this strain was electroporated with genomic DNA isolated from strain SC17(0)ΔampC::pAH162-KKDyI-ispS(K) using GeneElute Bacterial Genome DNA Kit (Sigma) according to the technique in the previous report (Katashkina J I et al., BMC Mol Biol. 2009; 10:34, which is incorporated herein by reference in its entirety). The resulting strain contained all genes required for the production of isoprene.

After curing from a vector portion of pAH162-KKDyI-ispS(K) (see the above) using phage λ Int/Xis dependency, strain ISP3-S(P. ananatis SC17(0) ΔampC::attLphi80-KKDyI-ispS(K)-attRphi80 ΔampH::attLphi80-Para-mvaES-attRphi80 Δcrt::attLphi80-Ptac-ispS(K)-mvk(Mma)-attRphi80) containing no marker was obtained.

### (5-8) Construction of Integrative Plasmid Possessing Different Mevalonate Kinase Gene

A KpnI-BamHI fragment of pMW-Ptac-Mclmvk-Ttrp was subcloned into a recognition site for KpnI-Ecl136II in the integrative vector pAH162-λattL-TcR-λattR. As a result, a plasmid pAH162-Ptac-Mclmvk possessing the MVK gene derived from M. concilii and under the control of the tac promoter was constructed.

### (5-9) Construction of AG10265 Strain

The plasmid pAH162-Ptac-Mclmvk described in (5-8) was integrated into the genome of SC17(0)Δcrt::attBphi80 using the helper plasmid pAH123-cat according to the protocol in the previous report (Andreeva I G et al., FEMS Microbiol Lett. 2011; 318(1): 55-60, which is incorporated herein by reference in its entiety).

The modified chromosome Δcrt::pAH123-Ptac-mvk (M. paludicola) was transferred into the strain ISP3-S(5-7) via the electroporation of genomic DNA isolated from Δcrt::pAH162-Ptac-Mclmvk. The resulting integrant was designated as AG10265 (P. ananatis SC17(0) ΔampC::attLphi80-KKIDyI-ispS(K)-attRphi80 ΔampH::attLphi80-Para-mvaES-attRphi80 Δcrt::attLphi80-λattL-TcR-λattR-Ptac-Mclmvk-attRphi80).

TABLE 20
Oligonucleotides used as primers

| | | |
|---|---|---|
| 1 | DampC-phL | 5′-CTGATGAACTGTCACCTGAATGAGTGCT |
| | | GATGAAAATATAGAAAGGTCATTTTTCCTGA |
| | | ATATGCTCA-3′ (SEQ ID NO: 109) |
| 2 | DampC-phR | 5′-ATTCGCCAGCATAACGATGCCGCTGTTG |
| | | AGCTGAGGAACACGTTTGTTGACAGCTGGTC |
| | | CAATG-3′ (SEQ ID NO: 110) |
| 3 | ampH-attL- | 5′-ATGCGCACTCCTTACGTACTGGCTCTAC |
| | phi80 | TGGTTTCTTTGCGAAAGGTCATTTTTCCTGA |
| | | ATATGCTCACA-3′ (SEQ ID NO: 111) |
| 4 | ampH-attR- | 5′-TTAAGGAATCGCCTGGACCATCATCGGC |
| | phi80 | GAGCCGTTCTGACGTTTGTTGACAGCTGGTC |
| | | CAATG-3′ (SEQ ID NO: 112) |
| 5 | crtZ- | 5′-ATGTTGTGGATTTGGAATGCCCTGATCG |
| | attLphi80 | TTTTCGTTACCGGAAAGGTCATTTTTCCTGA |
| | | ATATGCTCA-3′ (SEQ ID NO: 113) |
| 6 | crtE- | 5′-ATGACGGTCTGCGCAAAAAAACACGTTC |
| | attRphi80 | ATCTCACTCGCGCGTTTGTTGACAGCTGGTC |
| | | CAATG-3′ (SEQ ID NO: 114) |
| 7 | ampC-t1 | 5′-GATTCCCACTTCACCGAGCCG-3′ |
| | | (SEQ ID NO: 115) |
| 8 | ampC-t2 | 5′-GGCAGGTATGGTGCTCTGACG-3′ |
| | | (SEQ ID NO: 116) |
| 9 | ampH-t1 | 5′-GCGAAGCCCTCTCCGTTG-3′ |
| | | (SEQ ID NO: 117) |
| 10 | ampH-t2 | 5′-AGCCAGTCAGCCTCATCAGCG-3′ |
| | | (SEQ ID NO: 118) |
| 11 | crtZ-test | 5′-CCGTGTGGTTCTGAAAGCCGA-3′ |
| | | (SEQ ID NO: 119) |
| 12 | crtE-test | 5′-CGTTGCCGTAAATGTATCCGT-3′ |
| | | (SEQ ID NO: 120) |
| 13 | ag-phL-test | 5′-GGATGTAAACCATAACACTCTGCGAA |
| | | C-3′ (SEQ ID NO: 121) |
| 14 | ag-phR-test | 5′-GATTGGTGGTTGAATTGTCCGTAAC-3′ |
| | | (SEQ ID NO: 122) |
| 15 | KKDyI-s-3′ | 5′-TGGAAGGATTCGGATAGTTGAG-3′ |
| | | (SEQ ID NO: 123) |

### Example 6

### Preparation of Pantoea Strain Having Improved Expression of Ppa Gene and Production of Isoprene by Cultivation Using the Same

A strain where a promoter inherent to an endogenous ppa gene (pyrophosphate phosphatase gene) was substituted with another strong promoter to augment the expression of the endogenous ppa gene in P ananatis strain was made by the following procedure.

### (6-1) Enhancement of Ppa Gene Expression in AG10265 Strain

### (6-1-1) Preparation of Strain Having Substituted Ppa Gene Promoter in Wild Type SC17(0)

There are two genes encoding pyrophosphate phosphatase, PAJ_2344(ppa-1) and PAJ_2736(ppa-2) in P. ananatis. In order to enhance the expression of the PAJ_2344(ppa-1) gene and the PAJ_2736(ppa-2) gene, a strain where a promoter sequence and an SD sequence of the PAJ_2344 (ppa-1) gene and the PAJ_2736 (ppa-2) gene were substituted with Ptac and φ10, respectively in P. ananatis SC17(0) was prepared by the λRed method. The λRed method was carried out according to the method described in BMC Mol Biol. 2009; 10:34, which is incorporated herein by reference in its entirety. Annotated genome sequence information for P. ananatis AJ13355 is available as GenBank accession numbers AP012032.1 and AP012033.1.

Genomic DNA was extracted from P. ananatis SC17(0) strain Ptac-lacZ (RU application 2006134574, WO2008/090770, US2010062496, all of which are incorporated herein by reference in their entireties), and used as the template for PCR. λattL-Km^{r}-λattR-Ptac where the Ptac promoter was ligated downstream of λattL-Km^{r}-λattR has been integrated upstream of the lacZ gene in P. ananatis SC17(0) strain Ptac-lacZ (see WO2011/87139 A1, which is incorporated herein by reference in its entirety).

PCR was carried out using synthesized oligonucleotides consisting of the nucleotide sequences of PAJ_2344-F and PAJ_2344-R as the primers in order to substitute the promoter region of PAJ_2344 (ppa-1) gene, using synthesized oligonucleotides consisting of the nucleotide sequences of PAJ_2736-F and PAJ_2736-R as the primers in order to substitute the promoter region of PAJ_2736 (ppa-2) gene, and using PrimeStar MAX DNA polymerase (supplied from TaKaRa Bio). A reaction solution was prepared according to the composition attached to the kit, and the reaction of a cycle of 98° C. for 10 seconds, 55° C. for 5 seconds, 72° C. for 20 seconds was repeated 30 times. As a result, a fragment comprising λattL-Km^{r}-λattR-φ10 for substituting the promoter was obtained.

A plasmid RSF-Red-TER (U.S. Pat. No. 7,919,284B2) was introduced into SC17(0) by the electroporation according to the standard method. The resulting strain was designated as SC17(0)/RSF-Red-TER. Competent cells of SC17(0)/RSF-Red-TER were prepared and then the purified fragment comprising λattL-Km^{r}-λattR-φ10 for substituting the promoter was introduced thereto by the electroporation. After the electroporation, a colony that had acquired kanamycin resistance was obtained.

Next, by PCR using two synthesized DNA primers represented by ppa_p1-R (Table 21) and ppa_p1-F (Table 21) or ppa_p2-R (Table 21) and ppa_p2-F (Table 21), it was confirmed that a sequence derived from a PCR fragment had been inserted into an upstream region of PAJ_2344 (ppa-1) gene or an upstream region of PAJ_2736 (ppa-2) gene. Bacterial strains where the insertion of the fragment had been confirmed were obtained. The bacterial strains thus obtained were designated as P. ananatis SC17(0)Ptac-φ10-ppa1 and P. ananatis SC17(0)Ptac-φ10-ppa2.

(6-1-2) Preparation of AG10265 Strain with Ppa Gene Having Substituted Promoter

Genomic DNA was extracted from SC17(0)Ptac-φ10-ppa1 using PurElute Bacterial Genomic kit (EdgeBio). Using this as the template, a fragment from 1 kb upstream of to 1 kb downstream of the PAJ_2344 (ppa-1) gene was amplified by PCR using synthesized nucleotides consisting of the nucleotide sequences of ppa-1-g1000-F and ppa-1-g1000-R primer (Table 21) as the primers. Also, using genomic DNA extracted from SC17(0)Ptac-φ10-ppa2 as the template, a fragment from 1 kb upstream of to 1 kb downstream of the PAJ_2736 (ppa-2) gene was amplified by PCR using synthesized nucleotides consisting of the nucleotide sequences of ppa-2-g1000-F and ppa-2-g1000-R primer (Table 21) as the primers. PCR was carried out using PrimeStar GXL DNA polymerase (supplied from TaKaRa Bio), a reaction solution was prepared according to the composition attached to the kit, and the reaction of a cycle of 98° C. for 10 seconds, 60° C. for 15 seconds, 68° C. for 5 minutes was repeated 40 times. As a result, the fragment comprising from 1 kb upstream of to 1 kb downstream of the PAJ_2344 (ppa-1) gene or the PAJ_2736 (ppa-2) gene was obtained. The resulting fragment was purified, 600 ng of the PCR product was introduced into AG10265 by electroporation to perform transformation, and colonies that had acquired the kanamycin resistance were obtained.

By colony PCR using synthesized nucleotides consisting of the nucleotide sequences of Ppa_p1-F and Ppa_p1-R in Table 21 as the primers, it was confirmed that the promoter of the PAJ_2344 (ppa-1) gene was substituted in the resulting transformant. Likewise, by colony PCR using synthesized nucleotides consisting of the nucleotide sequences of Ppa_p2-F and Ppa_p2-R in Table 21 as the primers, it was confirmed that the promoter of the PAJ_2736 (ppa-2) gene was substituted. In these strains, the promoter region of the PAJ_2344 (ppa-1) gene or the PAJ_2736 (ppa-2) gene has been substituted with Ptac-φ10. Of these, each one clone was selected, and designated as AG10265 Ptac-φ10-ppa1, or AG10265 Ptac-φ10-ppa2.

TABLE 21
List of primers used

| Primer name | Sequence (5′-) |
|---|---|
| Ppa_p1-F | TTACTGACCTGTTGATCGGC |
| | (SEQ ID NO: 124) |
| Ppa_p1-R | CTGAGGATGTTTTCCGCCTG |
| | (SEQ ID NO: 125) |
| PAJ_2344-F | CTGCTGCCGCCTCTCCTGCACGACATCAGC |
| | TGAACTCTCCTCCTCTGTCTGACGTCATCC |
| | TGAAGCCTGCTTTTTTATACTAAGTTGGC |
| | (SEQ ID NO: 126) |
| PAJ_2344-R | AACGGCGGCAGAGGCGGTGATAAAAAAGAG |
| | ACAAGCAAAAAGCGGTTTTACCAGGTTCAT |
| | TATATCTCCTTCTTAAAGTTAAACAAAATT |
| | (SEQ ID NO: 127) |
| ppa-1-g1000-F | TGGGGCACACGCGTTTATGAGC |
| | (SEQ ID NO: 128) |
| ppa-1-g1000-R | GTAAGCGCCCGGTCCGACCTTA |
| | (SEQ ID NO: 129) |
| Ppa_p2-F | TGCTGCGGGCATTGTTTCCC |
| | (SEQ ID NO: 130) |
| Ppa_p2-R | TCGGCATTGGCCGGGATCTC |
| | (SEQ ID NO: 131) |
| PAJ_2736-F | TGTTCATCACGGCCAAATAGCGTTGAGGAG |
| | CATTCCGAAACGCCAAAAAGGGGGCGTACT |
| | TGAAGCCTGCTTTTTTATACTAAGTTGGC |
| | (SEQ ID NO: 132) |
| PAJ_2736-R | AATAATTACGTAGATATCTTCTGGCAGGTC |
| | TTTACCTGCTGGCACCTGGTTCAAACTCAT |
| | TATATCTCCTTCTTAAAGTTAAACAAAATT |
| | (SEQ ID NO: 133) |
| ppa-2-g1000-F | CATGAAAAGAGGATGGGTGTTG |
| | (SEQ ID NO: 134) |
| ppa-2-g1000-R | GTTATCTACCTTAAGATCCTGT |
| | (SEQ ID NO: 135) |

### (6-1-3) Confirmation of Amount of Expressed Pyrophosphate Phosphatase

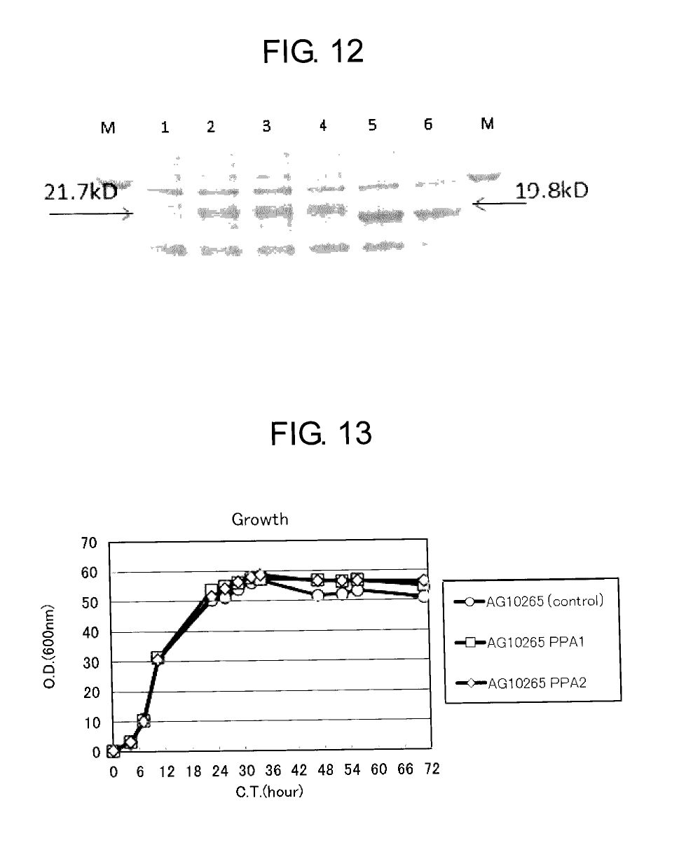

An amount of an expressed protein of pyrophosphate phosphatase (PPA) in the AG10265 Ptac-φ10-ppa1 strain and the AG10265 Ptac-φ10-ppa2 strain was confirmed by SDS-PAGE. Cells of the AG10265 strain, the AG10265 Ptac-φ10-ppa1 strain and AG10265 Ptac-φ10-ppa2 strain were cultured with shaking in 3 mL of LB medium containing 50 mg/L of kanamycin at 30° C. overnight. The microbial cells were collected, then washed twice with ice-cooled 50 mM Tris buffer (Tris-HCl, pH 8.0), and disrupted using a multibead shocker (Yasui Kikai, Japan, 4° C., 5 cycles of ON for 60 seconds and OFF for 60 seconds, 2500 rpm). A solution of the disrupted microbial cells was centrifuged at 14,000 rpm for 20 minutes to remove cell debris. The resulting supernatant fraction was handled as a soluble protein fraction. The soluble protein fraction was quantified by the BCA method, and 5 μg of the soluble protein was electrophoresed on SDS-PAGE (supplied from Invitrogen, NuPAGE: SDS-PAGE Gel System). Subsequently, CBB staining and decoloration were carried out according to the standard methods. A photograph of a gel around a mass of the PPA protein was shown in FIG. 12. As a result, increase of the expression amount of the protein each presumed to be PPA was confirmed in the AG10265 Ptac-φ10-ppa1 strain and the AG10265 Ptac-q 10-ppa2 strain. From density of bands on SDS-PAGE after the electrophoresis, the expression amount of the protein each presumed to be PPA in the AG10265 Ptac-φ10-ppa1 strain and the AG10265 Ptac-φ10-ppa2 strain was estimated to be about 1.5 to 2.0 folds higher than that in the original strain (AG10265).

### (6-1-4) Introduction of Plasmid for Expression of Isoprene Synthase

Electrocells of the AG10265 strain, the AG10265 Ptac-φ10-ppa1 strain and the AG10265 Ptac-φ10-ppa2 strain were prepared, and pSTV28-Ptac-IspSM (see Reference Example 3-5) was introduced thereto by the electroporation. The resulting isoprene-producing bacterial strains were designated as AG10265 (Control), AG10265 PPA1 and AG10265 PPA2, respectively.

(6-2) Conditions for Jar Cultivation of P. ananatis Isoprene-Producing Bacteria

A one liter volume fermenter was used for the cultivation of P. ananatis isoprene-producing bacteria. Glucose medium was prepared as the composition shown in Table 22. Microbial cells of the isoprene-producing bacterial strain were applied onto an LB plate containing 60 mg/L of chloramphenicol and cultured at 34° C. for 16 hours. 0.3 L of the glucose medium was placed in the 1 L volume fermenter, and microbial cells sufficiently grown on one plate were inoculated thereto to start the cultivation. A culture condition was pH 7.0 (controlled with ammonia gas), 30° C., ventilation of 150 mL/minute, and stirring such that an oxygen concentration in the medium was 5% or higher. During the cultivation, a glucose solution adjusted to 500 g/L was continuously added to the medium such that a glucose concentration in the medium was 10 g/L or higher. Finally, AG10265 (Control), AG10265 PPA1 and AG10265 PPA2 consumed 92.2 g, 95.8 g and 85.7 g of glucose in the cultivation for 71 hours.

TABLE 22
Composition of glucose medium

| | (Final concentration) | |
|---|---|---|
| Group A | | |
| Glucose | 80 | g/L |
| MgSO₄•7aq | 2.0 | g/L |
| Group B | | |
| (NH₄)₂SO₄ | 2.0 | g/L |
| KH₂PO₄ | 2.0 | g/L |
| FeSO₄•7aq | 20 | mg/L |
| MnSO₄•5aq | 20 | mg/L |
| Yeast Extract | 4.0 | g/L |

After preparing 0.15 L of Group A and 0.15 L of Group B, they were heated and sterilized at 115° C. for 10 minutes. After cooling, Group A and Group B were mixed, and chloramphenicol (60 mg/L) was added thereto to use as the medium.

### (6-3) Method of Inducing to Isoprene-Producing Phase

Since P. ananatis isoprene-producing bacterial strain expresses a gene upstream of the mevalonate pathway with an arabinose-inducible promoter, a production amount of isoprene is remarkably improved in the presence of L-arabinose (Wako Pure Chemical Industries). For inducing to an isoprene-producing phase, broth in the fermenter was analyzed with time, and when absorbance at 600 nm became 16, L-arabinose at a final concentration of 20 mM was added.

### (6-4) Method of Measuring Isoprene Gas Concentration in Fermentation Gas

From immediately after starting the cultivation, evolved gas was collected in a 1 L gas bag with time, and a concentration of the isoprene gas contained in the evolved gas was measured using gas chromatography based on the condition described in Reference Example 4-3.

### (6-5) Amount of Isoprene Formed in Jar Cultivation

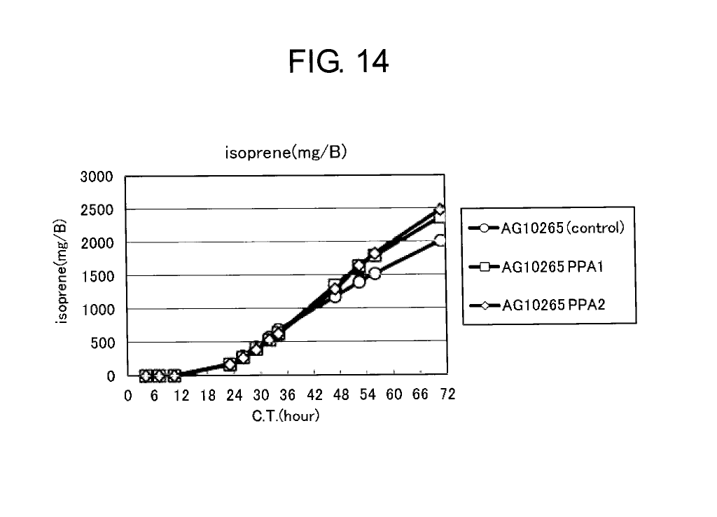

Microbial cells of AG10265 (control), AG10265 PPA1 and AG10265 PPA2 were cultured under the above jar cultivation condition, and an amount of formed isoprene was measured. Profiles of growth of the microbial cells and amounts of isoprene produced until 71 hours after starting the cultivation are shown in FIG. 13 and FIG. 14, respectively. The descending order of the production amount of total isoprene was AG10265 PPA2, AG10265 PPA1, then AG10265 Control (FIG. 3). The amounts of total isoprene produced by AG10265 PPA2, AG10265 PPA1, and AG10265 (Control) were 2478 mg, 2365 mg and 2013 mg, respectively. This has indicated that the P. ananatis isoprene-producing bacterial strain where the expression amount of pyrophosphate phosphatase was enhanced exhibited the more excellent ability to produce isoprene than P. ananatis isoprene-producing bacterial strain where the expression amount of pyrophosphate phosphatase was not enhanced.

### Reference Example 8

### Preparation of SWITCH-PphosC-1(S) Strain

### 1. Preparation of Plasmid for Upstream of MVA Pathway for Chromosome Fixation

A plasmid for upstream of MVA pathway for chromosome fixation, pAH162-PphoC-mvaES was constructed as follows.

(1-1) Construction of Arabinose-Inducible Plasmid for Expressing Mevalonate Pathway Upstream Gene (mvaES [Enterococcus faecalis (E. faecalis)]) Derived from E. faecalis, pMW-Para-mvaES-Ttrp
(1-1-1) Chemical Synthesis and Cloning of mvaES (E. faecalis) Gene

A nucleotide sequence (GenBank/EMBL/DDBJ accession ID AF290092.1) and an amino acid sequence (mvaS, GenPept accession ID AAG02438.1, mvaE, GenPept accession ID AAG02439.1) of the mvaES gene encoding the mevalonate pathway upstream gene (mvaES [Enterococcus faecalis (E. faecalis)]) derived from E. faecalis are known publicly (see Wilding, E I et al., J. Bacteriol. 182 (15), 4319-4327 (2000), which is incorporated herein by reference in its entirety). Based on this information, a mvaE gene and a mvaS gene in which their codon usages had been optimized for E. coli were designed and designated as EFmvaE and EF mvaS, respectively. Nucleotide sequences of EFmvaE and EFmvaS are shown in SEQ ID NO:139 and SEQ ID NO:140, respectively. DNA sequences of EFmvaE and EFmvaS prepared by chemical synthesis were cloned into a plasmid for expression pUC57 (supplied from GenScript), and designated as pUC57-EFmvaE and pUC57-EFmvaS, respectively. Nucleotide sequences of pUC57-EFmvaE and pUC57-EFmvaS are shown in SEQ ID NO:141 and SEQ ID NO:142, respectively.

(1-1-2) Construction of Plasmid pMW-P_{trc}-mvaES-Tₜᵣₚ Used for in-Fusion Method

A plasmid pMW-P_{trc}-mvaES-Tₜᵣₚ used for the In-fusion method was constructed according to the following procedure.

A plasmid pMW219 (supplied from Nippon Gene, Part number: 310-02571) was digested with SmaI, and this digested plasmid was purified. The resulting plasmid was designated as pMW219/SmaI.

In order to obtain a gene of a trc promoter (P_{trc}) region, PCR was carried out using a plasmid pTrcHis2B having the P_{trc} region as the template and using synthesized oligonucleotides consisting of the nucleotide sequences of SEQ ID NO:143 and SEQ ID NO:144 as primers.

In order to obtain a mvaE gene portion, PCR was carried out using the plasmid pUC57-EFmvaE as the template and using synthesized oligonucleotides consisting of the nucleotide sequences of SEQ ID NO:145 and SEQ ID NO:146 as the primers.

In order to obtain a mvaS gene portion, PCR was carried out using the plasmid pUC57-EFmvaS as the template and using synthesized oligonucleotides consisting of the nucleotide sequences of SEQ ID NO:146 and SEQ ID NO:147 as the primers.

In order to obtain a gene of a trp terminator (Tₜᵣₚ) region, PCR was carried out using a plasmid pSTV-P_{tac}-Tₜᵣₚ having the Tₜᵣₚ region as the template and using synthesized oligonucleotides consisting of the nucleotide sequences of SEQ ID NO:148 and SEQ ID NO:149 as the primers.

In the above four PCR cases, PrimeStar polymerase (supplied from TaKaRa Bio) was used as the enzyme, a reaction solution was prepared according to instructions provided by the supplier of the enzyme, and the reaction of a cycle of 98° C. for 10 seconds, 55° C. for 5 seconds and 72° C. for 60 seconds/kb was repeated 30 times. As a result, the PCR product comprising the gene of the P_{trc} region, the mvaE gene, the mvaS gene and the gene of the Tₜᵣₚ region was obtained.

(1-1-3) Construction of Plasmid pMW-P_{trc}-mvaES-Tₜᵣₚ Used for in-Fusion Method

Subsequently, PCR was carried out using the purified PCR product comprising P_{trc} and the purified PCR product comprising the mvaE gene as the template and using synthesized oligonucleotides consisting of SEQ ID NO:143 and SEQ ID NO:146 as the primers. Also PCR was carried out using the purified PCR product comprising the mvaS gene and the purified PCR product comprising Tₜᵣₚ as the template and using synthesized oligonucleotides consisting of SEQ ID NO:147 and SEQ ID NO:150 as the primers. As a result, the PCR product comprising the gene of the P_{trc} region and the mvaE gene, and the PCR product comprising the mvaS gene and the gene of the Tₜᵣₚ region were obtained.

Subsequently, the PCR product comprising the gene of the P_{trc} region and the mvaE gene, the PCR product comprising the mvaS gene and the gene of the Tₜᵣₚ region, and the plasmid pMW219/SmaI digested above were ligated using In-Fusion HD Cloning Kit (supplied from Clontech). The resulting plasmid was designated as pMW-P_{trc}-mvaES-Tₜᵣₚ. A sequence of obtained pMW-P_{trc}-mvaES-Tₜᵣₚ, is shown in SEQ ID NO:151.

(1-1-4) Construction of Plasmid pMW-Pₐᵣₐ-mvaES-Ttrp

The arabinose-inducible plasmid for expression of a mevalonate pathway upstream gene, pMW-Pₐᵣₐ-mvaES-Ttrp was constructed according to the following procedure.

PCR was carried out using the plasmid pMW-P_{trc}-mvaES-Tₜᵣₚ prepared in (1-1-3) as the template and using synthesized oligonucleotides consisting of the nucleotide sequences of SEQ ID NO:152 and SEQ ID NO:153 as the primers.

PCR was carried out using a plasmid pKD46 (see Proc. Natl. Acad. Sci. USA, 2000, vol. 97, No. 12, p 6640-6645, which is incorporated herein by reference in its entirety) comprising a gene of a P_{araC} region, an araC gene and a gene of a P_{araBAD} region (hereinafter also collectively referred to as a “gene of a Pₐᵣₐ region”) as the template and using synthesized oligonucleotides consisting of the nucleotide sequences of SEQ ID NO:154 and SEQ ID NO:155 as the primers.

As a result, the PCR product comprising the plasmid pMW and the mvaES gene and the PCR product comprising the gene of the Para region were obtained. Purified these PCR products were ligated using In-Fusion HD Cloning Kit (supplied from Clontech). The resulting arabinose-inducible plasmid for expression of the mevalonate pathway upstream gene derived from E. faecalis (mvaES (E. faecalis)) was designated as PMW-Pₐᵣₐ-mvaES-Ttrp. A nucleotide sequence of PMW-Pₐᵣₐ-mvaES-Ttrp is shown in SEQ ID NO:156.

### (1-2) Construction of Integrative Conditional Replication Plasmid Possessing Mevalonate Pathway Upstream

A vector pAH162-λattL-TcR-λattR (Minaeva N I et al., BMC Biotechnol. 2008; 8:63, which is incorporated herein by reference in its entirety) was used in order to construct an integrative plasmid possessing an upstream gene and a downstream gene of the mevalonate pathway.

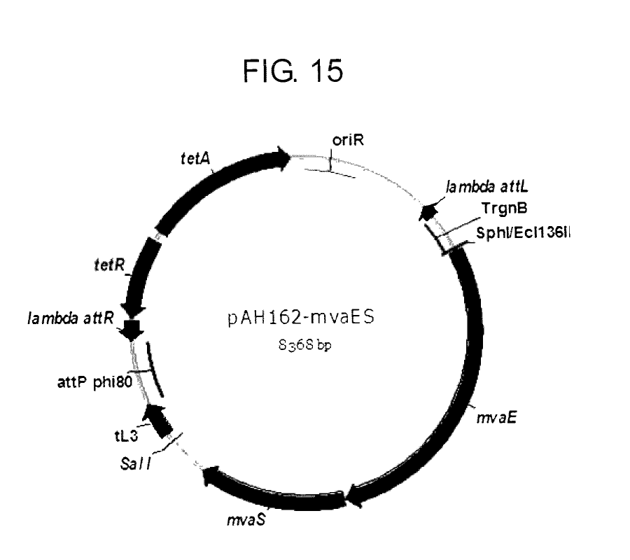

In order to obtain a promoter deletion mutant of an operon, an Ec1136II-SalI fragment of PMW-Para-mvaES-Ttrp was subcloned into the vector pAH162-λattL-TcR-λattR. A map of the resulting plasmid is shown in FIG. 15.

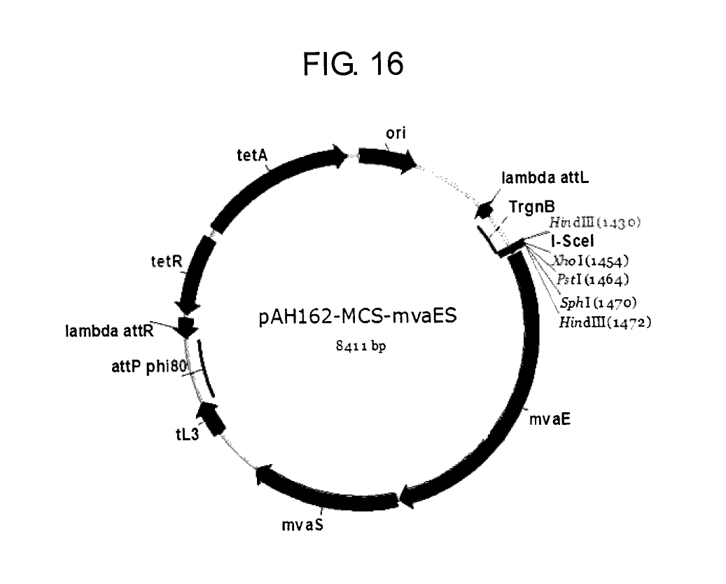

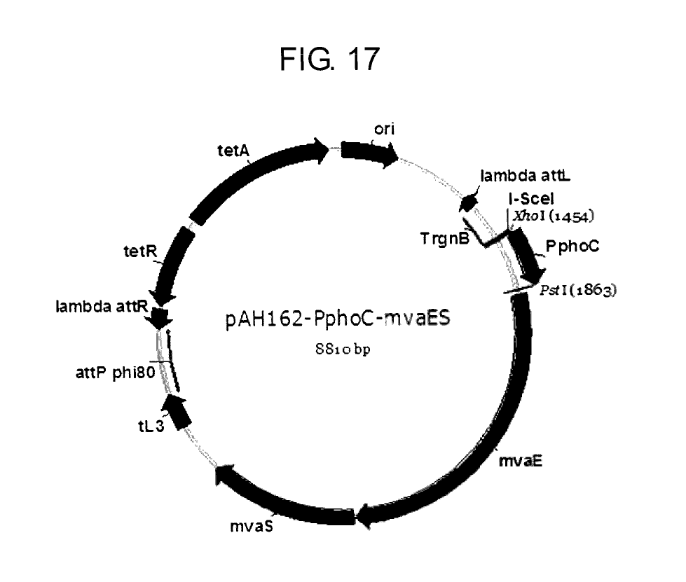

A set of plasmids for chromosome fixation holding the mvaES gene under the control of a different promoter was constructed. For this purpose, a polylinker comprising recognition sites for I-SceI, XhoI, PstI and SphI was inserted into only one recognition site for HindIII located upstream of the mvaES gene. In order to accomplish this purpose, annealing was carried out using synthesized oligonucleotides consisting of the nucleotide sequences of SEQ ID NO:157 and SEQ ID NO:158 and polynucleotide kinase, and the resulting fragment was inserted into a plasmid pAH162-mvaES cleaved with HindIII by a ligation reaction. The resulting plasmid pAH162-MCS-mvaES (FIG. 16) is convenient for cloning a promoter while keeping a desired orientation before the mvaES gene. A DNA fragment holding a regulatory region of a phoC gene was formed by PCR using Genomic DNA from P. ananatis SC17(0) strain (Katashkina J I et al. BMC Mol Biol. 2009; 10:34, which is incorporated herein by reference in its entirety) as the template and using oligonucleotides consisting of the nucleotide sequences of SEQ ID NO:159 and SEQ ID NO:160, and the resulting fragment was cloned into a recognition site for an appropriate restriction enzyme in pAH162-MCS-mvaES. The resulting plasmid is shown in FIG. 17. The cloned promoter fragment was sequenced, and confirmed to precisely correspond to a predicted nucleotide sequence. The resulting plasmid was designated as pAH162-PphoC-mvaES.

### 2. Preparation of Plasmid for MVA Pathway Downstream for Chromosome Fixation

An integrative plasmid pAH162-Km-Ptac-KDyI was constructed as follows, as a plasmid for MVA pathway downstream for chromosome fixation.

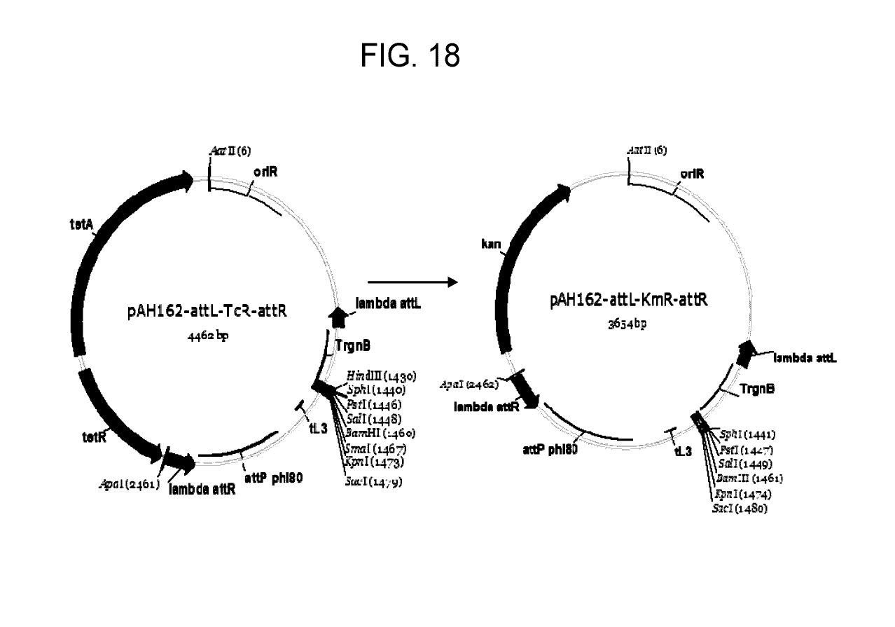

An AatII-ApaI fragment of pAH162-λattL-Tc^{R}-λattR (Minaeva N I et al. BMC Biotechnol. 2008; 8:63, which is incorporated herein by reference in its entirety) comprising a tetAR gene was substituted with a DNA fragment obtained by PCR using synthesized oligonucleotides consisting of the nucleotide sequences of SEQ ID NO:161 (primer 11) and SEQ ID NO:162 (primer 12) as the primers and using a plasmid pUC4K (Taylor L A and Rose R E. Nucleic Acids Res. 16, 358, 1988, which is incorporated herein by reference in its entirety) as the template. As a result, pAH162-λattL-Km^{R}-λattR was obtained (FIG. 18).

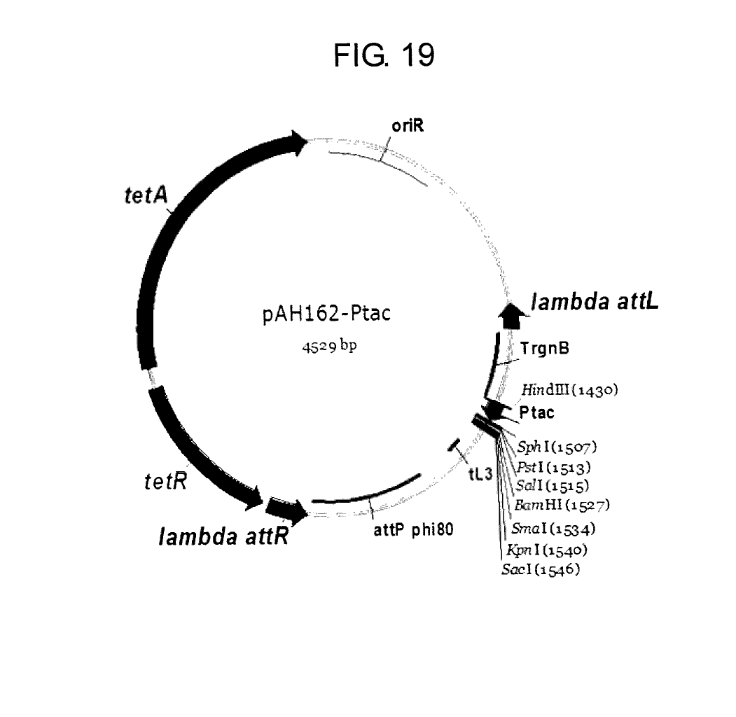

The P_{tac} promoter was inserted into a recognition site for HindIII-SphI in the integrative vector pAH162-λattL-Tc^{R}-λattR (Minaeva N I et al. BMC Biotechnol. 2008; 8:63, which is incorporated herein by reference in its entirety). As a result, an integrative vector pAH162-P_{tac} was constructed. The cloned promoter fragment was sequenced. A map of pAH162-P_{tac} is shown in FIG. 19.

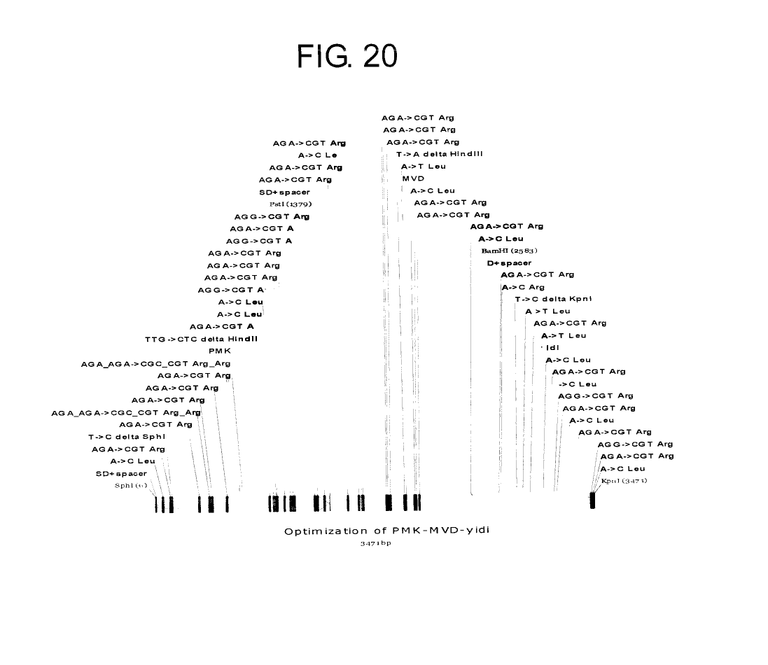

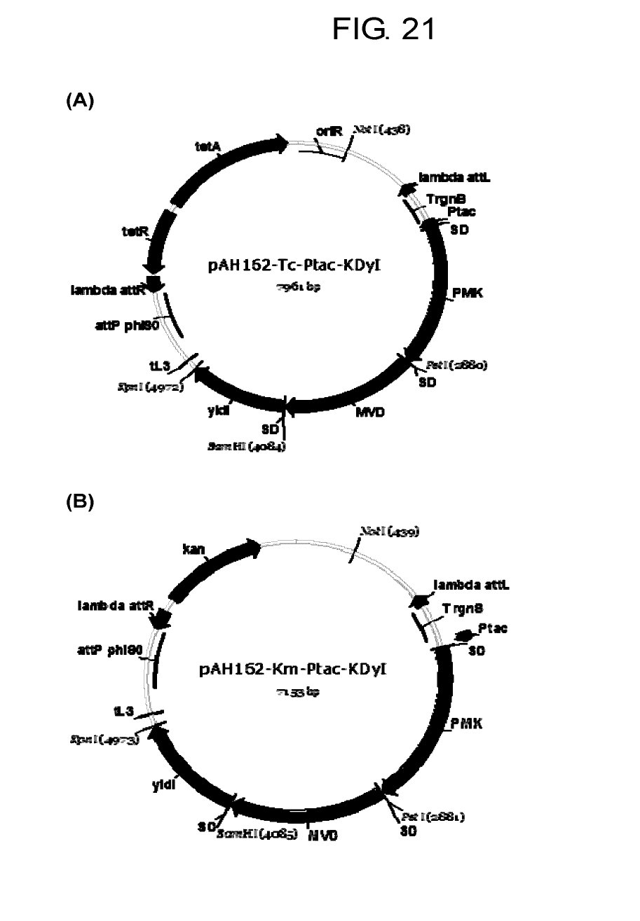

A DNA fragment chemically synthesized by ATG Service Gene (Russia) and holding the PMK gene, the MVD gene and the y1dI gene derived from S. cerevisiae having substituted rare codons (FIG. 20) was subcloned into a recognition site for SphI-KpnI restriction endonuclease in the integrative plasmid pAH162-Ptac. A chemically synthesized DNA sequence comprising a KDyI operon is shown in SEQ ID NO:184. The resulting plasmid pAH162-Tc-Ptac-KDyI holding an expression cassette Ptac-KDyI is shown in FIG. 21A. Subsequently, a NotI-KpnI fragment of pAH162-Tc-Ptac-KDyI holding a tetAR gene was substituted with a corresponding fragment of pAH162-λattL-KmR-λattR. As a result, a plasmid pAH162-Km-Ptac-KDyI having a kanamycin resistant gene kan as a marker was obtained (FIG. 21B).

### 3. Preparation of Plasmid Integrating MVA Gene

A chemically synthesized DNA fragment comprising a coding portion of a presumed mvk gene derived from SANAE (for complete genome sequence, see GenBank Accession Number AP011532) that was Methanocella paludicola and ligated to a classical SD sequence was cloned into a recognition site for PstI-KpnI in the above integrative expression vector pAH162-P_{tac}.

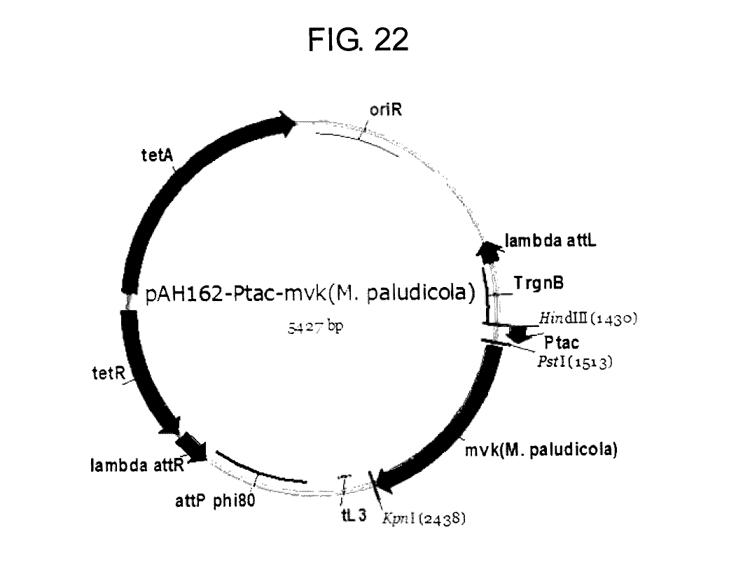

A map of the integrative plasmid holding the mvk gene is shown in FIG. 22.

4. Construction of Recipient Strain SC17(0) ΔampC::attBₚₕᵢ₈₀ ΔampH::attBₚₕᵢ₈₀ Δcrt::P_{tac}-mvk(M. paludicola)

Chromosomal modifications ΔampH::attBₚₕᵢ₈₀ and ΔampC::attBₚₕᵢ₈₀ were introduced into P. ananatis SC17(0) stepwise using two step technique comprising λRed dependent integration of a PCR-amplified DNA fragment comprising the gene kan flanking to attLₚₕᵢ₈₀ and attRₚₕᵢ₈₀ and a 40 bp sequence homologous to a target chromosome site (Katashkina J I et al. BMC Mol Biol. 2009; 10:34, which is incorporated herein by reference in its entirety), followed by phage phi80 Int/Xis dependent cleavage of the kanamycin resistant marker (Andreeva I G et al. FEMS Microbiol Lett. 2011; 318(1):55-60, which is incorporated herein by reference in its entirety). SC17(0) is a λRed resistant derivative of P. ananatis AJ13355 (Katashkina J I et al. BMC Mol Biol. 2009; 10:34, which is incorporated herein by reference in its entirety); an annotated complete genome sequence of P. ananatis AJ13355 is available as PRJDA162073 or GenBank Accession Numbers AP012032.1 and AP012033.1. DNA fragments each used for the integration into ampH and ampC genes, respectively were formed by PCR using a plasmid pMWattphi (Minaeva N I et al. BMC Biotechnol. 2008; 8:63, which is incorporated herein by reference in its entirety) as the template and using synthesized oligonucleotides consisting of the nucleotide sequences of SEQ ID NO:163 (primer 13) and SEQ ID NO:164 (primer 14) and synthesized oligonucleotides consisting of the nucleotide sequences of SEQ ID NO:165 (primer 15) and SEQ ID NO:166 (primer 16) as the primers. The resulting chromosomal modification was verified by PCR using synthesized oligonucleotides consisting of the nucleotide sequences of SEQ ID NO:167 (primer 17) and SEQ ID NO:168 (primer 18) and synthesized oligonucleotides consisting of the nucleotide sequences of SEQ ID NO:169 (primer 19) and SEQ ID NO:170 (primer 20) as the primers.

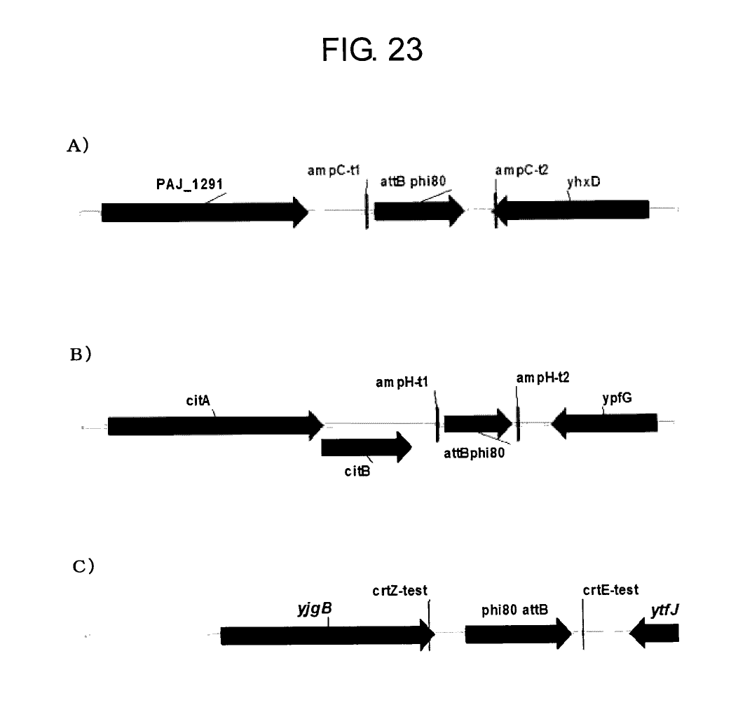

In parallel, a derivative of P. ananatis SC17(0) holding an attB site of the phage phi80 in place of an operon crt (positioned on a megaplasmid pEA320 320 kb that is a portion of the genome of P. ananatis AJ13355) was constructed. In order to obtain this strain, the λRed dependent integration of a PCR-amplified DNA fragment holding attLₚₕᵢ₈₀-kan-attRₚₕᵢ₈₀ flanking to a 40 bp region homologous to a target site in the genome was carried out according to the previously described technique (Katashkina J I et al. BMC Mol Biol. 2009; 10:34, which is incorporated herein by reference in its entirety). A DNA fragment used for substitution of the operon crt with attLₚₕᵢ₈₀-kan-attRₚₕᵢ₈₀ was amplified by PCR using synthesized oligonucleotides consisting of the nucleotide sequences of SEQ ID NO:171 (primer 21) and SEQ ID NO:172 (primer 22). The plasmid pMWattphi (Minaeva N I et al. BMC Biotechnol. 2008; 8:63, which is incorporated herein by reference in its entirety) was used as the template in this reaction. The resulting integrant was designated as SC17(0)Δcrt::attLₚₕᵢ₈₀-kan-attRₚₕᵢ₈₀. A chromosomal structure of SC17(0)Δcrt::attLphi80-kan-attRₚₕᵢ₈₀ was verified by PCR using synthesized oligonucleotides consisting of the nucleotide sequences of SEQ ID NO:173 (primer 23) and SEQ ID NO:174 (primer 24). The kanamycin resistant marker was removed from the constructed strain using the helper plasmid pAH129-cat according to the previously reported technique (Andreeva I G et al. FEMS Microbiol Lett. 2011; 318(1):55-60, which is incorporated herein by reference in its entirety). The resulting strain SC17(0) Δcrt::attBₚₕᵢ₈₀ was verified by PCR using synthesized oligonucleotides consisting of the nucleotide sequences of SEQ ID NO:173 (primer 23) and SEQ ID NO:174 (primer 24). Maps of the resulting modified genomes ΔampC::attBₚₕᵢ₈₀, ΔampH::attBₚₕᵢ₈₀ and Δcrt::attBₚₕᵢ₈₀ are shown in FIGS. 23A, 7B and 7C, respectively.

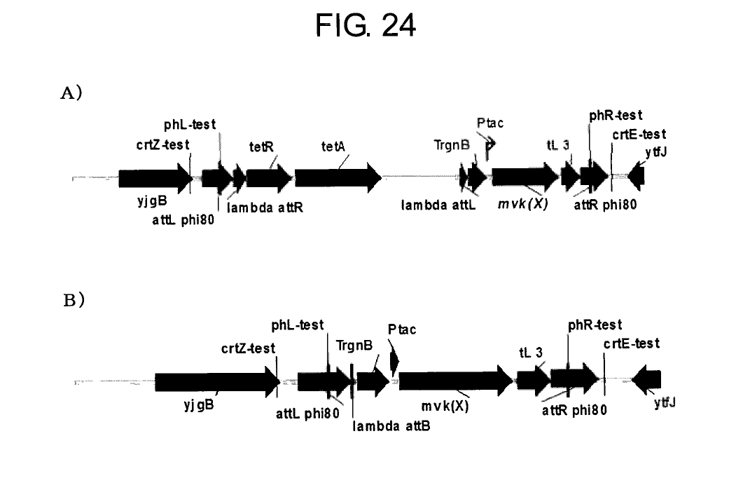

The above plasmid pAH162-Ptac-mvk (M. paludicola) was integrated into genome of SC17(0) Δcrt::attBₚₕᵢ₈₀ according to the previously reported protocol (Andreeva I G et al. FEMS Microbiol Lett. 2011; 318(1):55-60, which is incorporated herein by reference in its entirety). The integration of the plasmid was confirmed by the polymerase chain reaction using synthesized oligonucleotides consisting of the nucleotide sequences of SEQ ID NO:171 (primer 21), SEQ ID NO:173 (primer 23), SEQ ID NO:172 (primer 22) and SEQ ID NO:174 (primer 24). As a result, a strain SC17(0)Δcrt::pAH162-P_{tac}-mvk (M. paludicola) was obtained. A map of the modified genome Δcrt::pAH162-P_{tac}-mvk (M. paludicola) is shown in FIG. 24A.

Subsequently, transfer from SC17(0)Δcrt::pAH162-P_{tac}-mvk (M. paludicola) toSC17(0) ΔampC::attBₚₕᵢ₈₀ ΔampH::attBₚₕᵢ₈₀ was carried out via the electroporation of genomic DNA (Katashkina J I et al. BMC Mol Biol. 2009; 10: 34, which is incorporated herein by reference in its entirety). The resulting strain was cured from a vector portion of the integrative plasmid pAH162-Ptac-mvk (M. paludicola) using the previously reported helper plasmid pMW-intxis-cat (Katashkina J I et al. BMC Mol Biol. 2009; 10: 34, which is incorporated herein by reference in its entirety). As a result, the marker-deleted strain SC17(0) ΔampH::attB_{φ80} ΔampC::attB_{φ80} Δcrt::P_{tac}-mvk (M. paludicola) was obtained. A map of the modified genome Δcrt::P_{tac}-mvk (M. paludicola) is shown in FIG. 24B.

### 5. Construction of Strain SWITCH-PphoC

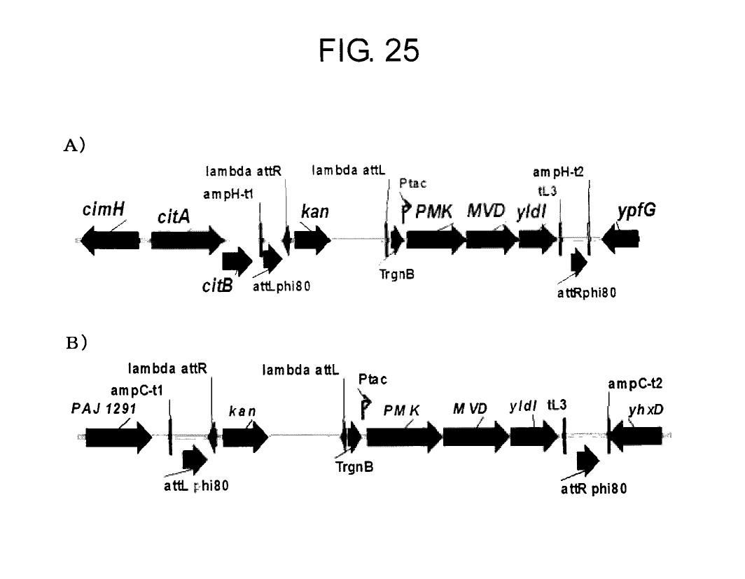

The plasmid pAH162-Km-Ptac-KDyI was integrated into a chromosome of the strain SC17(0) ΔampH::attB_{φ80} ΔampC::attB_{φ80} Δcrt::P_{tac}-mvk (M. paludicola)/pAH123-cat according to the previously reported protocol (Andreeva I G et al. FEMS Microbiol Lett. 2011; 318(1): 55-60, which is incorporated herein by reference in its entirety). After electrophoresis, cells were seeded on the LB agar containing 50 mg/L of kanamycin. Grown Km^{R} clones were tested by the polymerase chain reactions using synthesized oligonucleotides consisting of the nucleotide sequences of SEQ ID NO:163 (primer 13) and SEQ ID NO:167 (primer 17), and SEQ ID NO:163 (primer 13) and SEQ ID NO:169 (primer 19) as the primers. A strain holding the plasmid pAH162-Km-Ptac-KDyI integrated into ΔampC::attB_{φ80} or pC::attB_{φ80}m was selected. Maps of the modified chromosomals ΔampH:: pAH162-Km-Ptac-KDyI and ΔampC:: pAH162-Km-Ptac-KDyI are shown in FIG. 25 (A and B).

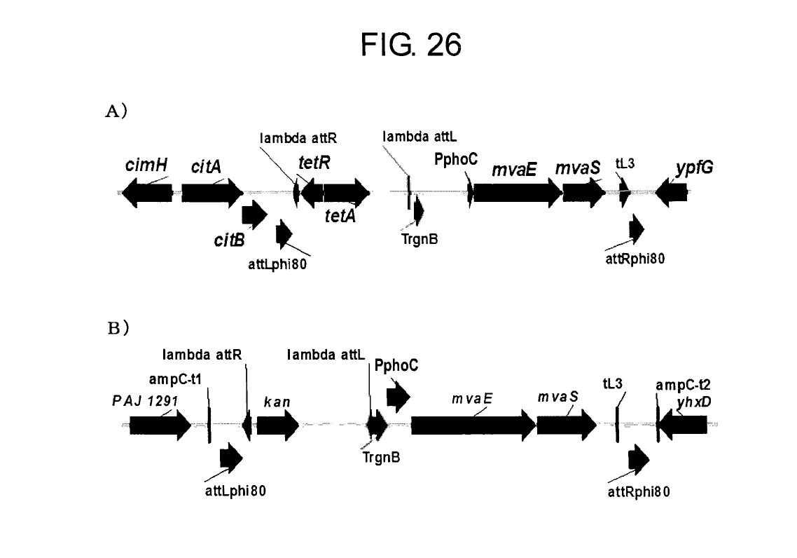

pAH162-PphoC-mvaES was inserted into a chromosome of recipient strains SC17(0) ΔampC::pAH162-Km-P_{tac}-KDyI ΔampH::attBₚₕᵢ₈₀ Δcrt::P_{tac}-mvk (M. paludicola) and SC17(0) ΔampC::attBₚₕᵢ₈₀ ΔampH::pAH162-Km-P_{tac}-KDyI Δcrt::P_{tac}-mvk (M. paludicola) using the helper plasmid pAH123-cat according to the previously reported protocol (Andreeva I G et al. FEMS Microbiol Lett. 2011; 318(1): 55-60, which is incorporated herein by reference in its entirety). As a result, two sets of strains designated asSWITCH-PphoC-1 and SWITCH-PphoC-2 were obtained. Maps of the modified chromosomes ΔampH::pAH162-PphoC-mvaES and ΔampC::pAH162-PphoC-mvaES are shown in FIG. 26.

The tetracycline resistant marker and the kanamycin resistant marker were removed from SWITCH-PphoC-1 by the phage phi80 Int/Xis dependent removal according to the previously reported technique (Katashkina J I et al. BMC Mol. Biol. 2009; 10: 34, which is incorporated herein by reference in its entirety). The resulting strain was designated as strain SWITCH-PphoC-1(S).

### Example 7

### Preparation of Pantoea Strain in which Ppa Gene of E. coli MG1655 (b4226) is Expressed at High Level in Strain SWITCH-PphoC-1(S) and Production of Isoprene by Cultivation Using the Same

A strain in which the expression of the ppa gene (pyrophosphate phosphatase gene) of E. coli MG1655 (b4226) was augmented by a strong promoter in P. ananatis strain was made by the following procedure.

(7-1) Construction of pAH162-Ptac-MG-Ppa

The ppa gene (b4226) was isolated by PCR using genomic DNA isolated from E. coli strain MG1655 as the template and using synthesized oligonucleotides consisting of the nucleotide sequences of SEQ ID NO:175 and SEQ ID NO:176 as the primers. The ppa gene isolated by PCR was introduced into a site cleaved with the restriction enzyme SmaI in the vector pSTV28-Ptac-Ttrp (Reference Example 3) using the in-fusion method to construct a plasmid pSTV28-Ptac-MG-ppa-Ttrp. Then, a region comprising the Tac promoter, the ppa gene and the Trp terminator was isolated using this plasmid as the template and using synthesized oligonucleotides consisting of the nucleotide sequences of SEQ ID NO:177 and SEQ ID NO:178 as the primers, and introduced into a site cleaved with the restriction enzymes BamHI and EcoRI in the vector pAH162-Km-attLR using In-Fusion HD Cloning Kit (supplied from Clontech) to construct a plasmid pAH162-Ptac-MG-ppa.

(7-2) Construction of Isoprene-Producing Strain in which Expression of Ppa Gene Derived from E. coli MG1655 is Enhanced and Construction of Control Strain

First, a derivative of P. ananatis SC17(0) possessing the attB site of phi80 that substituted a ydcI gene (PAJ_1320, Hara Y et al., The complete genome sequence of Pantoea ananatis AJ13355, an organism with great biotechnological potential. Appl. Microbiol. Biotechnol. 2012 January; 93(1):331-41, which is incorporated herein by reference in its entirety) was constructed. In order to obtain this strain, a PCR-amplified DNA fragment possessing attLphi80-kan-attRphi80 was obtained at a site flanking to a 40 bp region homologous to a ydcI target site in the genome using synthesized oligonucleotides consisting of the nucleotide sequences of SEQ ID NO:179 and SEQ ID NO:180 as the primers and using the genome of P. ananatis SC17(0) as the template. Subsequently, the λRed dependent integration of the DNA fragment was carried out according to previously reported technique (Katashkina J I et al., BMC Mol Biol. 2009; 10: 34, which is incorporated herein by reference in its entirety). After the electroporation, cells were cultured on the LB agar containing 50 mg/L of kanamycin. The substitution of the ydcI gene with attLphi80-kan-attRphi80 was confirmed using the primers consisting of the nucleotide sequences of SEQ ID NO:181 and SEQ ID NO:182. Then, a strain SC17(0)ΔydcI::Ptac-MG-ppa having the integrated plasmid pAH162-Ptac-MG-ppa using the helper plasmid pAH123 cat was made according to the previously reported technique (Andreeva I G et al., FEMS Microbiol Lett. 2011; 318(1): 55-60, which is incorporated herein by reference in its entirety). Genomic DNA isolated from this strain was electroporated into the strain SWITCH-PphoC-1(S) according to the previously reported method of chromosome electroporation (Katashkina J I et al., BMC Mol Biol. 2009; 10: 34, which is incorporated herein by reference in its entirety). That the modified genome ΔydcI::Ptac-MG-ppa had been integrated into the chromosome of the strain SWITCH-PphoC-1(S) was verified by PCR using the primers consisting of the nucleotide sequences of SEQ ID NO:182 and SEQ ID NO:183. The strain SWITCH-PphoC-1(S)ΔydcI:Ptac-MG-ppa that was the isoprene-producing strain in which the ppa gene derived from E. coli MG1655 had been introduced was obtained in this way.

Then, genomic DNA was isolated from the P. ananatis SC17(0) derivative possessing the attB site of phi80 that substituted the ydcI gene, and electroporated into the strain SWITCH-PphoC-1(S) according to the electroporation method of chromosomes previously reported (Katashkina J I et al., BMC Mol Biol. 2009; 10: 34, which is incorporated herein by reference in its entirety). The modified genome ΔydcI was verified by PCR using the primers consisting of the nucleotide sequences of SEQ ID NO:181 and SEQ ID NO:182, and then, an isoprene-producing strain SWITCH-PphosC-1(S) ΔydcI for control was chosen.

### (7-3) Confirmation of Expression Amount of Pyrophosphate Phosphatase

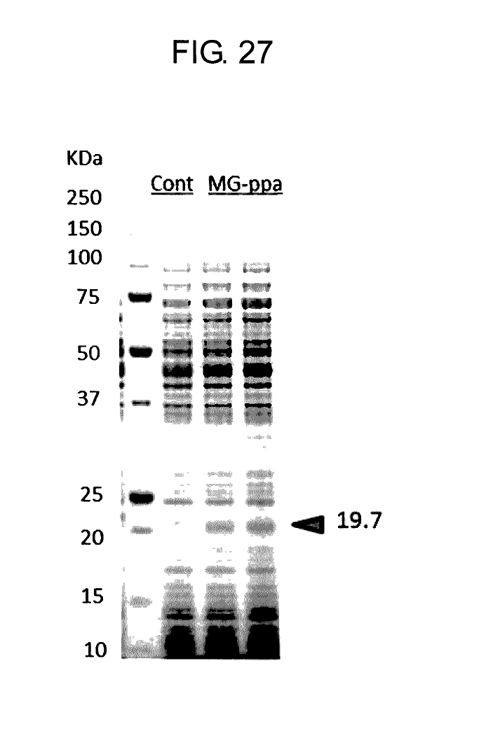

The expression amount of a protein of pyrophosphate phosphatase (PPA) in the strain SWITCH-PphoC-1(S)ΔydcI::Ptac-MG-ppa was confirmed on SDS-PAGE. Microbial cells from this strain were cultured with shaking in 3 mL of LB medium containing 50 mg/L of kanamycin at 30° C. overnight. The microbial cells were collected, subsequently washed twice with ice-cooled 50 mM Tris buffer (Tris-HCl, pH 8.0), and disrupted using the multibead shocker (Yasui Kikai, Japan, 4° C., On for 60 seconds and OFF for 60 seconds, 2500 rpm, 5 cycles). A solution of the disrupted cells was centrifuged at 14,000 rpm for 20 minutes to remove cell debris. The resulting supernatant fraction was handled as a soluble protein fraction. The soluble protein fraction was quantified by the BCA method, and 5 μg of the soluble protein was electrophoresed on SDS-PAGE (supplied from Invitrogen, NuPAGE: SDS-PAGE Gel System). Subsequently, CBB staining and decoloration were carried out according to the standard methods. A photograph of a gel showing around the mass of the PPA protein was shown in FIG. 27. As a result, the increase of the expression amount of the protein presumed to be PPA was confirmed in the strain SWITCH-PphoC-1(S)ΔydcI::Ptac-MG-ppa.

### (7-4) Introduction of Isoprene Synthase-Expressing Plasmid

Electrocells of the strains SWITCH-PphoC-1(S) ΔydcI::Ptac-MG-ppa and SWITCH-PphoC-1(S) ΔydcI (control) were prepared according to the standard method, pSTV28-Ptac-ispSM (US2014113344A1) was introduced thereto, and the cells were evenly applied onto the LB plate containing 60 mg/L of chloramphenicol and cultured 37° C. for 18 hours. Subsequently, transformants that exhibited resistance to chloramphenicol were obtained from the resulting plates. The resulting isoprene-producing bacterial strains were designated as SWITCH-PphoC-1(S)ydcI:MG-PPA/ispSM and SWITCH-PphoC-1(S)ΔydcI/ispSM (Control), respectively.

### (7-5) Measurement of Isoprene Synthase Activity

Microbial cells of the strains SWITCH-PphoC-1(S)ydcI::MG-PPA/ispSM and SWITCH-PphoC-1(S)ΔydcI/ispSM (Control) were evenly applied onto each LB plate containing chloramphenicol, and cultured at 34° C. for 16 to 24 hours. One loopful of the microbial cells from the resulting plate was inoculated to 1 mL of PS medium in a headspace vial (supplied from Perkin Elmer, 22 mL CLEAR CRIMP TOP VIAL cat#B0104236), then tightly sealed with a cap for the headspace vial with butyl rubber septum (supplied from Perkin Elmer, CRIMPS cat#B0104240), and cultured in a reciprocal shaking cultivation apparatus (120 rpm) at 30° C. for 48 hours.

Compositions of the PS medium are as described in Table 23.

TABLE 23
Composition of PS medium

| | | |
|---|---|---|
| Glucose | 4 | g/L |
| MgSO₄ 7H₂O | 1 | g/L |
| (NH₄)₂SO₄ | 10 | g/L |
| Yeast Extract | 50 | mg/L |
| FeSO₄ 7H₂O | 5 | mg/L |
| MnSO₄ 5H₂O | 5 | mg/L |
| KH₂PO₄ | 10 | mM |
| MES | 20 | mM pH 7.0 |

After termination of the cultivation, a concentration of isoprene in the headspace in the vial was measured by gas chromatography

TABLE 24
Amounts of isoprene formed after cultivation for 48 hours.

| | Amount of |
|---|---|
| Bacterial strain name | formed isoprene (mg/L) |
| SWITCH-PphoC-1(S)ΔydcI/ispSM (Control) | 80.2 ± 7.4 |
| SWITCH-PphoC-1(S)ydcI::MG-PPA/ispSM | 128.0 ± 5.9 |

From the result in Table 24, the strain SWITCH-PphoC-1(S)ydcI::MG-PPA/ispSM having the introduced ppa gene of E. coli MG1655 exhibited a higher activity of forming isoprene than the strain SWITCH-PphoC-1(S)ΔydcI/ispSM (Control).

### Example 8

### Production of Polyisoprene

Isoprene is collected with a trap cooled with liquid nitrogen by passing the fermentation exhaust. Collected isoprene is mixed with 35 g of hexane (Sigma-Aldrich) and 10 g of silica gel (Sigma-Aldrich, catalog No. 236772) and 10 g of alumina (Sigma-Aldrich, catalog No. 267740) under a nitrogen atmosphere in 100 mL glass vessel that is sufficiently dried. Resulting mixture is left at room temperature for 5 hours. Then supernatant liquid is skimmed and is added into 50 ml glass vessel that is sufficiently dried.

Meanwhile, in a glove box under a nitrogen atmosphere, 40.0 μmol of tris[bis(trimethylsilyl)amido]gadolinium, 150.0 μmol of tributylaluminium, 40.0 μmol of bis[2-(diphenylphosphino)phenyl]amine, 40.0 mol of triphenylcarbonium tetrakis(pentafluorophenyl)borate (Ph3CBC6F5)4) are provided in a glass container, which was dissolved into κ mL of toluene (Sigma-Aldrich, catalog No. 245511), to thereby obtain a catalyst solution. After that, the catalyst solution is taken out from the glove box and added to the monomer solution, which is then subjected to polymerization at 50° C. for 120 minutes.

After the polymerization, 1 mL of an isopropanol solution containing, by 5 mass %, 2,2′-methylene-bis(4-ethyl-6-t-butylphenol) (NS-5), is added to stop the reaction. Then, a large amount of methanol is further added to isolate the polymer, and the polymer is vacuum dried at 70° C. to obtain a polymer.

### Example 9

### Production of Rubber Compound

The rubber compositions formulated as shown in Table 25 are prepared, which are vulcanized at 145° C. for 35 minutes.

TABLE 25
Rubber compositions of Example 6

| | Parts by mass |
|---|---|
| Polyisoprene | 100 |
| Stearic Acid | 2 |
| Carbon Black (HAF class) | 50 |
| Antioxidant (*1) | 1 |
| Zinc Oxide | 3 |
| Cure Accelerator (*2) | 0.5 |
| Sulfur | 1.5 |

(*1) N-(1,3-dimethylbutyl)-N′-p-phenylenediamine
(*2) N-cyclohexyl-2-benzothiazolesulfenamide

### INDUSTRIAL APPLICABILITY

The isoprene synthase-expressing microorganism of the present invention is useful for production of isoprene.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An isoprene synthase-expressing microorganism, which exhibits improved expression of pyrophosphate phosphatase.

2. The isoprene synthase-expressing microorganism according to claim 1, wherein said microorganism is a microorganism transformed with an expression vector for isoprene synthase.

3. The isoprene synthase-expressing microorganism according to claim 1, wherein said pyrophosphate phosphatase is homologous to said microorganism.

4. The isoprene synthase-expressing microorganism according to claim 3, wherein expression of said pyrophosphate phosphatase is improved by modification of a promoter region of a pyrophosphate phosphatase gene inherent to said microorganism.

5. The isoprene synthase-expressing microorganism according to claim 1, wherein the expression of said pyrophosphate phosphatase is improved by an increased copy number of the pyrophosphate phosphatase gene on a chromosome.

6. The isoprene synthase-expressing microorganism according to claim 1, wherein said microorganism is a microorganism belonging to Enterobacteriaceae.

7. The isoprene synthase-expressing microorganism according to claim 1, wherein said microorganism has an ability to synthesize dimethylallyl diphosphate via a methylerythritol diphosphate pathway.

8. The isoprene synthase-expressing microorganism according to claim 7, wherein said microorganism is a bacterium belonging to genus Escherichia.

9. The isoprene synthase-expressing microorganism according to claim 8, wherein said bacterium belonging to genus Escherichia is Escherichia coli.

10. The isoprene synthase-expressing microorganism according to claim 1, wherein said microorganism has an ability to synthesize dimethylallyl diphosphate via a mevalonate pathway.

11. The isoprene synthase-expressing microorganism according to claim 1, wherein said microorganism is a bacterium belonging to genus Pantoea.

12. The isoprene synthase-expressing microorganism according to claim 11, wherein said bacterium belonging to genus Pantoea is Pantoea ananatis.

13. A method of producing an isoprene monomer, comprising culturing an isoprene synthase-expressing microorganism according to claim 1 in culture medium.

14. A method of producing an isoprene polymer, comprising:
(I) producing an isoprene monomer by the method according to claim 13; and
(II) polymerizing said isoprene monomer to produce said isoprene polymer.

15. A method for producing a rubber composition, comprising:
(A) preparing an isoprene polymer by the method of claim 14; and
(B) mixing said isoprene polymer with one or more rubber composition components.

16. A method for producing a tire, comprising:
(i) producing a rubber composition by the method of claim 15; and
(ii) forming said rubber composition into a tire.


SEQUENCE LISTING (184 sequences)

SEQ ID NO: 1 - DNA, 2414 bp, Artificial Sequence
  Genomic fragment containing lambda attL (Lambda phage attachment site-Left) - Tet (tetracycline-resistance marker) - lambda attR (Lambda phage attachment site-Right) -Ptac (tac promoter)
tgaagcctgcttttttatactaagttggcattataaaaaagcattgcttatcaatttgttgcaacgaacaggtcactatcagtcaaaataaaatcattatttgatttcgaattccccggatccgtcgacctgcagggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaatgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcgaatttattgcagatagtagtgcatttgcctttttagcgtttatatctgaaggttggttagatttccctgttttaattttattggctggtggtgggatcgctttacctgcattacagggagtgatgtctatccaaacaaagagtcatgagcaaggtgctttacagggattattggtgagccttaccaatgcaaccggtgttattggcccattactgtttactgttatttataatcattcactaccaatttgggatggctggatttggattattggtttagcgttttactgtattattatcctgctatcgatgaccttcatgttaacccctcaagctcaggggagtaaacaggagacaagtgcttagttatttcgtcaccaaatgatgttattccgcgaaatataatgaccctcttgataacccaagagggcattttttacgataaagaagatttagcttctgcagtctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaacttgagcgagatctccctgttgacaattaatcatcggctctataatgtgtggaatcgtgagcggataacaatttcacacaaggagactgcc

SEQ ID NO: 2 - DNA, 89 bp, Artificial Sequence
  Primer 1 for replacing a promoter region of ppa gene
ttgtaatcgctttcatctcactatgaaaaatgcggctacggttatggattttcctgctcttgaagcctgcttttttatactaagttggc

SEQ ID NO: 3 - DNA, 89 bp, Artificial Sequence
  Primer 2 for replacing a promoter region of ppa gene
aataacaacgtagatgtcttccggcagatctttacccgcagggacgttgagtaagctcatagctgtttcctgtgtgaaattgttatccg

SEQ ID NO: 4 - DNA, 24 bp, Artificial Sequence
  Primer 1 for confirming replacement of a promoter region of ppa gene with tac promoter
acagattgcgttacatcacacttc

SEQ ID NO: 5 - DNA, 24 bp, Artificial Sequence
  Primer 2 for confirming replacement of a promoter region of ppa gene with tac promoter
gtatttgatcggatctgcgttagc

SEQ ID NO: 6 - DNA, 1785 bp, Mucuna bracteata
  CDS (1)..(1785)
atggcaaccaacccttcatgcttatctactccatttttgtcctccacaccagcactaagtactagatttccattaagtgagaacttcacacaaaaaacatctcttgtcaatcccaaaccttggccacttatttctgcagtcagctctcaatttagccaaatagcagaagataatagtcgtcgttcagctaattaccacccaaacctctgggattttgaatttctgcagtctctcgaaaatgactctaagatggaaaagctggaagagaaagcaacaaagttggaggaggaagtgcgaaacatgatgaacgaagcaaagacagaagcactaagcttattggaattgatagacgacgtccagcgtctgggattgacctacaagtttgagaaggacataatcaaagcccttgagaagattgttccattggatgagagtgggctgcatgttacttctctcagcttccgtatacttagacaacatggctttgaggtttcccaagatgtgtttaagagatttaaggacaaggagggaggtttttgtgctgaacttaaagacgatgttcaagggttgctaagtctatatgaagcatcctatcttggttttgagggagaaagtctcttagacgaggcaagggcattttcaataacacatctcaagaacaacctaaacaaaggaataaacaccaaagtagcccaacaagttagccatgcactggaacttccttatcatcgaagactgcatagactggaagcacgatggctccttgacaaatatgaaccaaaggaaccccaccatcatttactacacgagcttgcaaagttggatttcaatttggtccaatcattgtaccagaaagagttgcgagaattgtcactgtggtggagggagattgggctcacaagcaagttggactttgttcgagacagattaatggaagtgtacttttgggcgctgggaatggcacctgatcctcaatttagtgaatgtcgtaaagtcgtcactaaaatgtttgggctagttactatcatcgatgatgtatatgacgtttacggtactttggacgagctacaactcttcaccgatgctgttgagagatgggacgtgaatgcgataaatacacttccagactatatgaaattgtgctatttagccctttataacaccgtcaatgacacagcttatagcatccttaaagaaaagggacataacaacatttcttatttgacaaaatcttggtgtgagttgtgcaaagcattcctccaagaagcaaaatggtcaaacaacaaaatcattccagcattcaacaagtacctagacaatgcatcggtgtcctcctctggtgtggctttgcttgctccttcctacttcttagtgtgccaagaacaagacatttcagaccaagctcttcattccttaactaatttccatggccttgtgcgttcatcatgcaccatttttaggctttgcaatgatctggctacctcatcggctgagctagagagaggtgaaacaacaaattcaatcacatcgtacatgcatgagaatgagacttctgaggagcaagcatgtaaggagttgagaaatttgatcgatgcagagtggaagaagatgaatgaagagcgagtttcaaattctacactcccaaaagcatttagggaaatagctattaacatggctcggatttcccattgcacataccaatatggagacggacttggaaggcccgactacaccacagagaacaggataaagttgctactaatagacccttttccaattaattag

SEQ ID NO: 7 - protein, 594 aa, Mucuna bracteata
MATNPSCLSTPFLSSTPALSTRFPLSENFTQKTSLVNPKPWPLISAVSSQFSQIAEDNSRRSANYHPNLWDFEFLQSLENDSKMEKLEEKATKLEEEVRNMMNEAKTEALSLLELIDDVQRLGLTYKFEKDIIKALEKIVPLDESGLHVTSLSFRILRQHGFEVSQDVFKRFKDKEGGFCAELKDDVQGLLSLYEASYLGFEGESLLDEARAFSITHLKNNLNKGINTKVAQQVSHALELPYHRRLHRLEARWLLDKYEPKEPHHHLLHELAKLDFNLVQSLYQKELRELSLWWREIGLTSKLDFVRDRLMEVYFWALGMAPDPQFSECRKVVTKMFGLVTIIDDVYDVYGTLDELQLFTDAVERWDVNAINTLPDYMKLCYLALYNTVNDTAYSILKEKGHNNISYLTKSWCELCKAFLQEAKWSNNKIIPAFNKYLDNASVSSSGVALLAPSYFLVCQEQDISDQALHSLTNFHGLVRSSCTIFRLCNDLATSSAELERGETTNSITSYMHENETSEEQACKELRNLIDAEWKKMNEERVSNSTLPKAFREIAINMARISHCTYQYGDGLGRPDYTTENRIKLLLIDPFPIN

SEQ ID NO: 8 - protein, 608 aa, Pueraria montana
MATNLLCLSNKLSSPTPTPSTRFPQSKNFITQKTSLANPKPWRVICATSSQFTQITEHNSRRSANYQPNLWNFEFLQSLENDLKVEKLEEKATKLEEEVRCMINRVDTQPLSLLELIDDVQRLGLTYKFEKDIIKALENIVLLDENKKNKSDLHATALSFRLLRQHGFEVSQDVFERFKDKEGGFSGELKGDVQGLLSLYEASYLGFEGENLLEEARTFSITHLKNNLKEGINTKVAEQVSHALELPYHQRLHRLEARWFLDKYEPKEPHHQLLLELAKLDFNMVQTLHQKELQDLSRWWTEMGLASKLDFVRDRLMEVYFWALGMAPDPQFGECRKAVTKMFGLVTIIDDVYDVYGTLDELQLFTDAVERWDVNAINTLPDYMKLCFLALYNTVNDTSYSILKEKGHNNLSYLTKSWRELCKAFLQEAKWSNNKIIPAFSKYLENASVSSSGVALLAPSYFSVCQQQEDISDHALRSLTDFHGLVRSSCVIFRLCNDLATSAAELERGETTNSIISYMHENDGTSEEQAREELRKLIDAEWKKMNRERVSDSTLLPKAFMEIAVNMARVSHCTYQYGDGLGRPDYATENRIKLLLIDPFPINQLMYV

SEQ ID NO: 9 - DNA, 1827 bp, Pueraria montana
atggcaaccaaccttttatgcttgtctaataaattatcgtcccccacaccaacaccaagtactagatttccacaaagtaagaacttcatcacacaaaaaacatctcttgccaatcccaaaccttggcgagttatttgtgctacgagctctcaatttacccaaataacagaacataatagtcggcgttcagctaattaccagccaaacctctggaattttgaatttctgcagtctctggaaaatgaccttaaggtggaaaaactagaagagaaggcaacaaagctagaggaggaggtacgatgcatgatcaacagagtagacacacaaccattaagcttactagaattgatcgacgatgtccagcgtctaggattgacctacaagtttgagaaggacataatcaaagcccttgagaatattgttttgctggatgagaataagaaaaataaaagtgacctccatgctactgctctcagcttccgtttacttagacaacatggctttgaggtttcccaagatgtgtttgagagatttaaggacaaggagggaggtttcagtggtgaacttaaaggtgatgtgcaagggttgctgagtctatatgaagcatcctatcttggctttgagggagaaaatctcttggaggaggcaaggacattttcaataacacatctcaagaacaacctaaaagaaggaataaacaccaaagtggcagaacaagttagtcatgcactggaacttccctatcatcaaagattgcatagactagaagcacgatggttccttgacaaatatgaaccaaaggaaccccaccatcagttactactcgagcttgcaaagctagatttcaatatggtgcaaacattgcaccagaaagaactgcaagacctgtcaaggtggtggacggagatggggctagcaagcaagctagactttgtccgagacagattaatggaagtgtatttttgggcgttgggaatggcacctgatcctcaattcggtgaatgtcgtaaagctgtcactaaaatgtttggattggtcaccatcatcgatgatgtatatgacgtttatggtactttggatgagctacaactcttcactgatgctgttgagagatgggacgtgaatgccataaacacacttccagactacatgaagttgtgcttcctagcactttataacaccgtcaatgacacgtcttatagcatccttaaagaaaaaggacacaacaacctttcctatttgacaaaatcttggcgtgagttatgcaaagcattccttcaagaagcaaaatggtcgaacaacaaaatcattccagcatttagcaagtacctggaaaatgcatcggtgtcctcctccggtgtggctttgcttgctccttcctacttctcagtgtgccaacaacaagaagatatctcagaccatgctcttcgttctttaactgatttccatggccttgtgcgctcctcatgcgtcattttcagactctgcaatgatttggctacctcagcggctgagctagagaggggtgagacgacaaattcaataatatcttatatgcatgagaatgacggcacttctgaagagcaagcacgtgaggagttgagaaaattgatcgatgcagagtggaagaagatgaaccgagagcgagtttcagattctacactactcccaaaagcttttatggaaatagctgttaacatggctcgagtttcgcattgcacataccaatatggagacggacttggaaggccagactacgccacagagaatagaatcaagttgctacttatagacccctttccaatcaatcaactaatgtacgtgtaa

SEQ ID NO: 10 - DNA, 1695 bp, Artificial Sequence
  Polynucleotide having modified codons, which encodes isoprene synthase derived from Pueraria montana (IspSK gene)
atgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaa

SEQ ID NO: 11 - protein, 595 aa, Populus alba x Populus tremula
MATELLCLHRPISLTHKLFRNPLPKVIQATPLTLKLRCSVSTENVSFTETETEARRSANYEPNSWDYDFLLSSDTDESIEVYKDKAKKLEAEVRREINNEKAEFLTLLELIDNVQRLGLGYRFESDIRRALDRFVSSGGFDGVTKTSLHATALSFRLLRQHGFEVSQEAFSGFKDQNGNFLENLKEDTKAILSLYEASFLALEGENILDEARVFAISHLKELSEEKIGKELAEQVNHALELPLHRRTQRLEAVWSIEAYRKKEDANQVLLELAILDYNMIQSVYQRDLRETSRWWRRVGLATKLHFAKDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLIFAYFAVVQNIKKEEIENLQKYHDIISRPSHIFRLCNDLASASAEIARGETANSVSCYMRTKGISEELATESVMNLIDETCKKMNKEKLGGSLFAKPFVETAINLARQSHCTYHNGDAHTSPDELTRKRVLSVITEPILPFER

SEQ ID NO: 12 - DNA, 1788 bp, Populus alba x Populus tremula
atggcaactgaattattgtgcttgcaccgtccaatctcactgacacacaaactgttcagaaatcccttacctaaagtcatccaggccactcccttaactttgaaactcagatgttctgtaagcacagaaaacgtcagcttcacagaaacagaaacagaagccagacggtctgccaattatgaaccaaatagctgggattatgattttttgctgtcttcagacactgacgaatcgattgaagtatacaaagacaaggccaaaaagctggaggctgaggtgagaagagagattaacaatgaaaaggcagagtttttgactctgcttgaactgatagataatgtccaaaggttaggattgggttaccggttcgagagtgacataaggagagccctcgacagatttgtttcttcaggaggatttgatggtgttacaaaaactagccttcatgctactgctcttagcttcaggcttctcagacagcatggctttgaggtctctcaagaagcgttcagtggattcaaggatcaaaatggcaatttcttggaaaaccttaaggaggacaccaaggcaatactaagcctatatgaagcttcatttcttgcattagaaggagaaaatatcttggatgaggccagggtgtttgcaatatcacatctaaaagagctcagcgaagaaaagattggaaaagagctggccgaacaggtgaatcatgcattggagcttccattgcatcgcaggacgcaaagactagaagctgtttggagtattgaagcataccgtaaaaaggaagatgcaaatcaagtactgctagaacttgctatattggactacaacatgattcaatcagtataccaaagagatcttcgcgagacatcaaggtggtggaggcgagtgggtcttgcaacaaagttgcattttgctaaagacaggttaattgaaagcttttactgggcagttggagttgcgttcgaacctcaatacagtgattgccgtaattcagtagcaaaaatgttttcatttgtaacaatcattgatgatatctatgatgtttatggtactctggatgagctggagctatttacagatgctgttgagagatgggatgttaacgccatcaatgatcttccggattatatgaagctctgcttcctagctctctacaacactatcaatgagatagcttatgacaatctgaaggacaagggggaaaacattcttccatacctaacaaaagcgtgggcagatttatgcaatgcattcctacaagaagcaaaatggctgtacaataagtccacaccaacatttgatgactatttcggaaatgcatggaaatcatcctcagggcctcttcaactaatttttgcctactttgccgtggttcaaaacatcaagaaagaggaaattgaaaacttacaaaagtatcatgatatcatcagtaggccttcccacatctttcgtctttgcaacgacctggcttcagcatcggctgagatagcgagaggtgaaactgcgaattccgtatcctgctacatgcgtacaaaaggcatttctgaggaacttgctactgaatccgtaatgaatttgatcgacgaaacctgtaaaaagatgaacaaagaaaagcttggtggctctttgtttgcaaaaccttttgtcgaaacagctattaaccttgcacggcaatcccattgcacttatcataacggagatgcgcatacttcaccagacgagctaactaggaaacgtgtcctgtcagtaatcacagagcctattctaccctttgagagataa

SEQ ID NO: 13 - DNA, 1680 bp, Artificial Sequence
  Polynucleotide having modified codons, which encodes isoprene synthase derived from Populus alba x Populus tremula (IspSP gene)
atgtgctctgtttctaccgagaacgtttccttcactgagacggaaaccgaggcacgtcgtagcgcgaactacgagccgaatagctgggactacgatttcctgctgtcttccgatactgacgaatctattgaggtgtacaaagacaaagcaaagaaactggaggctgaagtgcgccgcgaaattaacaacgagaaagctgaattcctgactctgctggagctgatcgataacgtacagcgcctgggtctgggttaccgcttcgaatctgatatccgtcgcgcactggatcgtttcgtaagcagcggcggtttcgatggcgtgaccaaaacgagcctgcacgctaccgcgctgtccttccgtctgctgcgtcagcacggcttcgaagtttctcaggaagcattctccggtttcaaagatcaaaacggtaacttcctggaaaacctgaaagaagacactaaggcgatcctgagcctgtatgaggcaagctttctggccctggagggtgagaacatcctggatgaggcgcgcgtattcgccatctcccatctgaaagagctgtctgaagagaaaatcggtaaggaactggcagagcaggttaatcacgcactggaactgccgctgcatcgtcgtacccagcgtctggaggcggtttggtccatcgaagcgtaccgcaaaaaggaggatgctaaccaggttctgctggaactggccatcctggactacaacatgatccagtccgtttaccagcgtgatctgcgtgaaacctcccgttggtggcgccgtgtgggcctggcgaccaaactgcacttcgctaaggaccgcctgattgagtctttttactgggcagtcggcgttgcgttcgaacctcagtattctgactgccgtaacagcgttgcgaaaatgttcagcttcgttactattatcgacgacatctacgacgtttacggtactctggacgagctggaactgtttaccgacgctgtcgaacgttgggatgttaacgccatcaacgatctgcctgactacatgaaactgtgcttcctggcactgtataacacgatcaacgaaattgcatacgacaacctgaaagacaaaggtgaaaacatcctgccgtacctgactaaagcgtgggcggatctgtgtaacgcttttctgcaagaagcgaaatggctgtataacaaatccactccgacctttgacgattatttcggcaatgcctggaaatccagctctggcccgctgcaactgatcttcgcttattttgcggttgtccaaaacatcaaaaaggaggaaattgaaaacctgcaaaaataccacgatatcattagccgtccttctcatatctttcgcctgtgcaacgacctggcaagcgcgtccgcagagatcgcacgtggcgaaaccgctaactctgtttcctgctacatgcgcaccaagggcatttccgaagagctggcaaccgagagcgtaatgaatctgatcgacgaaacctgtaagaaaatgaacaaagaaaaactgggtggctccctgttcgctaaaccgttcgtagagactgctattaacctggcacgtcagagccactgcacctaccacaatggtgacgcacatactagcccggatgaactgactcgtaaacgtgtactgtctgttatcaccgaaccgattctgccgttcgaacgttaa

SEQ ID NO: 14 - DNA, 1785 bp, Artificial Sequence
  Polynucleotide which encodes isoprene synthase derived from Mucuna bracteata and which is fused with chloroplast-localization signal (IspSM(L) gene)
atggctaccaacccgtcctgtctgtcaaccccgttcctgtcttcaaccccggctctgtccacccgcttcccgctgtccgaaaacttcacccagaaaacgagcctggttaacccgaaaccgtggccgctgatttctgcggtcagctctcagtttagtcaaatcgcggaagataattctcgtcgcagtgccaactatcatccgaatctgtgggattttgaattcctgcagtcgctggaaaacgacagcaaaatggaaaaactggaagaaaaagcgaccaaactggaagaagaagtgcgtaacatgatgaatgaagcgaaaacggaagccctgtctctgctggaactgattgatgacgttcaacgcctgggtctgacctacaaattcgaaaaagatatcatcaaagccctggaaaaaattgtcccgctggacgaatcaggtctgcacgtgacctccctgtcatttcgtatcctgcgccagcatggcttcgaagtttcgcaagatgtctttaaacgtttcaaagacaaagaaggcggtttctgcgcagaactgaaagatgacgtgcagggtctgctgtctctgtatgaagctagttacctgggttttgaaggcgaatccctgctggatgaagcgcgcgccttctcaattacccacctgaaaaacaatctgaacaaaggcatcaatacgaaagtggcacagcaagttagtcatgctctggaactgccgtatcaccgtcgcctgcatcgtctggaagcccgctggctgctggataaatacgaaccgaaagaaccgcatcaccatctgctgcacgaactggcaaaactggactttaatctggttcagtcgctgtatcaaaaagaactgcgtgaactgagcctgtggtggcgcgaaattggtctgacctctaaactggattttgtgcgtgaccgcctgatggaagtttacttctgggcactgggcatggctccggatccgcagtttagcgaatgccgtaaagtggttaccaaaatgttcggtctggtgacgattatcgatgacgtctatgatgtgtacggcaccctggacgaactgcaactgttcacggatgcagtcgaacgctgggacgtgaacgctatcaataccctgccggattatatgaaactgtgttatctggcactgtacaacaccgttaatgacacggcttacagcatcctgaaagaaaaaggtcataacaacatctcctacctgaccaaatcatggtgcgaactgtgtaaagcgtttctgcaggaagccaaatggtctaacaataaaattatcccggcgttcaacaaatatctggataatgccagtgttagttcctcaggcgtcgcactgctggctccgtcctactttctggtctgtcaggaacaagatatttcggaccaggcactgcacagcctgaccaactttcatggtctggttcgttcgagctgcaccatcttccgcctgtgtaatgatctggcgacgtctagtgccgaactggaacgtggcgaaaccacgaactccattacctcatatatgcacgaaaatgaaacgagtgaagaacaggcgtgcaaagaactgcgtaacctgatcgatgccgaatggaagaaaatgaacgaagaacgcgtgtcgaatagcaccctgccgaaagcctttcgtgaaattgcaatcaatatggctcgcatttcccattgtacgtatcagtacggcgatggtctgggccgcccggactacacgaccgaaaaccgcattaaactgctgctgattgacccgttcccgattaactga

SEQ ID NO: 15 - DNA, 1656 bp, Artificial Sequence
  Polynucleotide without chloroplast-localization signal, which encodes isoprene synthase derived from Mucuna bracteata (IspSM gene)
atgtccgccgtttcaagccagttctctcaaatcgccgaagacaatagccgtcgctcagcaaattatcatccgaatctgtgggactttgaatttctgcagtctctggaaaacgatagtaaaatggaaaaactggaagaaaaagccaccaaactggaagaagaagtgcgtaacatgatgaatgaagcgaaaacggaagccctgagcctgctggaactgattgatgacgtccaacgcctgggtctgacctacaaattcgaaaaagatatcatcaaagcactggaaaaaattgtcccgctggacgaatcaggtctgcacgtgacgtctctgagttttcgtatcctgcgccagcatggcttcgaagtttcgcaagatgtctttaaacgtttcaaagacaaagaaggcggtttctgcgcggaactgaaagatgacgtgcagggtctgctgtccctgtatgaagcctcatacctgggttttgaaggcgaatccctgctggatgaagcgcgcgccttctcaattacccacctgaaaaacaatctgaacaaaggcatcaatacgaaagtggcacagcaagttagccatgctctggaactgccgtatcaccgtcgcctgcatcgtctggaagcacgctggctgctggataaatacgaaccgaaagaaccgcatcaccatctgctgcacgaactggcgaaactggactttaatctggttcagtcgctgtatcaaaaagaactgcgtgaactgagcctgtggtggcgcgaaattggtctgacctctaaactggattttgtgcgtgaccgcctgatggaagtttacttctgggcactgggcatggctccggatccgcagtttagcgaatgccgtaaagtggttaccaaaatgttcggtctggtcacgattatcgatgacgtctatgatgtgtacggcaccctggacgaactgcaactgttcacggatgcggtcgaacgctgggacgtgaacgccatcaataccctgccggattatatgaaactgtgttatctggcgctgtacaacaccgttaatgacacggcctatagcatcctgaaagaaaaaggtcataacaacatctcgtacctgaccaaaagctggtgcgaactgtgtaaagcgtttctgcaggaagccaaatggtctaacaacaaaatcatcccggcattcaacaaatacctggataatgctagtgttagctctagtggcgtcgcactgctggctccgtcctactttctggtgtgtcaggaacaagatatttctgaccaggcgctgcacagtctgaccaactttcatggtctggttcgttcctcatgcaccatcttccgcctgtgtaatgatctggcgacgtcgagcgccgaactggaacgtggcgaaaccacgaactcgattaccagctatatgcacgaaaatgaaacgagtgaagaacaggcatgcaaagaactgcgtaacctgatcgatgctgaatggaagaaaatgaacgaagaacgcgtgtccaattcaaccctgccgaaagcctttcgtgaaattgcaatcaatatggctcgcatttcccattgtacgtatcagtacggcgatggtctgggccgcccggactacacgaccgaaaaccgtattaaactgctgctgattgacccgttcccgattaactaa

SEQ ID NO: 16 - DNA, 399 bp, Artificial Sequence
  Ptac-Ttrp
ggtaccagatctccctgttgacaattaatcatcggctctataatgtgtggaatcgtgagcggataacaatttcacacaaggagactcccgggagccgccagttccgctggcggcattttaactttctttaatgaagccggaaaaatcctaaattcatttaatatttatctttttaccgtttcgcttaccccggtcgaacgtcaacttacgtcatttttccgcccaacagtaatataatcaaacaaattaatcccgcaacataacaccagtaaaatcaataattttctctaagtcacttattcctcaggtaattgttaatatatccagaatgttcctcaaaatatattttccctctatcttctcgttgcgcttaatttgactaattctcattagggatcc

SEQ ID NO: 17 - DNA, 3383 bp, Artificial Sequence
  Expression plasmid pSTV28-Ptac-Ttrp
cgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtaccagatctccctgttgacaattaatcatcggctctataatgtgtggaatcgtgagcggataacaatttcacacaaggagactcccgggagccgccagttccgctggcggcattttaactttctttaatgaagccggaaaaatcctaaattcatttaatatttatctttttaccgtttcgcttaccccggtcgaacgtcaacttacgtcatttttccgcccaacagtaatataatcaaacaaattaatcccgcaacataacaccagtaaaatcaataattttctctaagtcacttattcctcaggtaattgttaatatatccagaatgttcctcaaaatatattttccctctatcttctcgttgcgcttaatttgactaattctcattagggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccaggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattt

SEQ ID NO: 18 - DNA, 79 bp, Artificial Sequence
  Primer for amplifying IspSK gene (Ptac-IspS(K)F)
gataacaatttcacacaataattttgtttaactttaagaaggagatataatgtgtgcgacctcttctcaatttactcag

SEQ ID NO: 19 - DNA, 42 bp, Artificial Sequence
  Primer for amplifying IspSK gene (IspS(K)R-MCSR)
acggccagtgaattcttagacatacatcagctggttaatcgg

SEQ ID NO: 20 - DNA, 79 bp, Artificial Sequence
  Primer for amplifying IspSP gene (Ptac-IspS(P)F)
gataacaatttcacacaataattttgtttaactttaagaaggagatataatgtgctctgtttctaccgagaacgtttcc

SEQ ID NO: 21 - DNA, 43 bp, Artificial Sequence
  Primer for amplifying IspSP gene (IspS(P)R-MCSR)
acggccagtgaattcttaacgttcgaacggcagaatcggttcg

SEQ ID NO: 22 - DNA, 69 bp, Artificial Sequence
  Primer for amplifying IspSM gene (Ptac-IspS(K)F)
gataacaatttcacacaataattttgtttaactttaagaaggagatataatgtccgccgtttcaagcca

SEQ ID NO: 23 - DNA, 35 bp, Artificial Sequence
  Primer for amplifying IspSM gene
acggccagtgaattcttagttaatcgggaacgggt

SEQ ID NO: 24 - DNA, 79 bp, Artificial Sequence
  Primer for amplifying IspSM(L) gene
gataacaatttcacacaataattttgtttaactttaagaaggagatataatggctaccaacccgtcctgtctgtcaacc

SEQ ID NO: 25 - DNA, 35 bp, Artificial Sequence
  Primer for amplifying IspSM(L) gene
acggccagtgaattctcagttaatcgggaacgggt

SEQ ID NO: 26 - DNA, 29 bp, Artificial Sequence
  Primer for amplifying IpSTV28-Ptac-Ttrp construct (pSTV28-F)
gtgtgaaattgttatccgctcacaattcc

SEQ ID NO: 27 - DNA, 27 bp, Artificial Sequence
  Primer for amplifying pSTV28-Ptac-Ttrp construct (pSTV28-R)
gaattcactggccgtcgttttacaacg

SEQ ID NO: 28 - DNA, 41 bp, Artificial Sequence
  Primer for amplifying dxs gene (dxs-F)
caggaaacagctatgagttttgatattgccaaatacccgac

SEQ ID NO: 29 - DNA, 28 bp, Artificial Sequence
  Primer for amplifying dxs gene (dxs-R)
gctgccactcctgctatactcgtcatac

SEQ ID NO: 30 - DNA, 30 bp, Artificial Sequence
  Primer for amplifying dxs gene (pMW219-F)
catagctgtttcctgtgtgaaattgttatc

SEQ ID NO: 31 - DNA, 40 bp, Artificial Sequence
  Primer for amplifying dxs gene (pMW219-R)
agcaggagtggcagcgaattcgagctcggtacccggggat

SEQ ID NO: 32 - DNA, 40 bp, Artificial Sequence
  Primer for amplifying ERG12 gene (MVK-IFS_5742-33-1)
acacaaggagactcccatgtcattaccgttcttaacttct

SEQ ID NO: 33 - DNA, 46 bp, Artificial Sequence
  Primer for amplifying ERG12 gene (MVK-IFA_5742-33-2)
ggaactggcggctcccgggttattatgaagtccatggtaaattcgt

SEQ ID NO: 34 - DNA, 38 bp, Artificial Sequence
  Primer for amplifying ERG8 gene (PMK-IFS_5742-33-3)
acacaaggagactcccatgtcagagttgagagccttca

SEQ ID NO: 35 - DNA, 46 bp, Artificial Sequence
  Primer for amplifying ERG8 gene (PMK-IFA_5742-33-4)
ggaactggcggctcccgggttattatttatcaagataagtttccgg

SEQ ID NO: 36 - DNA, 37 bp, Artificial Sequence
  Primer for amplifying ERG19 gene (MVD-IFS_5742-33-5)
acacaaggagactcccatgaccgtttacacagcatcc

SEQ ID NO: 37 - DNA, 46 bp, Artificial Sequence
  Primer for amplifying ERG19 gene (MVD-IFA_5742-33-6)
ggaactggcggctcccgggttattattcctttggtagaccagtctt

SEQ ID NO: 38 - DNA, 36 bp, Artificial Sequence
  Primer for amplifying IDI1 gene (yIDI-IFS_5742-33-7)
acacaaggagactcccatgccccatggtgcagtatc

SEQ ID NO: 39 - DNA, 49 bp, Artificial Sequence
  Primer for amplifying IDI1 gene (yIDI-IFA_5742-33-8)
ggaactggcggctcccgggttattatagcattctatgaatttgcctgtc

SEQ ID NO: 40 - DNA, 5892 bp, Artificial Sequence
  Nucleotide sequence of prepared plasmid pUC-mvk-pmk
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataaggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 41 - DNA, 39 bp, Artificial Sequence
  Primer for amplifying ERG12 gene (KKS1-6038-2-1)
tcgagctcggtacccatgtcattaccgttcttaacttct

SEQ ID NO: 42 - DNA, 53 bp, Artificial Sequence
  Primer for amplifying ERG12 gene (KKA1-6038-2-2)
ttaagggtgcaggcctatcgcaaattagcttatgaagtccatggtaaattcgt

SEQ ID NO: 43 - DNA, 50 bp, Artificial Sequence
  Primer for amplifying ERG8 gene (KKS2-6083-2-3)
ggcctgcacccttaaggaggaaaaaaacatgtcagagttgagagccttca

SEQ ID NO: 44 - DNA, 39 bp, Artificial Sequence
  Primer for amplifying ERG8 gene (KKA2-6083-2-4)
ctctagaggatccccttatttatcaagataagtttccgg

SEQ ID NO: 45 - DNA, 6135 bp, Artificial Sequence
  Nucleotide sequence of prepared plasmid pTWV-dmd-yidi
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaagaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagtaatgtgagttagctcactcattaggcaccccaggctttacactttatgcttccggctcgtatgttgtgtggaattgtgagcggataacaatttcacacaggaaacagctatgaccatgattacgaattcgagctcggtacccatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataaggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 46 - DNA, 36 bp, Artificial Sequence
  Primer for amplifying ERG19 gene (DyIS1-6083-2-5)
tcgagctcggtacccatgaccgtttacacagcatcc

SEQ ID NO: 47 - DNA, 60 bp, Artificial Sequence
  Primer for amplifying ERG19 gene (DyIA1-6083-2-6)
tttttttacctcctaagggcgatgcagcgaattgatcttattcctttggtagaccagtct

SEQ ID NO: 48 - DNA, 57 bp, Artificial Sequence
  Primer for amplifying IDI1 gene (DyIS2-6083-2-7)
taggaggtaaaaaaaaatgactgccgacaacaatagtatgccccatggtgcagtatc

SEQ ID NO: 49 - DNA, 42 bp, Artificial Sequence
  Primer for amplifying IDI1 gene (DyIA2-6083-2-8)
ctctagaggatccccttatagcattctatgaatttgcctgtc

SEQ ID NO: 50 - DNA, 9257 bp, Artificial Sequence
  Nucleotide sequence of prepared plasmid pTrc-KKDyI(beta))
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataactgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 51 - DNA, 36 bp, Artificial Sequence
  Primer (KKDS2_6038-3-2)
gaggaataaaccatgtcattaccgttcttaacttct

SEQ ID NO: 52 - DNA, 55 bp, Artificial Sequence
  Primer (KKMyIA_6038-2-9)
aagggcgaattctgcatgcagctaccttaagttatttatcaagataagtttccgg

SEQ ID NO: 53 - DNA, 51 bp, Artificial Sequence
  Primer (KMS_6038-6-1)
gcagaattcgcccttaaggaggaaaaaaaaatgaccgtttacacagcatcc

SEQ ID NO: 54 - DNA, 47 bp, Artificial Sequence
  Primer (KDyIA_6038-3-3)
ccatatggtaccagctgcagttatagcattctatgaatttgcctgtc

SEQ ID NO: 55 - DNA, 11386 bp, Artificial Sequence
  Nucleotide sequence of prepared plasmid pMW219-KKDyI-TaspA
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctgtttttccactcttcgttcactttcgccaggtagctggtgaagacgaaggaagtcccggagccatctgcgcggcgtactacagcaatgttttgtgaaggcagtttcagacccggattcagtttggcgatggcttcatcatcccacttcttgattttgcccaggtagatgtcgccgagggttttaccatccagcaccagttcgccagacttcagccctggaatgttaaccgccagcaccacgccgccaatcacggtcgggaactggaacagaccttcctgagccagtttttcgtcagacagcggcgcgtcagaggcaccaaaatcaacggtattagcgataatctgttttacgccaccggaagaaccgataccctggtagttaactttattaccggtttctttctggtaagtgtcagcccatttggcatacaccggcgcagggaaggttgcacctgcacctgtcaggcttgcttctgcaaacacagagaaagcactcatcgataaggtcgcggcgacaacagttgcgacggtggtacgcataactttcataatgtctcctgggaggattcataaagcattgtttgttggctacgagaagcaaaataggacaaacaggtgacagttatatgtaaggaatatgacagttttatgacagagagataaagtcttcagtctgatttaaataagcgttgatattcagtcaattacaaacattaataacgaagagatgacagaaaaattttcattctgtgacagagaaaaagtagccgaagatgacggtttgtcacatggagttggcaggatgtttgattaaaatgaagcctgcttttttatactaagttggcattataaaaaagcattgcttatcaatttgttgcaacgaacaggtcactatcagtcaaaataaaatcattatttgatttcgaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctccggataagtagacagcctgataagtcgcacgaaaaacaggtattgacaacatgaagtaacatgcagtaagatacaaatcgctaggtaacactagcagcgtcaaccgggcgctctagctagagccaagctagcttggccggatccgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgtcgaagcttaacacagaaaaaagcccgcacctgacagtgcgggctttttttttcgaccactgcagtctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaacttgagcggcccttgacgatgccacatcctgagcaaataattcaaccactaattgtgagcggataacacaaggaggaaacagctatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttccttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtctacaaataaaaaaggcacgtcagatgacgtgccttttttcttggggccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcatagctcacgctgtaggtatctcagttcggtgtaggtcgttcgctccaagctgggctgtgtgccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgccaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcactaccgcattaaagcatatcgatgataagctgtcaaacatgagcgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 56 - DNA, 12232 bp, Artificial Sequence
  Nucleotide sequence of the prepared plasmid pMW-Tn7-Pgi-KKDyI-TaspA-Tn7
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccctgtttttccactcttcgttcactttcgccaggtagctggtgaagacgaaggaagtcccggagccatctgcgcggcgtactacagcaatgttttgtgaaggcagtttcagacccggattcagtttggcgatggcttcatcatcccacttcttgattttgcccaggtagatgtcgccgagggttttaccatccagcaccagttcgccagacttcagccctggaatgttaaccgccagcaccacgccgccaatcacggtcgggaactggaacagaccttcctgagccagtttttcgtcagacagcggcgcgtcagaggcaccaaaatcaacggtattagcgataatctgttttacgccaccggaagaaccgataccctggtagttaactttattaccggtttctttctggtaagtgtcagcccatttggcatacaccggcgcagggaaggttgcacctgcacctgtcaggcttgcttctgcaaacacagagaaagcactcatcgataaggtcgcggcgacaacagttgcgacggtggtacgcataactttcataatgtctcctgggaggattcataaagcattgtttgttggctacgagaagcaaaataggacaaacaggtgacagttatatgtaaggaatatgacagttttatgacagagagataaagtcttcagtctgatttaaataagcgttgatattcagtcaattacaaacattaataacgaagagatgacagaaaaattttcattctgtgacagagaaaaagtagccgaagatgacggtttgtcacatggagttggcaggatgtttgattaaaatgaagcctgcttttttatactaagttggcattataaaaaagcattgcttatcaatttgttgcaacgaacaggtcactatcagtcaaaataaaatcattatttgatttcgaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctccggataagtagacagcctgataagtcgcacgaaaaacaggtattgacaacatgaagtaacatgcagtaagatacaaatcgctaggtaacactagcagcgtcaaccgggcgctctagctagagccaagctagcttggccggatccgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgtcgaagcttaacacagaaaaaagcccgcacctgacagtgcgggctttttttttcgaccactgcagtctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaacttgagcggcccttgacgatgccacatcctgagcaaataattcaaccactaattgtgagcggataacacaaggaggaaacagctatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttccttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtctacaaataaaaaaggcacgtcagatgacgtgccttttttcttggggccggggatcctctagagtcgaaagaaaaatgccccgcttacgcagggcatccatttattactcaaccgtaaccgattttgccaggttacgcggctggtcaacgtcggtgcctttgatcagcgcgacatggtaagccagcagctgcagcggaacggtgtagaagatcggtgcaatcacctcttccacatgcggcatctcgatgatgtgcatgttatcgctacttacaaaacccgcatcctgatcggcgaagacatacaactgaccgccacgcgcgcgaacttcttcaatgttggatttcagtttttccagcaattcgttgttcggtgcaacaacaataaccggcatatcggcatcaattagcgccagcggaccgtgtttcagttcgccagcagcgtaggcttcagcgtgaatgtaagagatctctttcaacttcaatgcgccttccagcgcgattgggtactgatcgccacggcccaggaacagcgcgtgatgtttgtcagagaaatcttctgccagcgcttcaatgcgtttgtcctgagacagcatctgctcaatacggctcggcagcgcctgcagaccatgcacgatgtcatgttcaatggaggcatccagacctttcaggcgagacagcttcgccaccagcatcaacagcacagttaactgagtggtgaatgctttagtggatgccacgccgatttctgtacccgcgttggtcattagcgccagatcggattcgcgcaccagagaagaacccggaacgttacagattgccagtgaaccaaggtaacccagctctttcgacagacgcaggccagccagggtatccgcggtttcgccagactgtgacaggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcatagctcacgctgtaggtatctcagttcggtgtaggtcgttcgctccaagctgggctgtgtgccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgccaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcactaccgcattaaagcatatcgatgataagctgtcaaacatgagcgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 57 - DNA, 9175 bp, Artificial Sequence
  Nucleotide sequence containing genomic sequence encoding downstream enzymes in the mevalonate pathway
ttagtacagcggcttaccgctactgtctttaatattggtcttccacgcagcgcgaacctgttcaactacactatccggcaggctggcgtaatccaggtcgttcgcctgtttagccccggttttgtacgcccagtcgaagaatttcagcacttctgtgccttgttctggtttcttctgatctttgtggatcagaatgaacgtggtagaggtaataggccatgcatcttcgcctttctggttggtcagatcctgagcgaaggttttgctccagtctgcaccttttgctgcattagcgaagttttcttcggtcggactaaccggtttaccatcagcggagatcagtttggtgtacgccaggttgttctgcttcgcgtaagcatattcaacataaccaattgcacccggcagacgctgaacgaacgcggcgataccgtcgttacctttaccgcccagaccgatcggccattttacggtagagccagtaccaacgttgtttttccactcttcgttcactttcgccaggtagctggtgaagacgaaggaagtcccggagccatctgcgcggcgtactacagcaatgttttgtgaaggcagtttcagacccggattcagtttggcgatggcttcatcatcccacttcttgattttgcccaggtagatgtcgccgagggttttaccatccagcaccagttcgccagacttcagccctggaatgttaaccgccagcaccacgccgccaatcacggtcgggaactggaacagaccttcctgagccagtttttcgtcagacagcggcgcgtcagaggcaccaaaatcaacggtattagcgataatctgttttacgccaccggaagaaccgataccctggtagttaactttattaccggtttctttctggtaagtgtcagcccatttggcatacaccggcgcagggaaggttgcacctgcacctgtcaggcttgcttctgcaaacacagagaaagcactcatcgataaggtcgcggcgacaacagttgcgacggtggtacgcataactttcataatgtctcctgggaggattcataaagcattgtttgttggctacgagaagcaaaataggacaaacaggtgacagttatatgtaaggaatatgacagttttatgacagagagataaagtcttcagtctgatttaaataagcgttgatattcagtcaattacaaacattaataacgaagagatgacagaaaaattttcattctgtgacagagaaaaagtagccgaagatgacggtttgtcacatggagttggcaggatgtttgattaaaatgaagcctgcttttttatactaagttggcattataaaaaagcattgcttatcaatttgttgcaacgaacaggtcactatcagtcaaaataaaatcattatttgatttcgaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctccggataagtagacagcctgataagtcgcacgaaaaacaggtattgacaacatgaagtaacatgcagtaagatacaaatcgctaggtaacactagcagcgtcaaccgggcgctctagctagagccaagctagcttggccggatccgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgtcgaagcttaacacagaaaaaagcccgcacctgacagtgcgggctttttttttcgaccactgcagtctgttacaggtcactaataccatctaagtagttgattcatagtgactgcatatgttgtgttttacagtattatgtagtctgttttttatgcaaaatctaatttaatatattgatatttatatcattttacgtttctcgttcagcttttttatactaacttgagcggcccttgacgatgccacatcctgagcaaataattcaaccactaattgtgagcggataacacaaggaggaaacagctatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttccttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtctacaaataaaaaaggcacgtcagatgacgtgccttttttcttggggccgaaaaatgccccgcttacgcagggcatccatttattactcaaccgtaaccgattttgccaggttacgcggctggtcaacgtcggtgcctttgatcagcgcgacatggtaagccagcagctgcagcggaacggtgtagaagatcggtgcaatcacctcttccacatgcggcatctcgatgatgtgcatgttatcgctacttacaaaacccgcatcctgatcggcgaagacatacaactgaccgccacgcgcgcgaacttcttcaatgttggatttcagtttttccagcaattcgttgttcggtgcaacaacaataaccggcatatcggcatcaattagcgccagcggaccgtgtttcagttcgccagcagcgtaggcttcagcgtgaatgtaagagatctctttcaacttcaatgcgccttccagcgcgattgggtactgatcgccacggcccaggaacagcgcgtgatgtttgtcagagaaatcttctgccagcgcttcaatgcgtttgtcctgagacagcatctgctcaatacggctcggcagcgcctgcagaccatgcacgatgtcatgttcaatggaggcatccagacctttcaggcgagacagcttcgccaccagcatcaacagcacagttaactgagtggtgaatgctttagtggatgccacgccgatttctgtacccgcgttggtcattagcgccagatcggattcgcgcaccagagaagaacccggaacgttacagattgccagtgaaccaaggtaacccagctctttcgacagacgcaggccagccagggtatccgcggtttcgccagactgtgacaaggtgatcatcaggctgttacgacgcacggcagatttgcgatagcggaattcagaggcgatttcgacgtcgcacggaatacctgctagcgattcaaaccagtagcgggaaaccataccggagttataagaagtaccacaggcgaggatctgaatatgctcaaccttcgacagcagttcgtcggcgttcggtcccagctcgcttaaatcaacctgaccgtggctgatgcgtccggtaagggtgtttttgatcgcgttcggctgttcgtagatctctttctgcatgtagtgacggtaaatgcctttatcgcccgcgtcatattgcagattggattcgatatcctgacgttttacttccgcgccagttttatcgaagatgtttaccgaacggcgagtga

SEQ ID NO: 58 - DNA, 40 bp, Artificial Sequence
  Primer (Tn7dS_6038-7-1)
tcgagctcggtaccctgtttttccactcttcgttcacttt

SEQ ID NO: 59 - DNA, 33 bp, Artificial Sequence
  Primer (Tn7dA_6038-7-2)
aggcttcattttaatcaaacatcctgccaactc

SEQ ID NO: 60 - DNA, 25 bp, Artificial Sequence
  Primer (Tn7dattLcmS_6038-7-4)
attaaaatgaagcctgcttttttat

SEQ ID NO: 61 - DNA, 32 bp, Artificial Sequence
  Primer (PgiattRcmA_6038-7-5)
ggcatcgtcaagggccgctcaagttagtataa

SEQ ID NO: 62 - DNA, 99 bp, Artificial Sequence
  Primer (gi1.2-MVK-S_6038-7-6
gcccttgacgatgccacatcctgagcaaataattcaaccactaattgtgagcggataacacaaggaggaaacagctatgtcattaccgttcttaacttc

SEQ ID NO: 63 - DNA, 96 bp, Artificial Sequence
  Primer (pMW-TaspA-yIDIA_6038-7-7)
ctctagaggatccccggccccaagaaaaaaggcacgtcatctgacgtgccttttttatttgtagacgcgttgttatagcattctatgaatttgcct

SEQ ID NO: 64 - DNA, 36 bp, Artificial Sequence
  Primer (Tn7upSv02_6038-24-1)
atcctctagagtcgaaagaaaaatgccccgcttacg

SEQ ID NO: 65 - DNA, 37 bp, Artificial Sequence
  Primer (Tn7upAv02_6038-24-2)
atgcctgcaggtcgactgtcacagtctggcgaaaccg

SEQ ID NO: 66 - DNA, 20 bp, Artificial Sequence
  Primer (Tn7v02-F_6038-22-5)
acgaactgctgtcgaaggtt

SEQ ID NO: 67 - DNA, 20 bp, Artificial Sequence
  Primer (Tn7v02-R_6038-22-6)
ggtgtacgccaggttgttct

SEQ ID NO: 68 - DNA, 2414 bp, Artificial Sequence; identical to SEQ ID NO: 1
  Nucleotide sequence of genomic fragment containing attL-Tet-attR-Ptac

SEQ ID NO: 69 - DNA, 7464 bp, Artificial Sequence
  Nucleotide sequence containing genomic sequence encoding downstream enzymes in the mevalonate pathway under control of tac promoter
ttagtacagcggcttaccgctactgtctttaatattggtcttccacgcagcgcgaacctgttcaactacactatccggcaggctggcgtaatccaggtcgttcgcctgtttagccccggttttgtacgcccagtcgaagaatttcagcacttctgtgccttgttctggtttcttctgatctttgtggatcagaatgaacgtggtagaggtaataggccatgcatcttcgcctttctggttggtcagatcctgagcgaaggttttgctccagtctgcaccttttgctgcattagcgaagttttcttcggtcggactaaccggtttaccatcagcggagatcagtttggtgtacgccaggttgttctgcttcgcgtaagcatattcaacataaccaattgcacccggcagacgctgaacgaacgcggcgataccgtcgttacctttaccgcccagaccgatcggccattttacggtagagccagtaccaacgttgtttttccactcttcgttcactttcgccaggtagctggtgaagacgaaggaagtcccggagccatctgcgcggcgtactacagcaatgttttgtgaaggcagtttcagacccggattcagtttggcgatggcttcatcatcccacttcttgattttgcccaggtagatgtcgccgagggttttaccatccagcaccagttcgccagacttcagccctggaatgttaaccgccagcaccacgccgccaatcacggtcgggaactggaacagaccttcctgagccagtttttcgtcagacagcggcgcgtcagaggcaccaaaatcaacggtattagcgataatctgttttacgccaccggaagaaccgataccctggtagttaactttattaccggtttctttctggtaagtgtcagcccatttggcatacaccggcgcagggaaggttgcacctgcacctgtcaggcttgcttctgcaaacacagagaaagcactcatcgataaggtcgcggcgacaacagttgcgacggtggtacgcataactttcataatgtctcctgggaggattcataaagcattgtttgttggctacgagaagcaaaataggacaaacaggtgacagttatatgtaaggaatatgacagttttatgacagagagataaagtcttcagtctgatttaaataagcgttgatattcagtcaattactgaagcctgcttttttatactaacttgagcgagatctccctgttgacaattaatcatcggctctataatgtgtggaatcgtgagcggataacaatttcacacaaggagactgccatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttccttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtctacaaataaaaaaggcacgtcagatgacgtgccttttttcttggggccgaaaaatgccccgcttacgcagggcatccatttattactcaaccgtaaccgattttgccaggttacgcggctggtcaacgtcggtgcctttgatcagcgcgacatggtaagccagcagctgcagcggaacggtgtagaagatcggtgcaatcacctcttccacatgcggcatctcgatgatgtgcatgttatcgctacttacaaaacccgcatcctgatcggcgaagacatacaactgaccgccacgcgcgcgaacttcttcaatgttggatttcagtttttccagcaattcgttgttcggtgcaacaacaataaccggcatatcggcatcaattagcgccagcggaccgtgtttcagttcgccagcagcgtaggcttcagcgtgaatgtaagagatctctttcaacttcaatgcgccttccagcgcgattgggtactgatcgccacggcccaggaacagcgcgtgatgtttgtcagagaaatcttctgccagcgcttcaatgcgtttgtcctgagacagcatctgctcaatacggctcggcagcgcctgcagaccatgcacgatgtcatgttcaatggaggcatccagacctttcaggcgagacagcttcgccaccagcatcaacagcacagttaactgagtggtgaatgctttagtggatgccacgccgatttctgtacccgcgttggtcattagcgccagatcggattcgcgcaccagagaagaacccggaacgttacagattgccagtgaaccaaggtaacccagctctttcgacagacgcaggccagccagggtatccgcggtttcgccagactgtgacaaggtgatcatcaggctgttacgacgcacggcagatttgcgatagcggaattcagaggcgatttcgacgtcgcacggaatacctgctagcgattcaaaccagtagcgggaaaccataccggagttataagaagtaccacaggcgaggatctgaatatgctcaaccttcgacagcagttcgtcggcgttcggtcccagctcgcttaaatcaacctgaccgtggctgatgcgtccggtaagggtgtttttgatcgcgttcggctgttcgtagatctctttctgcatgtagtgacggtaaatgcctttatcgcccgcgtcatattgcagattggattcgatatcctgacgttttacttccgcgccagttttatcgaagatgtttaccgaacggcgagtga

SEQ ID NO: 70 - DNA, 70 bp, Artificial Sequence
  Primer (APtacKKDyIv03_6038-36-5)
gataaagtcttcagtctgatttaaataagcgttgatattcagtcaattactgaagcctgcttttttatac

SEQ ID NO: 71 - DNA, 71 bp, Artificial Sequence
  Primer (SPtacKKDyIv02_6038-36-3)
tcaccaaaaataataacctttcccggtgcagaagttaagaacggtaatgacatggcagtctccttgtgtga

SEQ ID NO: 72 - protein, 803 aa, Enterococcus faecalis
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 73 - DNA, 2412 bp, Enterococcus faecalis
atgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataa

SEQ ID NO: 74 - DNA, 2412 bp, Artificial Sequence
  DNA having modified codons, which encodes mvaE derived from Enterococcus faecalis
atgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaa

SEQ ID NO: 75 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 76 - DNA, 1152 bp, Enterococcus faecalis
atgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaa

SEQ ID NO: 77 - DNA, 1152 bp, Artificial Sequence
  DNA having modified codons, which encodes mvaS derived from Enterococcus faecalis
atgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaa

SEQ ID NO: 78 - DNA, 40 bp, Artificial Sequence
  Primer for amplifying a fragment comprising Para composed of araC and ara BAD promoters from E.coli
tgaattcgagctcggtacccactcttcctttttcaatatt

SEQ ID NO: 79 - DNA, 40 bp, Artificial Sequence
  Primer for amplifying a fragment comprising Para composed of araC and ara BAD promoters from E.coli
ataataaccacggttttcattttttataacctccttagag

SEQ ID NO: 80 - DNA, 40 bp, Artificial Sequence
  Primer for amplifying a fragment comprising EFmvaE gene
ctctaaggaggttataaaaaatgaaaaccgtggttattat

SEQ ID NO: 81 - DNA, 55 bp, Artificial Sequence
  Primer for amplifying a fragment comprising EFmvaE gene
ttatcgataccaatggtcatgtttttttacctcctttactgtttgcgcagatcat

SEQ ID NO: 82 - DNA, 55 bp, Artificial Sequence
  Primer for amplifying a fragment comprising EFmvaS gene
atgatctgcgcaaacagtaaaggaggtaaaaaaacatgaccattggtatcgataa

SEQ ID NO: 83 - DNA, 40 bp, Artificial Sequence
  Primer for amplifying a fragment comprising EFmvaS gene
cagcggaactggcggctcccttaattgcggtaactacgca

SEQ ID NO: 84 - DNA, 40 bp, Artificial Sequence
  Primer for amplifying a fragment comprising Ttrp
tgcgtagttaccgcaattaagggagccgccagttccgctg

SEQ ID NO: 85 - DNA, 38 bp, Artificial Sequence
  Primer for amplifying a fragment comprising Ttrp
gtcgactctagaggatccctaatgagaattagtcaaat

SEQ ID NO: 86 - DNA, 108 bp, Artificial Sequence
  Primer 1 for pUC-mvk-pmk (KKDS1_6038-3-1)
gaggaataaaccatggatccgagctcggatccactagtaacggccgccagtgtgctggaattcgcccttaggaggtaaaaaaacatgtcattaccgttcttaacttct

SEQ ID NO: 87 - DNA, 55 bp, Artificial Sequence; identical to SEQ ID NO: 52
  Primer 2 for pUC-mvk-pmk (KKMyIA_6038-2-9)

SEQ ID NO: 88 - DNA, 51 bp, Artificial Sequence; identical to SEQ ID NO: 53
  Primer 1 for pTWV-dmd-yidi (KMS_6038-6-1)

SEQ ID NO: 89 - DNA, 47 bp, Artificial Sequence; identical to SEQ ID NO: 54
  Primer 2 for pTWV-dmd-yidi (KDyIA_6038-3-3)

SEQ ID NO: 90 - DNA, 9329 bp, Artificial Sequence
  Nucleotide sequence of the expression vector, pTrc-KKDyI(alpha)
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcggatccactagtaacggccgccagtgtgctggaattcgcccttaggaggtaaaaaaacatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataactgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 91 - DNA, 68 bp, Artificial Sequence
  Primer for amplifying IspSK gene (pTrcKKDyIkSS_6038-10-1)
atagaatgctataacaacgcgtcctgcattcgcccttaggaggtaaaaaaacatgtgtgcgacctctt

SEQ ID NO: 92 - DNA, 46 bp, Artificial Sequence
  Primer for amplifying IspSK gene (pTrcKKDyIkSS_6038-10-2)
ccatatggtaccagctgcagttagacatacatcagctggttaatcg

SEQ ID NO: 93 - DNA, 11062 bp, Artificial Sequence
  Nucleotide sequence of the expression vector, pTrc-KKDyI(alpha)-ispS(K)
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcggatccactagtaacggccgccagtgtgctggaattcgcccttaggaggtaaaaaaacatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcctgcacccttaaggaggaaaaaaacatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagctgcatgcagaattcgcccttaaggaggaaaaaaaaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgctgcatcgcccttaggaggtaaaaaaaaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtcctgcattcgcccttaggaggtaaaaaaacatgtgtgcgacctcttctcaatttactcagattaccgagcataattcccgtcgttccgcaaactatcagccaaacctgtggaatttcgaattcctgcaatccctggagaacgacctgaaagtggaaaagctggaggagaaagcgaccaaactggaggaagaagttcgctgcatgatcaaccgtgtagacacccagccgctgtccctgctggagctgatcgacgatgtgcagcgcctgggtctgacctacaaatttgaaaaagacatcattaaagccctggaaaacatcgtactgctggacgaaaacaaaaagaacaaatctgacctgcacgcaaccgctctgtctttccgtctgctgcgtcagcacggtttcgaggtttctcaggatgtttttgagcgtttcaaggataaagaaggtggtttcagcggtgaactgaaaggtgacgtccaaggcctgctgagcctgtatgaagcgtcttacctgggtttcgagggtgagaacctgctggaggaggcgcgtaccttttccatcacccacctgaagaacaacctgaaagaaggcattaataccaaggttgcagaacaagtgagccacgccctggaactgccatatcaccagcgtctgcaccgtctggaggcacgttggttcctggataaatacgaaccgaaagaaccgcatcaccagctgctgctggagctggcgaagctggattttaacatggtacagaccctgcaccagaaagagctgcaagatctgtcccgctggtggaccgagatgggcctggctagcaaactggattttgtacgcgaccgcctgatggaagtttatttctgggcactgggtatggcgccagacccgcagtttggtgaatgtcgcaaagctgttactaaaatgtttggtctggtgacgatcatcgatgacgtgtatgacgtttatggcactctggacgaactgcaactgttcaccgatgctgtagagcgctgggacgttaacgctattaacaccctgccggactatatgaaactgtgtttcctggcactgtacaacaccgttaacgacacgtcctattctattctgaaagagaaaggtcataacaacctgtcctatctgacgaaaagctggcgtgaactgtgcaaagcctttctgcaagaggcgaaatggtccaacaacaaaattatcccggctttctccaagtacctggaaaacgccagcgtttcctcctccggtgtagcgctgctggcgccgtcttacttttccgtatgccagcagcaggaagacatctccgaccacgcgctgcgttccctgaccgacttccatggtctggtgcgttctagctgcgttatcttccgcctgtgcaacgatctggccacctctgcggcggagctggaacgtggcgagactaccaattctatcattagctacatgcacgaaaacgatggtaccagcgaggaacaggcccgcgaagaactgcgtaaactgatcgacgccgaatggaaaaagatgaatcgtgaacgcgttagcgactccaccctgctgcctaaagcgttcatggaaatcgcagttaacatggcacgtgtttcccactgcacctaccagtatggcgatggtctgggtcgcccagactacgcgactgaaaaccgcatcaaactgctgctgattgaccctttcccgattaaccagctgatgtatgtctaactgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 94 - protein, 301 aa, Methanosarcina mazei
MVSCSAPGKIYLFGEHAVVYGETAIACAVELRTRVRAELNDSITIQSQIGRTGLDFEKHPYVSAVIEKMRKSIPINGVFLTVDSDIPVGSGLGSSAAVTIASIGALNELFGFGLSLQEIAKLGHEIEIKVQGAASPTDTYVSTFGGVVTIPERRKLKTPDCGIVIGDTGVFSSTKELVANVRQLRESYPDLIEPLMTSIGKISRIGEQLVLSGDYASIGRLMNVNQGLLDALGVNILELSQLIYSARAAGAFGAKITGAGGGGCMVALTAPEKCNQVAEAVAGAGGKVTITKPTEQGLKVD

SEQ ID NO: 95 - DNA, 906 bp, Methanosarcina mazei
atggtttcatgttctgcgcccgggaaaatctatcttttcggagaacatgcggttgtttacggagaaaccgcaatagcgtgtgcagtagagttaaggacccgggtgcgggcggagttaaatgactccataactatccagtctcagatcggcaggacaggtcttgattttgaaaaacatccctatgtctctgcagtgattgaaaaaatgagaaaatctatccccataaatggtgtttttttaactgttgattccgacattcctgttgggtcagggctcggctcatctgctgctgttacgattgcaagcataggagctctcaacgaacttttcggattcgggctttcgcttcaggaaattgcgaaactggggcatgaaattgagataaaagttcagggtgcagcgagccctactgacacctatgtttctactttcggaggagtcgttaccatccctgaaaggagaaagcttaagactcctgactgtggaattgttataggggacaccggagttttttcttctacaaaagagcttgtggcaaacgtcaggcagctccgcgaaagttaccctgatcttatcgaacctcttatgacttctattggcaaaatctccagaatcggtgagcaacttgtactttccggggactatgcttctattggcaggcttatgaatgtaaatcagggactgcttgatgcacttggagttaatatccttgagctttcacagcttatctattctgcaagggcagcaggagctttcggggcaaaaattactggagcaggaggcggtggttgtatggttgcgctaactgcaccggagaaatgtaatcaggtagcggaagccgttgcaggtgcagggggcaaagtgaccattacaaaacccacggaacaggggttgaaggtcgattga

SEQ ID NO: 96 - DNA, 906 bp, Artificial Sequence
  DNA having modified codons, which encodes mevalonate kinase derived from Methanosarcina mazei (Mmamvk)
atggtatcctgttctgcgccgggtaagatttacctgttcggtgaacacgccgtagtttatggcgaaactgcaattgcgtgtgcggtggaactgcgtacccgtgttcgcgcggaactcaatgactctatcactattcagagccagatcggccgcaccggtctggatttcgaaaagcacccttatgtgtctgcggtaattgagaaaatgcgcaaatctattcctattaacggtgttttcttgaccgtcgattccgacatcccggtgggctccggtctgggtagcagcgcagccgttactatcgcgtctattggtgcgctgaacgagctgttcggctttggcctcagcctgcaagaaatcgctaaactgggccacgaaatcgaaattaaagtacagggtgccgcgtccccaaccgatacgtatgtttctaccttcggcggcgtggttaccatcccggaacgtcgcaaactgaaaactccggactgcggcattgtgattggcgataccggcgttttctcctccaccaaagagttagtagctaacgtacgtcagctgcgcgaaagctacccggatttgatcgaaccgctgatgacctctattggcaaaatctctcgtatcggcgaacaactggttctgtctggcgactacgcatccatcggccgcctgatgaacgtcaaccagggtctcctggacgccctgggcgttaacatcttagaactgagccagctgatctattccgctcgtgcggcaggtgcgtttggcgctaaaatcacgggcgctggcggcggtggctgtatggttgcgctgaccgctccggaaaaatgcaaccaagtggcagaagcggtagcaggcgctggcggtaaagtgactatcactaaaccgaccgagcaaggtctgaaagtagattaa

SEQ ID NO: 97 - DNA, 79 bp, Artificial Sequence; identical to SEQ ID NO: 18
  Primer for amplifying IspSK gene

SEQ ID NO: 98 - DNA, 42 bp, Artificial Sequence; identical to SEQ ID NO: 19
  Primer for amplifying IspSK gene

SEQ ID NO: 99 - DNA, 29 bp, Artificial Sequence
  Primer for amplifying pSTV28-Ptac-Ttrp construct
gtgtgaaattgttatccgctcacaattcc

SEQ ID NO: 100 - DNA, 27 bp, Artificial Sequence
  Primer for amplifying pSTV28-Ptac-Ttrp construct
gaattcactggccgtcgttttacaacg

SEQ ID NO: 101 - DNA, 53 bp, Artificial Sequence
  Primer for amplifying Mmamvk gene
ctgatgtatgtctaactgcataaaggaggtaaaaaaacatggtatcctgttct

SEQ ID NO: 102 - DNA, 50 bp, Artificial Sequence
  Primer for amplifying Mmamvk gene
ttgtaaaacgacggccagtgaattcttaatctactttcagaccttgctcg

SEQ ID NO: 103 - DNA, 20 bp, Artificial Sequence
  Primer for amplifying pSTV28-Ptac-IspSK
gaattcactggccgtcgttt

SEQ ID NO: 104 - DNA, 20 bp, Artificial Sequence
  Primer for amplifying pSTV28-Ptac-IspSK
ttagacatacatcagctggt

SEQ ID NO: 105 - DNA, 40 bp, Artificial Sequence
  Primer 1 for amplifying Mclmvk gene (mcl_mvk_N)
tttcacacaaggagactcccatgacgatggcttccgctcc

SEQ ID NO: 106 - DNA, 40 bp, Artificial Sequence
  Primer 2 for amplifying Mclmvk gene (mcl_mvk_C)
cagcggaactggcggctcccttacgcgacttccaggcgaa

SEQ ID NO: 107 - DNA, 20 bp, Artificial Sequence
  Primer 1 for amplifying pMW219-Ptac-Ttrp (PtTt219f)
gggagccgccagttccgctg

SEQ ID NO: 108 - DNA, 21 bp, Artificial Sequence
  Primer 2 for amplifying pMW219-Ptac-Ttrp (PtTt219r)
gggagtctccttgtgtgaaat

SEQ ID NO: 109 - DNA, 68 bp, Artificial Sequence
  Primer
ctgatgaactgtcacctgaatgagtgctgatgaaaatatagaaaggtcatttttcctgaatatgctca

SEQ ID NO: 110 - DNA, 64 bp, Artificial Sequence
  Primer
attcgccagcataacgatgccgctgttgagctgaggaacacgtttgttgacagctggtccaatg

SEQ ID NO: 111 - DNA, 70 bp, Artificial Sequence
  Primer
atgcgcactccttacgtactggctctactggtttctttgcgaaaggtcatttttcctgaatatgctcaca

SEQ ID NO: 112 - DNA, 64 bp, Artificial Sequence
  Primer
ttaaggaatcgcctggaccatcatcggcgagccgttctgacgtttgttgacagctggtccaatg

SEQ ID NO: 113 - DNA, 68 bp, Artificial Sequence
  Primer
atgttgtggatttggaatgccctgatcgttttcgttaccggaaaggtcatttttcctgaatatgctca

SEQ ID NO: 114 - DNA, 64 bp, Artificial Sequence
  Primer
atgacggtctgcgcaaaaaaacacgttcatctcactcgcgcgtttgttgacagctggtccaatg

SEQ ID NO: 115 - DNA, 21 bp, Artificial Sequence
  Primer
gattcccacttcaccgagccg

SEQ ID NO: 116 - DNA, 21 bp, Artificial Sequence
  Primer
ggcaggtatggtgctctgacg

SEQ ID NO: 117 - DNA, 18 bp, Artificial Sequence
  Primer
gcgaagccctctccgttg

SEQ ID NO: 118 - DNA, 21 bp, Artificial Sequence
  Primer
agccagtcagcctcatcagcg

SEQ ID NO: 119 - DNA, 21 bp, Artificial Sequence
  Primer
ccgtgtggttctgaaagccga

SEQ ID NO: 120 - DNA, 21 bp, Artificial Sequence
  Primer
cgttgccgtaaatgtatccgt

SEQ ID NO: 121 - DNA, 27 bp, Artificial Sequence
  Primer
ggatgtaaaccataacactctgcgaac

SEQ ID NO: 122 - DNA, 25 bp, Artificial Sequence
  Primer
gattggtggttgaattgtccgtaac

SEQ ID NO: 123 - DNA, 22 bp, Artificial Sequence
  Primer
tggaaggattcggatagttgag

SEQ ID NO: 124 - DNA, 20 bp, Artificial Sequence
  Primer
ttactgacctgttgatcggc

SEQ ID NO: 125 - DNA, 20 bp, Artificial Sequence
  Primer
ctgaggatgttttccgcctg

SEQ ID NO: 126 - DNA, 89 bp, Artificial Sequence
  Primer
ctgctgccgcctctcctgcacgacatcagctgaactctcctcctctgtctgacgtcatcctgaagcctgcttttttatactaagttggc

SEQ ID NO: 127 - DNA, 90 bp, Artificial Sequence
  Primer
aacggcggcagaggcggtgataaaaaagagacaagcaaaaagcggttttaccaggttcattatatctccttcttaaagttaaacaaaatt

SEQ ID NO: 128 - DNA, 22 bp, Artificial Sequence
  Primer
tggggcacacgcgtttatgagc

SEQ ID NO: 129 - DNA, 22 bp, Artificial Sequence
  Primer
gtaagcgcccggtccgacctta

SEQ ID NO: 130 - DNA, 20 bp, Artificial Sequence
  Primer
tgctgcgggcattgtttccc

SEQ ID NO: 131 - DNA, 20 bp, Artificial Sequence
  Primer
tcggcattggccgggatctc

SEQ ID NO: 132 - DNA, 89 bp, Artificial Sequence
  Primer
tgttcatcacggccaaatagcgttgaggagcattccgaaacgccaaaaagggggcgtacttgaagcctgcttttttatactaagttggc

SEQ ID NO: 133 - DNA, 90 bp, Artificial Sequence
  Primer
aataattacgtagatatcttctggcaggtctttacctgctggcacctggttcaaactcattatatctccttcttaaagttaaacaaaatt

SEQ ID NO: 134 - DNA, 22 bp, Artificial Sequence
  Primer
catgaaaagaggatgggtgttg

SEQ ID NO: 135 - DNA, 22 bp, Artificial Sequence
  Primer
gttatctaccttaagatcctgt

SEQ ID NO: 136 - protein, 176 aa, Escherichia coli
MSLLNVPAGKDLPEDIYVVIEIPANADPIKYEIDKESGALFVDRFMSTAMFYPCNYGYINHTLSLDGDPVDVLVPTPYPLQPGSVIRCRPVGVLKMTDEAGEDAKLVAVPHSKLSKEYDHIKDVNDLPELLKAQIAHFFEHYKDLEKGKWVKVEGWENAEAAKAEIVASFERAKNK

SEQ ID NO: 137 - protein, 199 aa, Pantoea ananatis
MNLVKPLFACLFFITASAAVQAENILSFPQPDSLPDEFYAVTEIPAGGIIKYETDAKTGFIVADRFQSMPVAYPANYGSLTQSLGGDNDPLDVIFYTRAPLAPGTLIKLRPIGVLKMIDGGEVDDKIVAVPASKVDPTYDAIQTVADLPKLEQQRLEAFFRVYKALPEGRKNVELKGFEDASAAKAEIKQAFEAWKAKQ

SEQ ID NO: 138 - protein, 176 aa, Pantoea ananatis
MSLNQVPAGKDLPEDIYVIIEIPANADPIKYEVDKDSGALFVDRFMSTAMFYPCNYGYINHTLSLDGDPVDVLVPTQYPLEPGSVIRCRPVGVLKMTDESGEDAKLVAVPHTKLTKEYDHIKDVNDLPELLRAQITHFFEHYKDLEKGKWVKVDGWDNAEAAKAEIISSFERAAKK

SEQ ID NO: 139 - DNA, 2661 bp, Artificial Sequence
  EFMvaE gene
ttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaa

SEQ ID NO: 140 - DNA, 1401 bp, Artificial Sequence
  EFMvaS gene
ttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaa

SEQ ID NO: 141 - DNA, 5314 bp, Artificial Sequence
  pUC57-EFmvaE
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 142 - DNA, 4054 bp, Artificial Sequence
  pUC57-EFmvaS
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtaccttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 143 - DNA, 38 bp, Artificial Sequence
  Primer to amplify trc promoter (Ptrc)
gaattcgagctcggtacccttgacaattaatcatccgg

SEQ ID NO: 144 - DNA, 45 bp, Artificial Sequence
  Primer to amplify trc promoter (Ptrc)
aaccacggttttcatgtttttttacctcctgagctcggatccatg

SEQ ID NO: 145 - DNA, 45 bp, Artificial Sequence
  Primer to amplify mvaE
catggatccgagctcaggaggtaaaaaaacatgaaaaccgtggtt

SEQ ID NO: 146 - DNA, 55 bp, Artificial Sequence; identical to SEQ ID NO: 81
  Primer to amplify mvaE, mvaS

SEQ ID NO: 147 - DNA, 55 bp, Artificial Sequence; identical to SEQ ID NO: 82
  Primer to amplify mvaS

SEQ ID NO: 148 - DNA, 40 bp, Artificial Sequence; identical to SEQ ID NO: 83
  Primer to amplify tryptophane terminator (Ttrp)

SEQ ID NO: 149 - DNA, 40 bp, Artificial Sequence; identical to SEQ ID NO: 84
  Primer to amplify tryptophane terminator (Ttrp)

SEQ ID NO: 150 - DNA, 38 bp, Artificial Sequence; identical to SEQ ID NO: 85
  Primer

SEQ ID NO: 151 - DNA, 8053 bp, Artificial Sequence
  pMW-Ptrc-mvaES-Ttrp
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcaggaggtaaaaaaacatgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaaaggaggtaaaaaaacatgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaagggagccgccagttccgctggcggcattttaactttctttaatgaagccggaaaaatcctaaattcatttaatatttatctttttaccgtttcgcttaccccggtcgaacgtcaacttacgtcatttttccgcccaacagtaatataatcaaacaaattaatcccgcaacataacaccagtaaaatcaataattttctctaagtcacttattcctcaggtaattgttaatatatccagaatgttcctcaaaatatattttccctctatcttctcgttgcgcttaatttgactaattctcattagggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcatagctcacgctgtaggtatctcagttcggtgtaggtcgttcgctccaagctgggctgtgtgccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgccaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcactaccgcattaaagcatatcgatgataagctgtcaaacatgagcgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 152 - DNA, 40 bp, Artificial Sequence; identical to SEQ ID NO: 80
  Primer to amplify pMW-Ptrc-mvaES-Ttrp

SEQ ID NO: 153 - DNA, 40 bp, Artificial Sequence
  Primer to amplify pMW-Ptrc-mvaES-Ttrp
aatattgaaaaaggaagagtgggtaccgagctcgaattca

SEQ ID NO: 154 - DNA, 40 bp, Artificial Sequence; identical to SEQ ID NO: 78
  Primer to amplify pKD46

SEQ ID NO: 155 - DNA, 40 bp, Artificial Sequence; identical to SEQ ID NO: 79
  Primer to amplify pKD46

SEQ ID NO: 156 - DNA, 9180 bp, Artificial Sequence
  pMW-Para-mvaES-Ttrp
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccactcttcctttttcaatattattgaagcatttatcagggttattgtctcatgagcggatacatatttgaatgtatttagaaaaataaacaaataggggttccgcgcacatttccccgaaaagtgccacctgcatcgatttattatgacaacttgacggctacatcattcactttttcttcacaaccggcacggaactcgctcgggctggccccggtgcattttttaaatacccgcgagaagtagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaaaggaggtaaaaaaacatgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaagggagccgccagttccgctggcggcattttaactttctttaatgaagccggaaaaatcctaaattcatttaatatttatctttttaccgtttcgcttaccccggtcgaacgtcaacttacgtcatttttccgcccaacagtaatataatcaaacaaattaatcccgcaacataacaccagtaaaatcaataattttctctaagtcacttattcctcaggtaattgttaatatatccagaatgttcctcaaaatatattttccctctatcttctcgttgcgcttaatttgactaattctcattagggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctttctcatagctcacgctgtaggtatctcagttcggtgtaggtcgttcgctccaagctgggctgtgtgccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgccaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcactaccgcattaaagcatatcgatgataagctgtcaaacatgagcgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 157 - DNA, 42 bp, Artificial Sequence
  Primer (Linker-F)
agctttagggataacagggtaatctcgagctgcaggcatgca

SEQ ID NO: 158 - DNA, 42 bp, Artificial Sequence
  Primer (Linker-R)
agcttgcatgcctgcagctcgagattaccctgttatccctaa

SEQ ID NO: 159 - DNA, 54 bp, Artificial Sequence
  Primer (phoC5fCAS)
tttttaagctttagggataacagggtaatctcgagtggataacctcatgtaaac

SEQ ID NO: 160 - DNA, 44 bp, Artificial Sequence
  Primer (phoC3fCAS)
tttttaagcttgcatgcctgcagttgatgtctgattatctctga

SEQ ID NO: 161 - DNA, 33 bp, Artificial Sequence
  Primer (n67)
tgcgaagacgtcctcgtgaagaaggtgttgctg

SEQ ID NO: 162 - DNA, 36 bp, Artificial Sequence
  Primer (n68)
tgcgaagggccccgttgtgtctcaaaatctctgatg

SEQ ID NO: 163 - DNA, 70 bp, Artificial Sequence; identical to SEQ ID NO: 111
  Primer (ampH-attL-phi80)

SEQ ID NO: 164 - DNA, 64 bp, Artificial Sequence; identical to SEQ ID NO: 112
  Primer (ampH-attR-phi80)

SEQ ID NO: 165 - DNA, 68 bp, Artificial Sequence; identical to SEQ ID NO: 109
  Primer (DampC-phL)

SEQ ID NO: 166 - DNA, 64 bp, Artificial Sequence; identical to SEQ ID NO: 110
  Primer (DampC-phR)

SEQ ID NO: 167 - DNA, 18 bp, Artificial Sequence
  Primer (ampH-t1)
gcgaagccctctccgttg

SEQ ID NO: 168 - DNA, 21 bp, Artificial Sequence
  Primer (ampH-t2)
agccagtcagcctcatcagcg

SEQ ID NO: 169 - DNA, 21 bp, Artificial Sequence
  Primer (ampC-t1)
gattcccacttcaccgagccg

SEQ ID NO: 170 - DNA, 21 bp, Artificial Sequence
  Primer (ampC-t2)
ggcaggtatggtgctctgacg

SEQ ID NO: 171 - DNA, 64 bp, Artificial Sequence; identical to SEQ ID NO: 114
  Primer (crtE-attRphi80)

SEQ ID NO: 172 - DNA, 68 bp, Artificial Sequence; identical to SEQ ID NO: 113
  Primer (crtZ-attLphi80)

SEQ ID NO: 173 - DNA, 21 bp, Artificial Sequence
  Primer (crtZ-test)
ccgtgtggttctgaaagccga

SEQ ID NO: 174 - DNA, 21 bp, Artificial Sequence
  Primer (crtE-test)
cgttgccgtaaatgtatccgt

SEQ ID NO: 175 - DNA, 40 bp, Artificial Sequence
  Primer (pTac-MGppa-F)
cacacaaggagactcccatgagcttactcaacgtccctgc

SEQ ID NO: 176 - DNA, 40 bp, Artificial Sequence
  Primer (Ttrp-MGppa-R)
cggaactggcggctcccttatttattctttgcgcgctcga

SEQ ID NO: 177 - DNA, 45 bp, Artificial Sequence
  Primer (Tac-CRIM-km-NF3)
cgactctagaggatcagatctccctgttgacaattaatcatcggc

SEQ ID NO: 178 - DNA, 45 bp, Artificial Sequence
  Primer (Ttrp_CRIM_Km-CR2)
tcaaacatgagaattccggcttcattaaagaaagttaaaatgccg

SEQ ID NO: 179 - DNA, 80 bp, Artificial Sequence
  Primer (ydcI_phi80-F)
atggaaaaaaatatccttttcaatcagcgcattcgcttgcgtcatttacagaaaggtcatttttcctgaatatgctcaca

SEQ ID NO: 180 - DNA, 80 bp, Artificial Sequence
  Primer (ydcI_phi80-R)
ctgtgaaatttacgaatagtactcatcagaacctcagcgctcaggcttaatcgtttgttgacagctggtccaatgccagc

SEQ ID NO: 181 - DNA, 22 bp, Artificial Sequence
  Primer (ydcI_F)
cccgataaaaagtacacattgt

SEQ ID NO: 182 - DNA, 22 bp, Artificial Sequence
  Primer (ydcI_R)
agttatctggatcgttggctac

SEQ ID NO: 183 - DNA, 22 bp, Artificial Sequence
  Primer (MG-ppa-seq-1)
gccggtgaagatgcgaaactgg

SEQ ID NO: 184 - DNA, 3471 bp, Artificial Sequence
  Sequence of the chemically synthesized DNA fragment retaining artificial KDyI operon with optimized codons
gcatgcaggaggtatgaatgtcagagttgcgtgccttcagtgccccagggaaagcgttactcgctggtggatatttagttttagatacaaaatatgaagcatttgtagtcggattatcggcacgtatgcacgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatcgtaacttgttcgttattgatattttctctgatgatgcctaccattctcaggaggatagcgttaccgaacatcgtggcaaccgccgtttgagttttcattcgcaccgtattgaagaagttcccaaaacagggctgggctcctcggcaggtttagtcacagttttaactacagctttggcctccttttttgtatcggacctggaaaataatgtagacaaatatcgtgaagttattcataatttagcacaagttgctcattgtcaagctcagggtaaaattggaagcgggtttgatgtagcggcggcagcatatggatctatccgttatcgccgtttcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcctcaaaatatatacagaactcgatcatgcaaattctcgttttatggatggactctctaaactcgatcgcttacacgagactcatgacgattacagcgatcagatatttgagtctcttgagcgtaatgactgtacctgtcaaaagtatcctgaaatcacagaagttcgtgatgcagttgccacaattcgtcgttcctttcgtaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttcgtgctcaaaccgctaatgacaaacgtttttctaaggttcaatggctggatgtaactcaggctgactggggtgttcgtaaagaaaaagatccggaaacttatcttgataaataactgcagaggaggtatgaatgaccgtttacacagcatccgttaccgcacccgtcaacatcgcaacccttaagtattgggggaaacgtgacacgaagttgaatctgcccaccaattcgtccatatcagtgactttatcgcaagatgacctccgtacgttgacctctgcggctactgcacctgagtttgaacgcgacactttgtggttaaatggagaaccacacagcatcgacaatgaacgtactcaaaattgtctgcgcgacctccgccaattacgtaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctcgtatagcacgtaaggggtctggttcagcttgtcgttcgttgtttggcggatacgtggcctgggaaatgggaaaagctgaagatggtcatgattccatggcagtacaaatcgcagacagctctgactggcctcagatgaaagcatgtgtccttgttgtcagcgatattaaaaaggatgtgagttccactcagggtatgcaattgaccgtggcaacctccgaactctttaaagaacgtattgaacatgtcgtaccaaagcgttttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgcccgtgtgattttaactcaagtcggttcaggcccacaagaaacaaacgaatctttgattgacgcaaagactggtctcccaaaggaataaggatccaggaggtatgaatgactgccgacaacaatagtatgccccatggtgcagtatctagttacgccaaattagtgcaaaaccaaacacctgaagacattttggaagagtttcctgaaattattccattacaacaacgtcctaatacccgctctagtgagacgtcaaatgacgaaagcggagaaacatgtttttctggtcatgatgaggagcaaattaagttaatgaatgaaaattgtattgttttggattgggacgataatgctattggtgccggcaccaagaaagtttgtcatttaatggaaaatattgaaaagggtttacttcatcgtgcattctccgtctttattttcaatgaacaaggtgaattacttttacaacaacgtgccactgaaaaaataactttccctgatctttggactaacacatgctgctctcatccactttgtattgatgacgaattaggtttgaagggtaagctcgacgataagattaagggcgctattactgcggcggtgcgtaaactcgatcatgaattaggtattccagaagatgaaactaagacacgtggtaagtttcactttttaaaccgtatccattacatggcaccaagcaatgaaccatggggtgaacatgaaattgattacatcctcttttataagatcaacgctaaagaaaacttgactgtcaacccaaacgtcaatgaagttcgtgacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgaccgtcaaattcatcgtatgctctaaggtacc